(19) United States
(12) Patent Application Publication
(10) Pub. No.: US 2010/0093692 A1
(43) Pub. Date: Apr. 15, 2010

(54) PIPERIDINYL-PIPERIDINE AND PIPERAZINYL-PIPERIDINE FOR USE IN THE TREATMENT OF DIABETES OR PAIN

(75) Inventors: Robert G. Aslanian, Rockaway, NJ (US); Jean E. Lachowicz, Berkeley Heights, NJ (US); Michael Y. Berlin, Flemington, NJ (US); Joyce J. Hwa, Westfield, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(73) Assignee: Schering Corporation Sechering-Plough Corporation, Kenilworth, NJ (US)

(21) Appl. No.: 12/527,499

(22) PCT Filed: Feb. 27, 2008
(86) PCT No.: PCT/US08/02590

§ 371 Date: Oct. 30, 2009

Related U.S. Application Data
(60) Provisional application No. 60/904,718, filed on Mar. 2, 2007.

(51) Int. Cl. A61K 31/4545; A61K 31/496; A61K 31/5377; A61K 31/397; A61P 3/10

(52) U.S. Cl. 514/210.18; 514/316; 514/253.13; 514/237.2

(57) ABSTRACT

The present invention relates to Compounds of Formula (I), compositions comprising the compounds, and methods of using the compounds to treat or prevent pain, diabetes, a diabetic complication, impaired glucose tolerance (IGT) or impaired fasting glucose (IFG) in a patient.

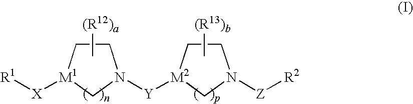

### FIELD OF THE INVENTION

The present invention relates to piperidine derivatives, compositions comprising the piperidine derviatives, and methods of using the piperidine derivatives to treat or prevent pain, diabetes, a diabetic complication, impaired glucose tolerance (IGT) or impaired fasting glucose (IFG) in a patient.

### BACKGROUND OF THE INVENTION

Diabetes refers to a disease process derived from multiple causative factors and is characterized by elevated levels of plasma glucose, or hyperglycemia in the fasting state or after administration of glucose during an oral glucose tolerance test. Persistent or uncontrolled hyperglycemia is associated with increased and premature morbidity and mortality. Abnormal glucose homeostasis is associated with alterations of lipid, lipoprotein and apolipoprotein metabolism and other metabolic and hemodynamic disease. As such, the diabetic patient is at increased risk of macrovascular and microvascular complications, including coronary heart disease, stroke, peripheral vascular disease, hypertension, nephropathy, neuropathy, and retinopathy. Accordingly, therapeutic control of glucose homeostasis, lipid metabolism and hypertension are critically important in the clinical management and treatment of diabetes mellitus.

There are two generally recognized forms of diabetes. In type 1 diabetes, or insulin-dependent diabetes mellitus (IDDM), patients produce little or no insulin, the hormone which regulates glucose utilization. In type 2 diabetes, or noninsulin dependent diabetes mellitus (NIDDM), patients often have plasma insulin levels that are the same or even elevated compared to nondiabetic subjects; however, these patients have developed a resistance to the insulin stimulating effect on glucose and lipid metabolism in the main insulin-sensitive tissue (muscle, liver and adipose tissue), and the plasma insulin levels, while elevated, are insufficient to overcome the pronounced insulin resistance.

Insulin resistance is not associated with a diminished number of insulin receptors but rather to a post-insulin receptor binding defect that is not well understood. This resistance to insulin responsiveness results in insufficient insulin activation of glucose uptake, oxidation and storage in muscle, and inadequate insulin repression of lipolysis in adipose tissue and of glucose production and secretion in the liver.

The available treatments for type 2 diabetes, which have not changed substantially in many years, have recognized limitations. While physical exercise and reductions in dietary intake of calories can dramatically improve the diabetic condition, compliance with this treatment is very poor because of well-entrenched sedentary lifestyles and excess food consumption, especially of foods containing high amounts of saturated fat. Increasing the plasma level of insulin by administration of sulfonylureas (e.g. tolbutamide and glipizide) or meglitinide, which stimulate the pancreatic [beta]-cells to secrete more insulin, and/or by injection of insulin when sulfonylureas or meglitinide become ineffective, can result in insulin concentrations high enough to stimulate the very insulin-resistant tissues. However, dangerously low levels of plasma glucose can result from administration of insulin or insulin secretagogues (sulfonylureas or meglitinide), and an increased level of insulin resistance due to the even higher plasma insulin levels can occur. The biguanides are a separate class of agents that can increase insulin sensitivity and bring about some degree of correction of hyperglycemia. These agents, however, can induce lactic acidosis, nausea and diarrhea.

The glitazones (i.e. 5-benzylthiazolidine-2,4-diones) are another class of compounds that have proven useful for the treatment of type 2 diabetes. These agents increase insulin sensitivity in muscle, liver and adipose tissue in several animal models of type 2 diabetes, resulting in partial or complete correction of the elevated plasma levels of glucose without occurrence of hypoglycemia. The glitazones that are currently marketed are agonists of the peroxisome proliferator activated receptor (PPAR), primarily the PPAR-gamma subtype. PPAR-gamma agonism is generally believed to be responsible for the improved insulin sensititization that is observed with the glitazones. Newer PPAR agonists that are being tested for treatment of Type II diabetes are agonists of the alpha, gamma or delta subtype, or a combination thereof, and in many cases are chemically different from the glitazones (i.e., they are not thiazolidinediones). Serious side effects (e.g. liver toxicity) have been noted in some patients treated with glitazone drugs, such as troglitazone.

Additional methods of treating the disease are currently under investigation. New biochemical approaches include treatment with alpha-glucosidase inhibitors (e.g. acarbose) and protein tyrosine phosphatase-1B (PTP-1B) inhibitors.

Compounds that are inhibitors of the dipeptidyl peptidase-IV (DPP-IV) enzyme are also under investigation as drugs that may be useful in the treatment of diabetes, and particularly type 2 diabetes.

Despite a widening body of knowledge concerning the treatment of diabetes, there remains a need in the art for small-molecule drugs with increased safety profiles and/or improved efficacy that are useful for the treatment of diabetes and related metabolic diseases. This invention addresses that need.

### SUMMARY OF THE INVENTION

In one aspect, the present invention provides Compounds of Formula (I):

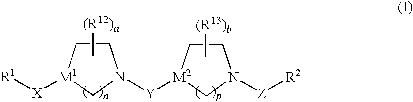

or a pharmaceutically acceptable salt, solvate, ester or prodrug thereof, wherein:

R¹ is aryl, heteroaryl, heterocycloalkyl, alkyl, cycloalkyl or alkylaryl, each of which can be optionally substituted with from 1 to 4 substituents, which are the same or different, and are independently selected from halo, —OH, —O-alkyl, haloalkyl, —OCF₃, —NR⁴R⁵, phenyl, —NO₂, —CO₂R⁴, —CON(R⁴)₂, —S(O)ₘN(R²⁰)₂ and —CN, or R¹ and X are taken together to form:

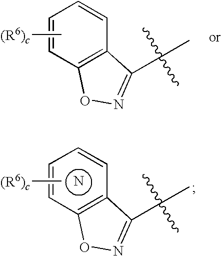

X is —C(O)—, —C(NOR³)—, —C(NNR⁴R⁵)—,

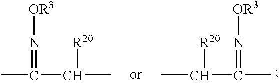

R² is a five or six-membered heteroaryl group, wherein a six-membered heteroaryl group contains 1 or 2 nitrogen ring atoms with the remaining ring atoms being carbon, and a five-membered heteroaryl group contains 1 or 2 hetero ring atoms selected from nitrogen, oxygen, and sulfur, with the remaining ring atoms being carbon; and wherein a five or six membered heteroaryl group can be optionally substituted with from 1 to 3 substituents, which are the same or different, and are independently selected from halo, —OH, alkyl, —O-alkyl, haloalkyl, —OCF₃, —NR⁴R⁵, phenyl, —NO₂, —CO₂R⁴, —CON(R⁴)₂, —CH₂NR⁴R⁵, —(N)C(NR⁴R⁵)₂, and —CN;

R³ is hydrogen, alkyl, aryl, heteroaryl, heterocycloalkyl, arylalkyl, haloalkyl, —CH₂CF_{3,} —(CH₂)ₑ—C(O)N(R⁴)₂, —(CH₂)ₑ—C(O)OR⁴ or —(CH₂)ₑ—C(O)R³⁰, wherein an aryl, heteroaryl or heterocycloalkyl group, or the aryl portion of an arylalkyl group can be optionally substituted with from 1 to 3 substituents, which are the same or different, and are independently selected from halo, —OH, —OCF₃, haloalkyl, —CN, —N(R⁴⁵)₂, —CO₂R⁴⁵ and —C(O)N(R⁴⁵)₂;

each occurrence of R⁴ is independently hydrogen, alkyl, aryl or alkylaryl, wherein an aryl group or the aryl moiety of an alkylaryl group can be optionally substituted with 1 to 3 substituents, which are the same or different, and are independently selected from halo, haloalkyl, —OCF₃, —OH, —N(R⁴⁵)₂, —CO₂R⁴⁵, —C(O)N(R⁴⁵)₂ and —CN;

R⁵ is hydrogen, alkyl, —C(O)R⁴, —C(O)₂R⁴ or —C(O)N(R⁴)₂, or R⁴ and R⁵ taken together with the nitrogen atom to which they are both attached, join to form a five- or six-membered heterocycloalkyl group;

R⁶ is alkyl, aryl, alkylaryl, halo, —OH, —O—(C₁-C₆ alkyl), haloalkyl, —OCF₃, —NR⁴R⁵, phenyl, —NO₂, —CO₂R⁴, —CON(R⁴)₂ or —CN;

R¹² is alkyl, —OH, —O-alkyl, or —F;

R¹³ is alkyl, —OH, —O-alkyl, or —F;

each occurrence of R²⁰ is independently —H or C₁-C₆ alkyl;

R³⁰ is heterocycloalkyl;

each occurrence of R⁴⁵ is independently H, alkyl, alkylaryl, or aryl, wherein an aryl group or the aryl moiety of an alkylaryl group can be optionally substituted with from 1 to 3 substituents which are the same or different, and are independently selected from haloalkyl, —OH, halo, alkyl, —NO₂, and —CN;

M¹ and M² are each independently CH, CF or N;

Y is —CH₂—, —C(O)—, —C(NOR²⁰)— or —C(S)—;

Z is alkylene;

a is 0, 1 or 2;

b is 0, 1 or 2;

c is 0, 1 or 2;

e is an integer ranging from 0 to 5;

m is 1 or 2;

n is 1, 2 or 3, such that when M¹ is nitrogen, n is 2 or 3; and

p is 1, 2 or 3, such that when M² is nitrogen, p is 2 or 3.

In another another aspect, the invention provides a method of treating pain, diabetes, a diabetic complication, impaired glucose tolerance or impaired fasting glucose (each being a “Condition”) in a patient, comprising administering to the patient an effective amount of one or more Compounds of Formula (I).

In a further aspect, the invention provides compositions comprising one or more Compounds of Formula (I), an additional therapeutic agent, and a pharmaceutically acceptable carrier, wherein the amounts of the one or more Compounds of Formula (I) and the additional therapeutic agent are together effective to treat a Condition in a patient.

### BRIEF DESCRIPTION OF THE FIGURES

FIG. 1 shows the effect of Compound 446 and rosiglitazone on non-fasting glucose levels in STZ-induced type 2 diabetic mice. The solid line denoted (▪) represents control mice, the dashed line denoted (▾) represents mice treated with Compound 446 at 10 mg/kg/day, and the solid line denoted (▴) represents mice treated with rosiglitazone at 5 mg/kg/day. The x-axis indicates time (weeks) and the y-axis indicates non-fasting glucose levels (mg/dl).

FIG. 2 shows the effect of Compound 446 and rosiglitazone on HbA1C levels in STZ-induced type 2 diabetic mice. The solid line denoted (▪) represents control mice, the the dashed line denoted (▾) represents mice treated with Compound 446 at 10 mg/kg/day, and the solid line denoted (▴) represents mice treated with rosiglitazone at 5 mg/kg/day. The x-axis indicates time (weeks) and the y-axis indicates HbA1C levels as % glycosylated protein.

FIG. 3 shows the effect of Compound 446 on plasma glucose levels in a rat model of diabetes. The leftmost bar represents untreated control rats and the rightmost bar represents rats treated with Compound 446 (10 mg/kg/day in diet, one week of treatment). The y-axis represents the percent change in glucose levels of the test animals (mg/dl) due to treatment.

FIG. 4 shows the effect of Compound 287 on plasma HbA1c levels in a rat model of diabetes. The leftmost bar represents untreated control rats, the middle gray bar represents rats treated with Compound 287 (68 mg/kg/day in diet, two weeks of treatment), and the rightmost black bar represents rats treated with Compound 287 (68 mg/kg/day in diet, two weeks of treatment). The y-axis represents the percent change in HbA1c levels of the test animals (mg/dl) due to treatment.

### DETAILED DESCRIPTION OF THE INVENTION

As used above, and throughout this disclosure, the following terms, unless otherwise indicated, shall be understood to have the following meanings:

A “patient” is a human or non-human mammal. In one embodiment, a patient is a human. In another embodiment, a patient is a non-human mammal, including, but not limited to, a monkey, dog, baboon, rhesus, mouse, rat, horse, cat or rabbit. In another embodiment, a patient is a companion animal, including but not limited to a dog, cat, rabbit, horse or ferret. In one embodiment, a patient is a dog. In another embodiment, a patient is a cat.

The term “obesity” as used herein, refers to a patient being overweight and having a body mass index (BMI) of 25 or greater. In one embodiment, an obese patient has a BMI of 25 or greater. In another embodiment, an obese patient has a BMI from 25 to 30. In another embodiment, an obese patient has a BMI greater than 30. In still another embodiment, an obese patient has a BMI greater than 40.

The term “impaired glucose tolerance” as used herein, is defined as a two-hour glucose level of 140 to 199 mg per dL (7.8 to 11.0 mmol) as measured using the 75-g oral glucose tolerance test. A patient is said to be under the condition of impaired glucose tolerance when he/she has an intermediately raised glucose level after 2 hours, wherein the level is less than would qualify for type 2 diabetes mellitus.

The term “impaired fasting glucose” as used herein, is defined as a fasting plasma glucose level of 100 to 125 mg/dL; normal fasting glucose values are below 100 mg per dL.

The term “effective amount” as used herein, refers to an amount of Compound of Formula (I) and/or an additional therapeutic agent, or a composition thereof that is effective in producing the desired therapeutic, ameliorative, inhibitory or preventative effect when administered to a patient suffering from a Condition. In the combination therapies of the present invention, an effective amount can refer to each individual agent or to the combination as a whole, wherein the amounts of all agents administered are together effective, but wherein the component agent of the combination may not be present individually in an effective amount.

The term “alkyl,” as used herein, refers to an aliphatic hydrocarbon group which may be straight or branched and which contains from about 1 to about 20 carbon atoms. In one embodiment, an alkyl group contains from about 1 to about 12 carbon atoms. In another embodiment, an alkyl group contains from about 1 to about 6 carbon atoms. Non-limiting examples of alkyl groups include methyl, ethyl, n-propyl, isopropyl, n-butyl, sec-butyl, isobutyl, tert-butyl, n-pentyl, neopentyl, isopentyl, n-hexyl, isohexyl and neohexyl. An alkyl group may be unsubstituted or optionally substituted by one or more substituents which may be the same or different, each substituent being independently selected from the group consisting of halo, alkyl, aryl, cycloalkyl, cyano, —OH, —O-alkyl, -alkylene-O-alkyl, alkylthio, —NH₂, —NH(alkyl), —N(alkyl)₂, —NH(cycloalkyl), —O—C(O)-alkyl, —O—C(O)-aryl, —O—C(O)-cycloalkyl, —C(O)OH and —C(O)O-alkyl. In one embodiment, an alkyl group is unsubstituted. In another embodiment, an alkyl group is linear. In another embodiment, an alkyl group is branched.

The term “alkylene,” as used herein, refers to an alkyl group, as defined above, wherein one of the alkyl group's hydrogen atoms has been replaced with a bond. Non-limiting examples of alkylene groups include —CH₂—, —CH₂CH₂—, —CH₂CH₂CH₂—, —CH₂CH₂CH₂CH₂—, —CH(CH₃)CH₂CH₂— and —CH₂CH(CH₃)CH₂—. In one embodiment, an alkylene group has from 1 to about 6 carbon atoms. In another embodiment, an alkylene group is branched. In another embodiment, an alkylene group is linear.

The term “aryl,” as used herein, refers to an aromatic monocyclic or multicyclic ring system comprising from about 6 to about 14 carbon atoms. In one embodiment, an aryl group contains from about 6 to about 10 carbon atoms. An aryl group can be optionally substituted with one or more “ring system substituents” which may be the same or different, and are as defined herein below. Non-limiting examples of illustrative aryl groups include phenyl and naphthyl. In one embodiment, an aryl group is unsubstituted. In another embodiment, an aryl group is phenyl.

The term “alkylaryl” as used herein, refers to an aryl group, as defined above, joined to an alkyl group, as defined above, wherein an alkylaryl group is bound to the rest of the molecule via it's aryl moiety.

The term “arylalkyl” as used herein, refers to an aryl group, as defined above, joined to an alkyl group, as defined above, wherein an arylalkyl group is bound to the rest of the molecule via it's alkyl moiety. In one embodiment, an arylalkyl group is a benzyl group.

The term “cycloalkyl,” as used herein, refers to a non-aromatic mono- or multicyclic carbocyclic ring system comprising from about 3 to about 10 ring carbon atoms. In one embodiment, a cycloalkyl contains from about 5 to about 10 ring carbon atoms. In another embodiment, a cycloalkyl contains from about 5 to about 7 ring atoms. Non-limiting examples of illustrative monocyclic cycloalkyls include cyclopropyl, cyclobutyl, cyclopentyl, cyclohexyl, cycloheptyl and cyclooctyl. Non-limiting examples of illustrative multicyclic cycloalkyls include 1-decalinyl, norbornyl and adamantyl. A cycloalkyl group can be optionally substituted with one or more “ring system substituents” which may be the same or different, and are as defined herein below. In one embodiment, a cycloalkyl group is unsubstituted.

The term “halo” as used herein, refers to —F, —Cl, —Br or —I.

The term “haloalkyl” as used herein, refers to an alkyl group, as defined above, wherein one or more of the alkyl group's hydrogen atoms have been independently replaced with —F, —Cl, —Br or —I. Non-limiting illustrative examples of haloalkyl groups include —CH₂F, —CHF₂, —CF₃, —CH₂CHF₂, —CH₂CHF₃, —CCl₃, —CHCl₂, —CH₂Cl, and —CH₂CHCl₃.

The term “heteroaryl,” as used herein, refers to an aromatic monocyclic or multicyclic ring system comprising about 5 to about 14 ring atoms, wherein from 1 to 4 of the ring atoms is independently O, N or S and the remaining ring atoms are carbon atoms. In one embodiment, a heteroaryl group has 5 to 10 ring atoms. In another embodiment, a heteroaryl group is monocyclic and has 5 or 6 ring atoms. A heteroaryl group can be optionally substituted by one or more “ring system substituents” which may be the same or different, and are as defined herein below. A heteroaryl group can be joined via a ring carbon atom or a ring nitrogen atom and any ring nitrogen atom of a heteroaryl group can be optionally oxidized to the corresponding N-oxide. The term “heteroaryl” also encompasses a heteroaryl group, as defined above, which has been fused to a benzene ring. Non-limiting examples of illustrative heteroaryl groups include pyridyl (e.g., 2-, 3-, or 4-pyridyl), pyridyl N-oxide (e.g., 2-, 3-, or 4-pyridyl N-oxide), pyrazinyl, furanyl, thienyl, pyrimidinyl, pyridone (including N-substituted pyridones), isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like. The term “heteroaryl” also refers to partially saturated heteroaryl moieties such as, for example, tetrahydroisoquinolyl, tetrahydroquinolyl and the like. In one embodiment, a heteroaryl has from 5 to 7 ring atoms. In another embodiment, a heteroaryl has 5 or 6 ring atoms. In another embodiment, a heteroaryl has 5 ring atoms. In still another embodiment, a heteroaryl has 6 ring atoms.

The term “heterocycloalkyl,” as used herein, refers to a non-aromatic, saturated monocyclic or multicyclic ring system comprising from 3 to about 10 ring atoms, wherein from 1 to 4 of the ring atoms are independently O, S or N and the remainder of the ring atoms are carbon atoms. In one embodiment, a heterocycloalkyl group has from about 5 to about 10 ring atoms. In another embodiment, a heterocycloalkyl group has 5 or 6 ring atoms. There are no adjacent oxygen and/or sulfur atoms present in the ring system. Any —NH group in a heterocycloalkyl ring may exist protected such as, for example, as an —N(Boc), —N(CBz), —N(Tos) group and the like; such protected heterocycloalkyl groups are considered part of this invention. A heterocycloalkyl group can be optionally substituted by one or more “ring system substituents” which may be the same or different, and are as defined herein below. The nitrogen or sulfur atom of the heterocyclyl can be optionally oxidized to the corresponding N-oxide, S-oxide or S,S-dioxide. Non-limiting examples of illustrative monocyclic heterocycloalkyl rings include piperidyl, pyrrolidinyl, piperazinyl, morpholinyl, thiomorpholinyl, thiazolidinyl, 1,4-dioxanyl, tetrahydrofuranyl, tetrahydrothiophenyl, lactam, lactone, and the like. A ring carbon atom of a heterocycloalkyl group may be functionalized as a carbonyl group. An illustrative example of such a heterocycloalkyl group is is pyrrolidonyl:

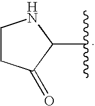

The symbol

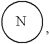

when present inside a ring, indicates that one of the ring's non-fused carbon atoms is replaced with a nitrogen atom. For example, in the structure:

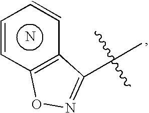

the presence of the symbol

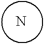

inside the 6-membered ring indicates that a nitrogen atom that is located at one of the 4 non-fused positions of the 6-membered ring, i.e., positions 1, 2, 3 or 4 indicated below:

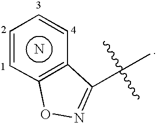

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound’ or “stable structure” is meant a compound that is sufficiently robust to survive isolation to a useful degree of purity from a reaction mixture, and formulation into an efficacious therapeutic agent.

The term “ring system substituent,” as used herein, refers to a substituent group attached to an aromatic or non-aromatic ring system which, for example, replaces an available hydrogen on the ring system. Ring system substituents may be the same or different, each being independently selected from the group consisting of alkyl, alkenyl, alkynyl, aryl, heteroaryl, arylalkyl, alkylaryl, heteroarylalkyl, heteroarylalkenyl, heteroarylalkynyl, alkylheteroaryl, —OH, hydroxyalkyl, —O-alkyl, -alkylene-O-alkyl, —O-aryl, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylthio, arylthio, heteroarylthio, arylalkylthio, heteroarylalkylthio, cycloalkyl, heterocyclyl, —O—C(O)-alkyl, —O—C(O)-aryl, —O—C(O)-cycloalkyl, —C(═N—CN)—NH₂, —C(═NH)—NH₂, —C(═NH)—NH(alkyl), Y₁Y₂N—, Y₁Y₂N-alkyl-, Y₁Y₂NC(O)— and Y₁Y₂NSO₂—, wherein Y₁ and Y₂ can be the same or different and are independently selected from the group consisting of hydrogen, alkyl, aryl, cycloalkyl, and arylalkyl. “Ring system substituent” may also mean a single moiety which simultaneously replaces two available hydrogens on two adjacent carbon atoms (one H on each carbon) on a ring system. Examples of such moiety are methylene dioxy, ethylenedioxy, —C(CH₃)₂— and the like which form moieties such as, for example:

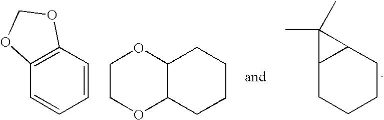

Any atom with unsatisfied valences in the text, schemes, examples and tables herein is assumed to have the sufficient number of hydrogen atom(s) to satisfy the valences.

The term “one or more Compounds of Formula (I)” as used herein in connection with the treatment or prevention of a Condition in a patient means that at least one Compound of Formula (I) is administered to the patient. In one embodiment, the phrase “one or more” refers to one Compound of Formula (I). In another embodiment, the phrase “one or more” refers to two Compounds of Formula (I).

The term “coxib” as used herein, refers to an agent that is an inhibitor of the COX-2 enzyme. A coxib may inhibit both the COX-1 and COX-2 enzymes, or may selectively inhibit the COX-2 enzyme.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in organic Synthesis (1991), Wiley, New York.

When any variable (e.g., aryl, heterocycle, R², etc.) occurs more than one time in any constituent or in Formula (I), its definition on each occurrence is independent of its definition at every other occurrence.

As used herein, the term “composition” is intended to encompass a product comprising the specified ingredients in the specified amounts, as well as any product which results, directly or indirectly, from combination of the specified ingredients in the specified amounts.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g, a drug precursor) that is transformed in vivo to yield a Compound of Formula (I) or a pharmaceutically acceptable salt, hydrate or solvate of the compound. The transformation may occur by various mechanisms (e.g., by metabolic or chemical processes), such as, for example, through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, if a Compound of Formula (I) or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl, and the like.

Similarly, if a Compound of Formula (I) contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a —OH group of the hemiacetal form of a carbohydrate), and the like.

If a Compound of Formula (I) incorporates an amine functional group, a prodrug can be formed by the replacement of a hydrogen atom in the amine group with a group such as, for example, R-carbonyl, RO-carbonyl, NRR′-carbonyl where R and R′ are each independently (C₁-C₁₀)alkyl, (C₃-C₇)cycloalkyl, benzyl, or R-carbonyl is a natural α-aminoacyl or natural α-aminoacyl, —C(OH)C(O)OY¹ wherein Y¹ is H, (C₁-C₆)alkyl or benzyl, —C(OY²)Y³ wherein Y² is (C₁-C₄)alkyl and Y³ is (C₁-C₆)alkyl, carboxy(C₁-C₆)alkyl, amino(C₁-C₄)alkyl or mono-N— or di-N,N—(C₁-C₆)alkylaminoalkyl, —C(Y⁴)Y⁵ wherein Y⁴ is H or methyl and Y⁵ is mono-N— or di-N,N—(C₁-C₆)alkylamino morpholino, piperidin-1-yl or pyrrolidin-1-yl, and the like.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of illustrative solvates include ethanolates, methanolates, and the like. “Hydrate” is a solvate wherein the solvent molecule is H₂O.

One or more compounds of the invention may optionally be converted to a solvate. Preparation of solvates is generally known. Thus, for example, M. Caira et al, J. Pharmaceutical Sci.; 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS Pharm Sci Tech., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

The Compounds of Formula (I) can form salts which are also within the scope of this invention. Reference to a Compound of Formula (I) herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a Compound of Formula (I) contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the Formula (I) may be formed, for example, by reacting a Compound of Formula (I) with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), arylalkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Pharmaceutically acceptable esters of the present compounds include the following groups: (1) carboxylic acid esters obtained by esterification of the —OH groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), arylalkyl (for example, benzyl), aryloxyalkyl (for example, phenoxymethyl), aryl (for example, phenyl optionally substituted with, for example, halo, C₁₋₄alkyl, or C₁₋₄alkoxy or amino); (2) sulfonate esters, such as alkyl- or arylalkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di(C₆₋₂₄)acyl glycerol.

Compound of Formula (I), and salts, solvates, hydrates, esters and prodrugs thereof, may exist in their tautomeric form (for example, as an amide or imino ether, or in keto-enol form). All such tautomeric forms are considered equivalent and are contemplated herein as part of the present invention.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the Compounds of Formula (I) may be atropisomers (e.g., substituted biaryls) and are considered as part of this invention. Enantiomers can also be separated by use of chiral HPLC column.

All stereoisomers (for example, geometric isomers, optical isomers and the like) of the present compounds (including those of the salts, solvates, hydrates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). (For example, if a Compound of Formula (I) incorporates a double bond or a fused ring, both the cis- and trans-forms, as well as mixtures, are embraced within the scope of the invention. Also, for example, all keto-enol and imine-enamine forms of the compounds are included in the invention.).

Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to equally apply to the salt, solvate, ester and prodrug of enantiomers, stereoisomers, rotamers, tautomers, positional isomers, racemates or prodrugs of the inventive compounds.

The present invention also embraces isotopically-labelled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labelled Compounds of Formula (I) (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labelled Compounds of Formula (I) can generally be prepared using synthetic chemical procedures analogous to those disclosed herein for making the Compounds of Formula (I), by substituting an appropriate isotopically labelled starting material or reagent for a non-isotopically labelleds starting material or reagent.

Polymorphic forms of the Compound of Formula (I), and of the salts, solvates, hydrates, esters and prodrugs of the Compound of Formula (I), are intended to be included in the present invention.

The compounds of this invention can be ligands for the histamine H₃ receptor. In one embodiment, the Compounds of Formula (I) are antagonists of the H₃ receptor.

The following abbreviations are used herein and have the following meanings: AcOH is acetic acid; t-BOC is t-butyloxycarbonyl; Ci/mmol is curie/mmol (a measure of specific activity); m-CPBA is m-chloroperbenzoic acid; CSA camphorsulfonic acid; CBZ is carbonylbenzyloxy (—C(O)OCH₂C₆H₅); DBU is 1,8-diazabicyclo[5.4.0]undec-7-ene; DBN is 1,5-diazabicyclo[4.3.0]non-5-ene; DCC is dicyclohexylcarbodiimide; Dibal-H is diisobutylaluminum hydride; DIPEA is N,N-diisopropylethylamine; DMAP is 4-(dimethylamino)pyridine; DEC is 2-diethylaminoethyl chloride hydrochloride; DMF is N,N-dimethylformamide; EDCI is 1-(3-dimethylaminopropyl)-3-ethylcarbodiimide; EtOAc is ethyl acetate; EtOH is ethanol; FMOC is 9-fluorenylmethoxycarbonyl; HOBT is 1-hydroxybenzotriazole; HPLC is high performance liquid chromatography; HRMS is high resolution mass spectrometry; Ki is inhibition constant for substrate/receptor complex; LAH-lithium aluminum hydride; LDA is lithium diisoprpylamide; LRMS is low resolution mass spectrometry; MeOH is methanol; NaBH(OAc)₃ is sodium triacetoxyborohydride; NaBH₄ is sodium borohydride; NaBH₃CN is sodium cyanoborohydride; NaHMDS is sodium hexamethyl disilazide; pA2 is −log EC₅₀, as defined by J. Hey, Eur. J. Pharmacol., (1995), Vol. 294, 329-335; PCC is pyridinium chlorochromate; PyBOP is benzotriazole-1-yl-oxy-trispyrrolidino-phosphonium hexaflurophosphate; TEMPO is 2,2,6,6-tetramethyl-1-piperidinyloxy, free radical; TFA is trifluoroacetic acid; TMAD is N,N,N′,N′-tetramethylazodicarboxamide; TMEDA is tetramethylethylenediamine; Tr is triphenylmethyl; Tris is tris(hydroxymethyl)aminomethane; and p-TsOH is p-toluenesulfonic acid.

### The Compounds of Formula (I)

The present invention provides uses of, and compositions comprising, compounds having the formula:

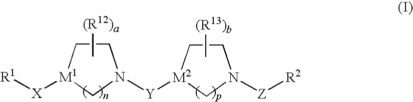

and pharmaceutically acceptable salts, solvates, esters and prodrugs thereof, wherein R¹, R², R¹², R¹³, M¹, M², X, Y, Z, a, b, n and p are defined above for the Compounds of Formula (I).

In one embodiment, R¹ is unsubstituted aryl.

In another embodiment, R¹ is aryl that is substituted with from 1 to 3 substituents independently selected from halo, alkyl or haloalkyl;

In another embodiment, R¹ is heteroaryl.

In still another embodiment, R¹ is heteroaryl that is substituted with from 1 to 3 substituents independently selected from halo, alkyl or haloalkyl.

In a further embodiment, R¹ is taken together with X to form:

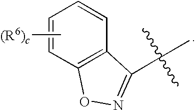

In one embodiment, R¹ is phenyl.

In another embodiment, R¹ is phenyl substituted with from 1-3 groups independently selected from —F, —Cl or —CF₃.

In another embodiment, R¹ is phenyl substituted with a branched alkyl group.

In still another embodiment, R¹ is phenyl substituted with a linear alkyl group.

In yet another embodiment, R¹ is phenyl substituted with a haloalkyl group.

In one embodiment, R¹ is a five or six membered heteroaryl.

In another embodiment, R¹ is a six membered heteroaryl ring.

In another embodiment, R¹ is pyridyl, thienyl, pyrimidinyl, thiazolyl or pyridyl N-oxide.

In one embodiment, R¹ is pyridyl.

In still another embodiment, R¹ is:

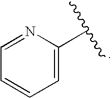

In a further embodiment, R¹ is heteroaryl, substituted with a halo-substituted or an alkyl-substituted heteroaryl group.

In one embodiment R¹ is halopyridyl or alkylthiazolyl.

In another embodiment, R¹ is:

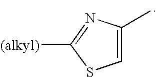

In a further embodiment, R¹ is:

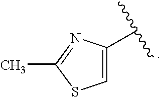

In another embodiment, R¹ is:

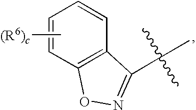

wherein R⁶ is fluoro and c is 1.

In one embodiment, X is —C(NOR³)—.

In another embodiment, X is —C(NO(alkyl))-.

In another embodiment, X is —C(NOCH₃)—.

In still another embodiment, X is —C(O)—.

In one embodiment, M¹ is CH.

In another embodiment, M¹ is N.

In one embodiment, M² is CH.

In another embodiment, M² is CF.

In another embodiment, M² is N.

In another embodiment, M¹ and M² are each CH.

In still another embodiment, M¹ and M² are each N.

In another embodiment, M¹ is N and M² is CH.

In a further embodiment, M¹ is CH and M² is N.

In one embodiment, n is 2.

In another embodiment, a is 0 or 1

In another embodiment, a is 0.

In another embodiment, b is 0 or 1

In still another embodiment, b is 0.

In yet another embodiment, c is 0 or 1

In another embodiment, c is 0.

In a further embodiment, c is 1 and R⁶ is fluoro.

In one embodiment, e is 1-5.

In one embodiment, Y is —C(O)—.

In another embodiment, Y is —CH₂—.

In another embodiment, Y is —C(S)—.

In one embodiment, p is 2.

In one embodiment, Z is C₁-C₃ alkyl.

In another embodiment, Z is —CH₂—.

In another embodiment, Z is —CH(CH₃)—.

In one embodiment, R² is a six membered heteroaryl.

In another embodiment, R² is pyridyl.

In another embodiment, R² is pyrimidinyl.

In another embodiment, R² is pyridyl substituted with —NR⁴R⁵.

In still another embodiment, R² is pyrimidinyl substituted with —NR⁴R⁵.

In yet another embodiment, R² is pyridyl substituted with —NH₂.

In one another embodiment, R² is pyrimidinyl substituted with —NH₂.

In a further embodiment, R² is:

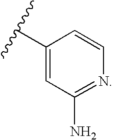

In another embodiment, R² is:

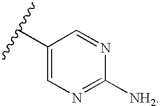

In one embodiment, R³ is H.

In another embodiment, R³ is alkyl.

In another embodiment, R³ is methyl.

In one embodiment, R⁴ is H.

In another embodiment, R⁴ is lower alkyl.

In another embodiment, R⁴ is methyl.

In one embodiment, R⁵ is H.

In another embodiment, R⁵ is lower alkyl.

In another embodiment, R⁵ is —C(O)R⁴.

In still another embodiment, R⁵ is methyl.

In one embodiment, R¹² is alkyl.

In another embodiment, R¹² is halo.

In another embodiment, R¹² is —OH.

In still another embodiment, R¹² is H.

In yet another embodiment, R¹² is —F.

In one embodiment, R¹³ is alkyl.

In another embodiment, R¹³ is halo.

In another embodiment, R¹³ is —OH.

In still another embodiment, R¹³ is H.

In yet another embodiment, R¹³ is —F.

In one embodiment, the Compounds of Formula (I) have the formula (Ia):

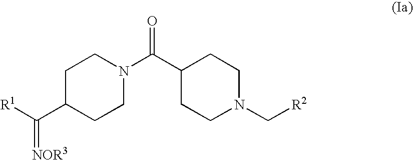

wherein R¹, R² and R³ are as defined above for the Compounds of Formula (I).

In one embodiment, R¹ is heteroaryl.

In another embodiment, R¹ is pyridyl.

In another embodiment, R¹ is 2-pyridyl.

In still another embodiment, R¹ is:

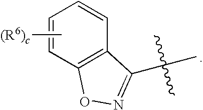

In one embodiment, R² is six-membered heteroaryl.

In another embodiment, R² is:

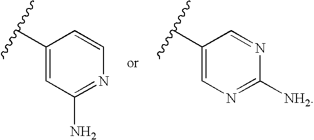

In another embodiment, R³ is H or alkyl.

In another embodiment, R³ is alkyl.

In still another embodiment, R³ is methyl.

In another embodiment, R¹ is heteroaryl and R² is six-membered heteroaryl.

In another embodiment, R¹ is heteroaryl and R³ is H or alkyl

In one embodiment, R¹ is 2-pyridyl or

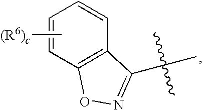

and R³ is alkyl.

In another embodiment, R² is:

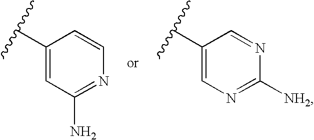

and R³ is alkyl.

In a further embodiment, R¹ is 2-pyridyl or

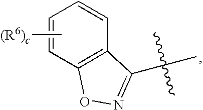

R² is six-membered heteroaryl, and R³ is alkyl.

In yet another embodiment, R¹ heteroaryl, R² is:

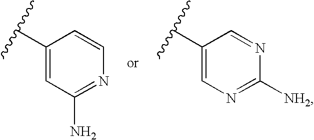

and R³ is alkyl

In a further embodiment, R¹ is 2-pyridyl or

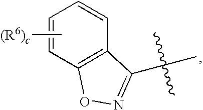

### R² is:

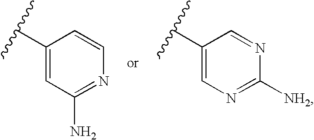

and R³ is alkyl.

In yet another embodiment, R¹ is 2-pyridyl or

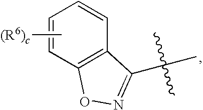

### R² is:

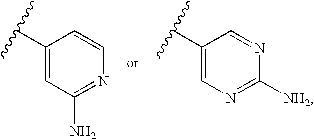

and R³ is methyl.

Illustrative examples of the Compounds of Formula (I) are found in the Examples below, and in Tables 1, 2 and 3 below.

In one embodiment, the Compound of Formula (I) is Compound 32 or a pharmaceutically acceptable salt, solvate, ester or prodrug thereof.

In another embodiment, the Compound of Formula (I) is Compound 54 or a pharmaceutically acceptable salt, solvate, ester or prodrug thereof.

In another embodiment, the Compound of Formula (I) is Compound 55 or a pharmaceutically acceptable salt, solvate, ester or prodrug thereof.

In still another embodiment, the Compound of Formula (I) is Compound 253A or a pharmaceutically acceptable salt, solvate, ester or prodrug thereof.

In yet another embodiment, the Compound of Formula (I) is Compound 287 or a pharmaceutically acceptable salt, solvate, ester or prodrug thereof.

In another embodiment, the Compound of Formula (I) is Compound 320 or a pharmaceutically acceptable salt, solvate, ester or prodrug thereof.

In a further embodiment, the Compound of Formula (I) is Compound 446 or a pharmaceutically acceptable salt, solvate, ester or prodrug thereof.

In one embodiment, the Compound of Formula (I) is in isolated or purified form.

In another embodiment, for the Compounds of Formula (I), variables R¹, R², R¹², R¹³, M¹, M², X, Y, Z, a, b, n and p are selected independently of each other.

### Methods for Making the Compounds of Formula (I)

Methods useful for making the Compounds of Formula (I) are set forth in the Examples below and generalized in Schemes 1-6.

Scheme 1 illustrates methods useful for making the compounds of formulas 8 and 9, which are useful intermediates for making the Compounds of Formula (I).

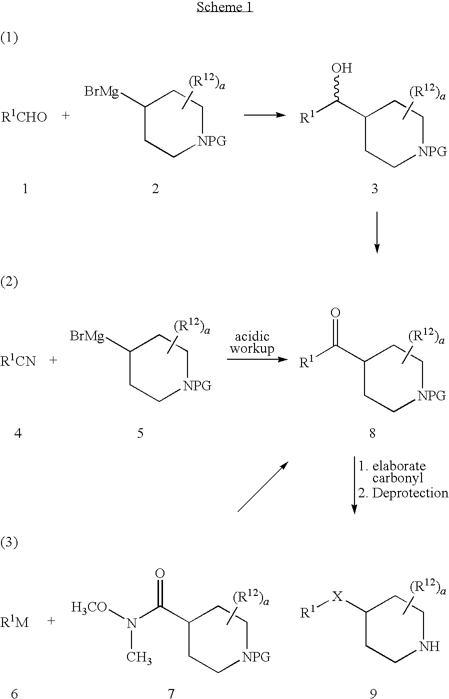

Wherein R¹, R¹², X and a are as defined above for the Compounds of Formula (I), PG is a nitrogen protecting group (such as BOC, CBz, FMOC, methyl or benzyl), and M is Li, MgCl, MgBr or MgI.

A Grignard reagent of formula 2 can be reacted with an aldehyde of formula 1 to provide hydroxy compound of formula 3, which can then be oxidized to provide the compounds of formula 8. Alternatively a Grignard reagent of formula 2 can be reacted with a nitrile of formula 4 which, upon acidic workup, provides the compounds of formula 8 directly. In another alternative procedure, an amide of formula 7 can be reacted with an organometallic reagent of formula 6 to directly provide the compounds of formula 8. The carbonyl group of a compound of formula 8 can then be optionally further elaborated to provide compounds wherein X is other than carbonyl, after which the amine protecting group can be removed to provide the intermediate compounds of formula 9.

Scheme 2 illustrates a method useful for making the compounds of formula 12, which are useful intermediates for making the Compounds of Formula (I).

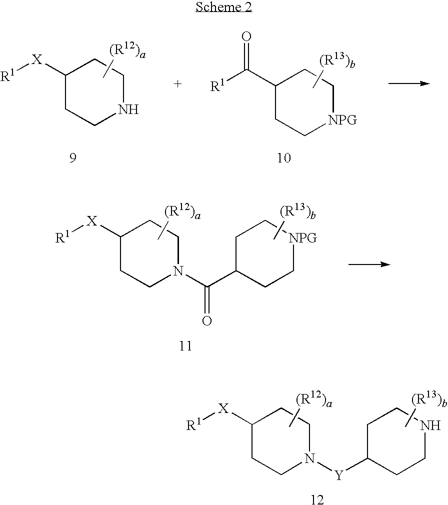

Wherein R¹, R¹², R¹³, X, Y, a and b are as defined above for the Compounds of Formula (I), and PG is a nitrogen protecting group (such as BOC, CBz, FMOC, methyl or benzyl).

An amine of formula 9 can be coupled with a compound of formula 10, wherein R′ is —OH, —Cl or —OC(O)-alkyl, using coupling methods well known in the art of organic synthesis to provide the compounds of formula 11. The carbonyl group of a compound of formula 11 can then be optionally further elaborated to provide compounds wherein Y is other than carbonyl, after which the amine protecting group can be removed to provide the intermediate compounds of formula 12.

Scheme 3 illustrates a method useful for making the compounds of formula 14, which correspond to the Compounds of Formula (I).

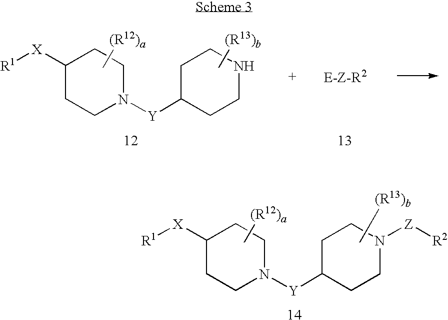

Wherein R¹, R², R¹², R¹³, X, Y, Z, a and b are as defined above for the Compounds of Formula (I) and E is —C(O)— or a leaving group, such as —Cl, —Br, —I, —O-mesyl, —O-tosyl, or —O-triflyl.

The free piperidine nitrogen atom of a compound of formula 12 can be alkylated using a compound of formula 13 to provide the intermediate compounds of formula 14. When E is a carbonyl group, the imine formed must be reduced using a reducing agent such as NaBH(OAc) to provide the compounds of formula 14, which correspond to the Compounds of Formula (I), wherein Z is methylene. Alternatively, when E is a leaving group such as a halo, mesylate, tosylate or triflate, compounds 12 and 13 can be reacted in the presence of a tertiary amine base to provide the compounds of formula 14 directly.

Scheme 4 illustrates a method useful for making the compounds of formula 16, which correspond to the Compounds of Formula (I), wherein Y is an oxime.

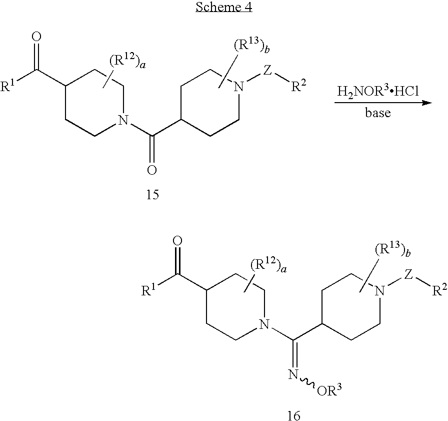

Wherein R¹, R², R³, R¹², R¹³, X, Z, a and b are as defined above for the Compounds of Formula (I).

Compound 15 (which is the compound of formula 14, wherein Y is —C(O)—) can be reacted with H₂NOR³.HCl in a base, such as pyridine, to provide the compounds of formula 16, which correspond to the Compounds of Formula (I), wherein Y is an oxime. Alternatively, a compound of formula 15 can be reacted with H₂NOR³.HCl in an alcoholic solvent in the presence of a base, such as, NaOAc, to provide the compounds of formula 16.

An alternate approach to the synthesis of compounds of Formula (I) involves the synthesis of the two halves of the molecule followed by coupling of the two pieces, i.e.,

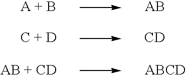

In this case, the synthesis of the AB fragment (Compound 9) is the same as that described above. The synthesis of the CD fragment (compound 18) is set forth below in Scheme 5.

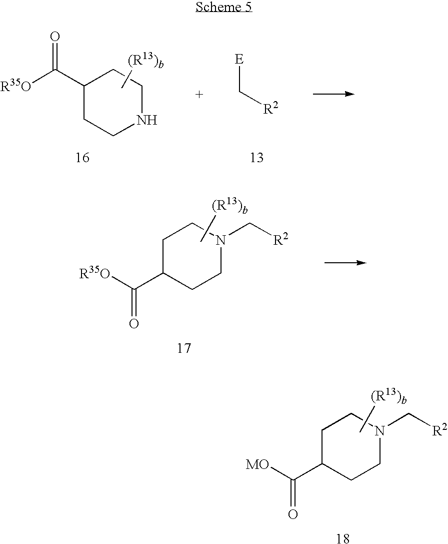

Wherein R², R¹³ and b are as defined above for the Compounds of Formula (I); R³⁵ is methyl or ethyl; E is a leaving group; and M is Li, Na, or K.

A compound of formula 17 (prepared by reacting a compound of formula 16 and a compound of formula 13 using the method described above for the synthesis of compound 14) can be saponified in a mixed solvent, such as, for example: (1) EtOH or MeOH and water, or (2) THF, water, and MeOH, using an alkali metal base such as LiOH or NaOH to provide a compound of formula 18. A compound of formula 18 can then combined with a compound of formula 9, as described above, to provide the intermediate compounds of formula 14. The remaining steps in the synthetic method are then are the same.

It is to be noted that the Compounds of Formula (I) can be made using the methodology set forth above in Schemes 1-5 in any order which will provide the Compounds of Formula (I). Although schemes 1-4 present the synthesis of the Compounds of Formula (I) in a linear fashion, it will be apparant to one skilled in the art of organic synthesis that the above methods may also be used in a convergent fashion to make the compounds of the invention.

Scheme 6 shows an alternative method useful for making the Compounds of Formula (I), wherein X is —C(═NOH)— or —C(═NO-alkyl)-.

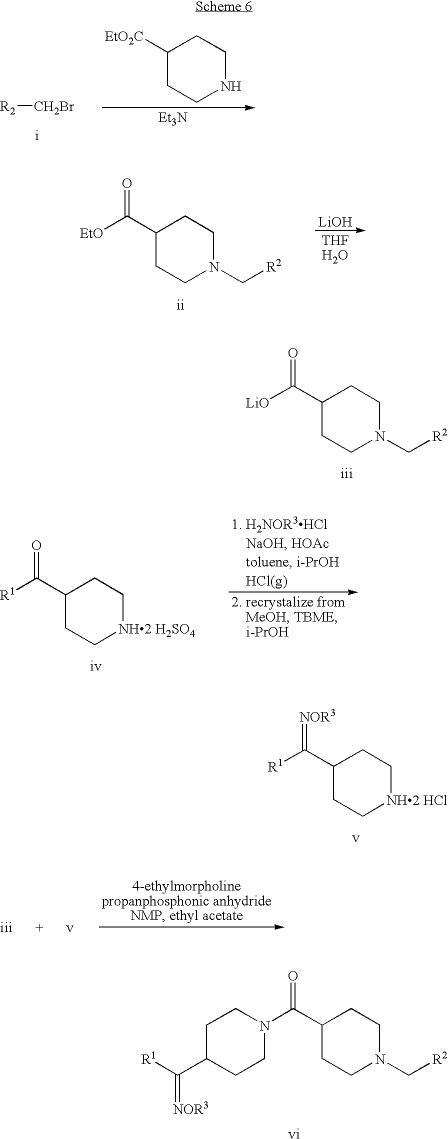

Wherein R¹, R² are as defined above for the Compounds of Formula (I), and R³ is H or alkyl.

A bromomethyl compound of formula i can be reacted with in the presence of triethylamine to provide the piperidine compounds of formula ii. The ester moiety of a compound of formula ii can then be saponified using an alkali metal hydroxide, such as LiOH, for example, to provide the metal carboxylate compounds of formula iii.

In a separate reaction sequence, a compound of formula iv can be reacted with an alkoxylamine hydrochloride to provide the oxime compounds of formula v as a dihydrochloride salt. A compound of formula v can then be reacted with a compound of formula iii in the presence of 4-ethylmorpholine and propanphosphonic anhydride to provide the compounds of formula vi, which correspond to the Compounds of Formula (I), wherein X X is —C(═NOH)— or —C(═NO-alkyl)-.

### Examples

The following examples exemplify illustrative examples of compounds of the present invention and are not to be construed as limiting the scope of the disclosure. Alternative mechanistic pathways and analogous structures within the scope of the invention may be apparent to those skilled in the art.

### General Methods

The starting materials and reagents used in preparing compounds described are either available from commercial suppliers such as Aldrich Chemical Co. (Wisconsin, USA) and Acros Organics Co. (New Jersey, USA) or were prepared using methods well-known to those skilled in the art of organic synthesis. All commercially purchased solvents and reagents were used as received. LCMS analysis was performed using an Applied Biosystems API-100 mass spectrometer equipped with a Shimadzu SCL-10A LC column: Altech platinum C18, 3 um, 33 mm×7 mm ID; gradient flow: 0 minutes, 10% CH₃CN; 5 minutes, 95% CH₃CN; 7 minutes, 95% CH₃CN; 7.5 minutes, 10% CH₃CN; 9 minutes, stop. Flash column chromatography was performed using Selecto Scientific flash silica gel, 32-63 mesh. Analytical and preparative TLC was performed using Analtech Silica gel GF plates. Chiral HPLC was performed using a Varian PrepStar system equipped with a Chiralpak OD column (Chiral Technologies).

### Example 1

### Synthesis of Intermediate Compound 5A

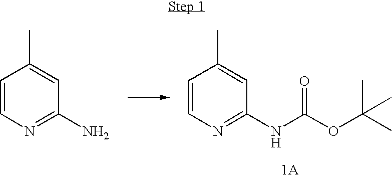

To a solution of 10.81 g (100 mmol) of 2-amino-4-methylpyridine in 250 ml of tert-butanol was added 26.19 g (120 mmol) of BOC anhydride. Reaction mixture was stirred at room temperature overnight, concentrated—dry loaded on silica gel and flash chromatographed (from 30% hexanes/CH₂Cl₂ to 0-2% acetone/CH₂Cl₂) to produce 15.25 g (73.32 mmol; 73%) of 1A as a white solid.

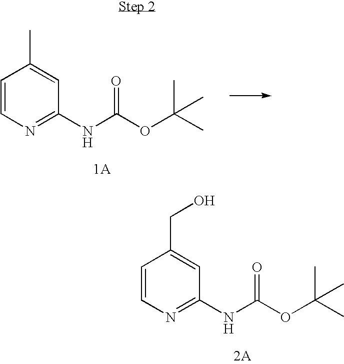

To a −78° C. solution of of 1A (35.96 g, 173 mmol) in of THF (1.4 L) was added of 1.4 M BuLi solution (272 ml, 381 mmol) in hexanes in portions over 30 min. Reaction mixture was then allowed to warm up and was stirred for 2 h at room then temperature, which resulted in the formation of an orange precipiate. The mixture was cooled back to −78° C., and predried oxygen (passed through a Drierite column) was bubbled through the suspension for 6 h while the temperature was maintained at −78° C. Reaction mixture color changed to yellow during this time. It was then quenched at −78° C. with 51.4 ml (700 mmol) of Me₂S followed by 22 ml (384 mmol) of AcOH. Reaction mixture was allowed to warm up and was stirred for 48 h at room temperature. Dilution with water and extraction with EtOAc were followed by concentration and flash chromatography (0-15% acetone/CH₂Cl₂) to provide 20.15 g (90 mmol; 52%) of alcohol 2A as a pale yellow solid.

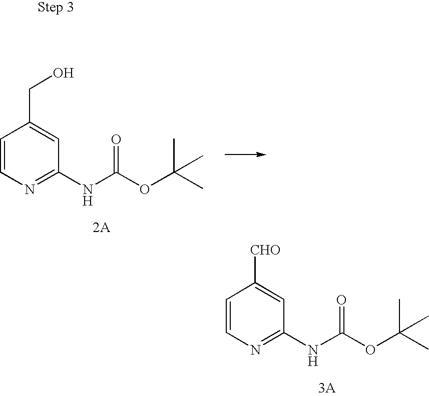

To a solution of 19.15 g (85.5 mmol) of alcohol 2A in 640 ml of CH₂Cl₂ was added saturated aqueous solution of 8.62 g (103 mmol) of NaHCO₃ and 444 mg (4.3 mmol) of NaBr. Reaction mixture was cooled to 0° C., and 140 mg (0.90 mmol) of TEMPO was introduced. Upon vigorous stirring 122 ml of 0.7 M (85.4 mmol) commercial bleach solution (5.25% in NaOCl) was added in portions over 40 min. After additional 20 min at 0° C. reaction mixture was quenched with saturated aqueous Na₂S₂O₃ and allowed to warm to room temperature. Dilution with water and extraction with CH₂Cl₂ were followed by concentration and flash chromatography (from 30% hexanes/CH₂Cl₂ to 0-2% acetone/CH₂Cl₂) to afford 15.97 g (71.9 mmol; 84%) of aldehyde 3A as an off-white solid.

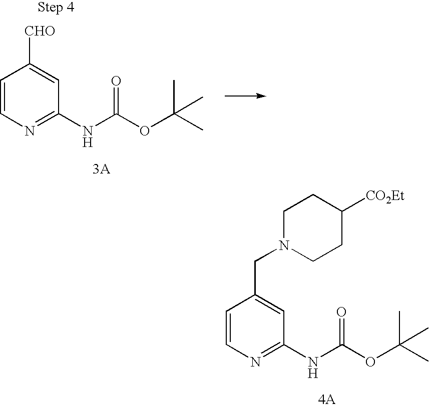

To a solution of 11.87 g (53.5 mmol) of aldehyde 3A in 370 ml of CH₂Cl₂ was added 9.07 ml (58.8 mmol) of ethyl isonipecotate followed by four drops of AcOH. Reaction mixture was then stirred for 40 min at room temperature after which 22.68 g (107 mmol) of NaBH(OAc)₃ was introduced. Reaction mixture was stirred overnight at room temperature, neutralized with saturated aqueous NaHCO₃, diluted with water and extracted with CH₂Cl₂. Concentration and flash chromatography (0-4% sat. NH₃ in MeOH/CH₂Cl₂) provided 19.09 mg (52.6 mmol; 98%) of 4A as an off-white solid.

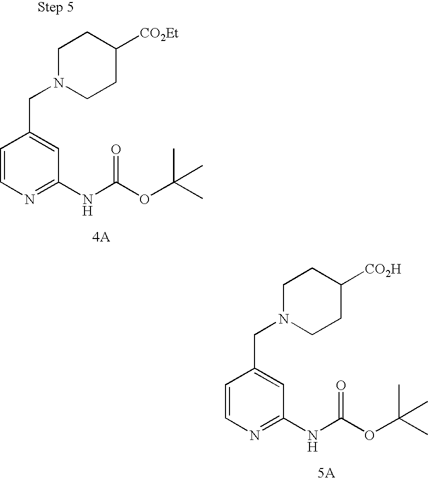

To a solution of 1.57 g (4.33 mmol) of ester 4A in 10 ml of a 3:1:1 mixture of THF-water-methanol was added 0.125 g (5.21 mmol) of LiOH. Reaction mixture was stirred overnight at room temperature, concentrated and exposed to high vacuum to obtain 1.59 g of crude acid 5A as a yellowish solid which was used without purification.

### Example 2

### Synthesis of Intermediate Compound 7A

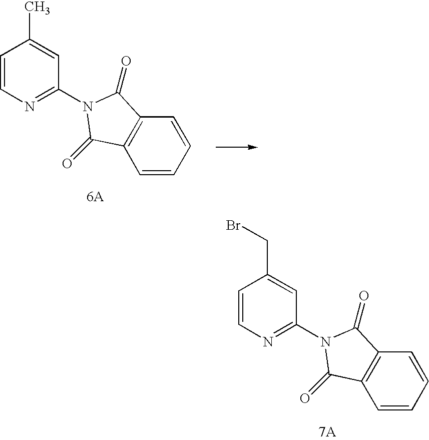

A solution of compound 6A (42 mmol), NBS (126 mmol) and Bz₂O₂ (4.2 mmol) in CCl₄ (400 ml) was refluxed at 80° C. for 5 h, cooled and stirred at room temperature overnight. The reaction was filtered and concentrated, and the residue was purified by flash column (30% EtOAc/Hexane) to obtain the desired compound 7A (3.1 g, 23%).

### Example 3

### Synthesis of Intermediate Compound 11A

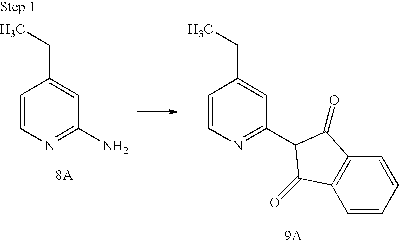

To a solution of 8A (10 g, 79.4 mmol) and DMAP (0.029 g, 0.24 mmol) in methylene chloride (150 mL) at 0° C. was added phthaloyl dichloride (16.1 g, 79.4 mmol) dropwise. The reaction mixture was stirred at room temperature overnight. After stirring overnight, the reaction was washed with saturated aqueous NaHCO₃, water, dried and concentrated to provide compound 9A as a yellow solid (20 g, 99.8%) which was used without further purification.

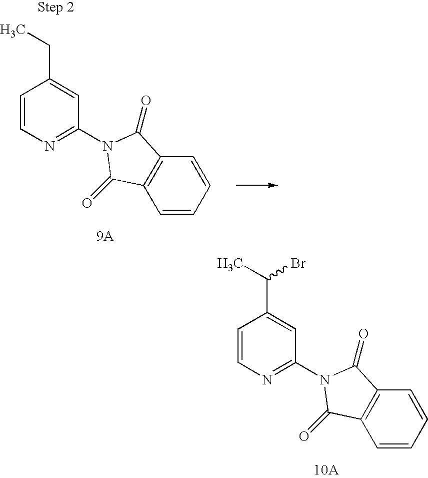

In a manner similar to that described in Example 2, compound 9A (20 g, 79.3 mmol) was converted to compound 9A.

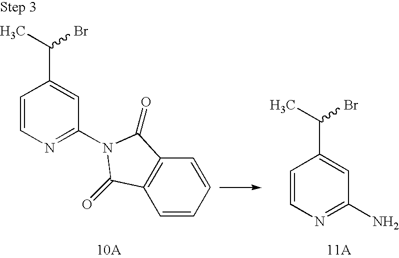

Compound 10A (0.5 g, 1.5 mmol) and hydrazine (0.5 M in ethanol, 5 mL, 2.5 mmol) were combined and stirred at room temperature overnight. The reaction was diluted with water and extracted with methylene chloride. The organic layer was dried, concentrated and the residue purified on a flash column (3% methanol in ethyl acetate) to provide compound 11A (0.2 g, 66%).

### Example 4

### Synthesis of Intermediate Compound 15A

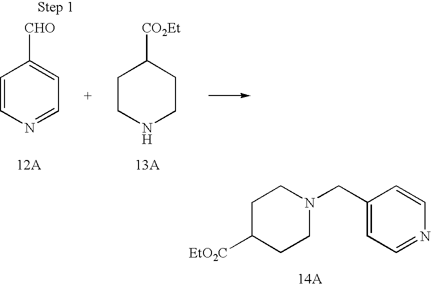

Compounds 12A (2 g, 18.3 mmol) and 13A (3.5 g, 22 mmol) were dissolved in methylene chloride and stirred at room temperature for 1 h. Na(OAc)₃BH (5.4 g, 25.6 mmol) was added and the mixture stirred at room temperature for 5 h. The reaction was washed with saturated aqueous NaHCO₃, dried and concentrated, and the residue purified by flash column (2% methanol in ethyl acetate). Compound 14A was obtained (4.5 g, 99%).

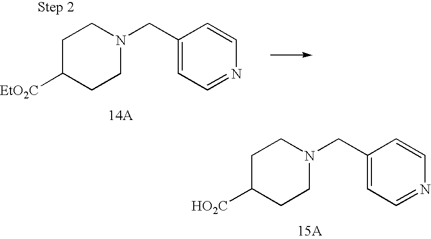

In a manner similar to that described in Example 1, Step 5, compound 14A (0.35 g, 1.4 mmol) was converted to compound 15A (0.31 g, 100%).

### Example 5

### Synthesis of Compound 23

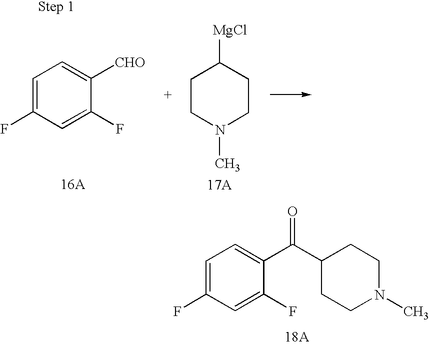

To the solution of 2,4-diflorobenzylaldehyde (16A, 28.1 mmol) in THF (10 ml) was added the Grignard reagent 17A (1.33M in THF, 30 ml), and the mixture was stirred at room temperature overnight. The reaction was quenched with saturated NH₄Cl (150 ml), extracted three times with EtOAc (100 ml), dried, filtered and concentrated. Flash chromatography (20% MeOH/EtOAc) yielded the desired compound 18A (1.8 g, 27%).

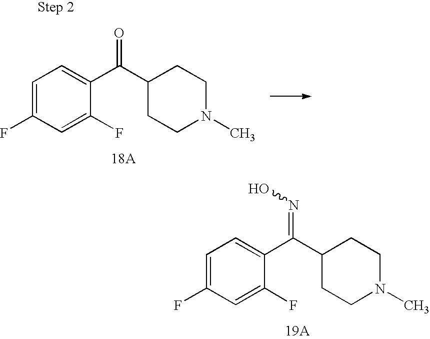

Compound 18A (1.6 g, 6.7 mmol), H₂NHOH.HCl (0.95 g, 6.7 mmol) and pyridine (10 mL) were combined and heated to 60° C. overnight. The pyridine was removed under vacuum and the residue treated with methylene chloride and saturated aqueous NaHCO₃. The organic layer was separated, dried, and concentrated, and the residue purified by flash chromatography to provide compound 19A (1.4 g, 82%).

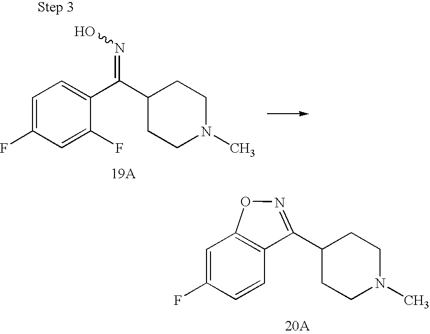

To the suspension of NaH (0.41 g, 10.2 mmol) in THF (10 ml) was slowly added a solution of 19A (1.3 g, 5.11 mmol) in DMF (5 ml) dropwise and the reaction stirred at 70˜75° C. overnight. The mixture was extracted twice with EtOAc and three times with H₂O (30 ml), dried over MgSO₄ and concentrated to provide crude 20A which was used without further purification (1.04 g, 87%).

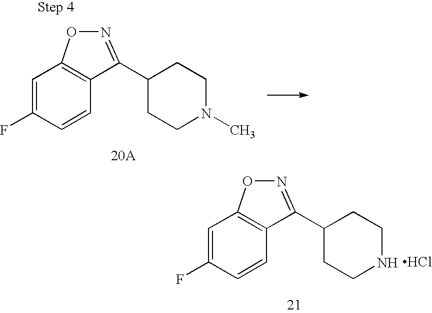

To the solution of compound 20A (4.3 mmol) in dichloroethane (20 ml) at 0° C. was added 2-chloroethyl chloroformate (6.2 mmol) and triethylamine (7.2 mmol) and the reaction was stirred at room temperature overnight. The solvent was evaporated, Et₂O was added to the residue, and the unreacted starting material was removed by filtration. The filtrate was concentrated and the residue redissolved in MeOH and refluxed for 30 min. Removal of the methanol gave the product 21 (0.3 g) which was used without further purification.

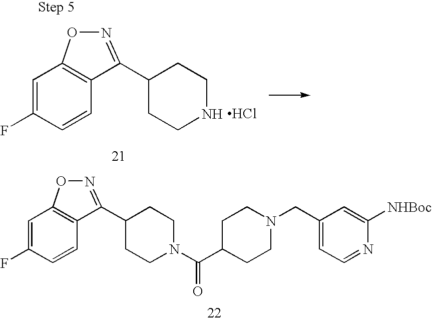

To a mixture of compound 21 (1.64 mmol), compound 5A (1.64 mmol) and PyBOP (1.64 mmol) was added DIPEA (4.92 mmol) and CH₂Cl₂ (10 ml), and the reaction was stirred over the weekend at room temperature. Saturated NaHCO₃ (100 ml) was added and the reaction was extracted and twice with CH₂Cl₂ (100 ml), dried over solid MgSO₄, concentrated and flash chromatographed (70% EtOAc/Hexane) to provide compound 22 (1.04 mmol, 64%).

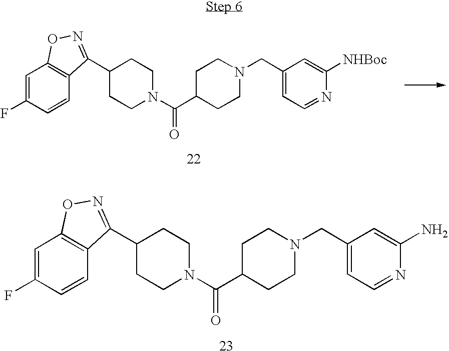

Compound 22 (0.2 g, 0.37 mmol) was dissolved in CF₃CO₂H (3 mL) and methylene chloride (3 mL) and stirred at room temperature overnight. The solvent was removed by evaporation, saturated aqueous NaHCO₃ was added and mixture extracted with methylene chloride. The organic layer was dried (MgSO₄), filtered and concentrated, and the residue purified by flash chromatography to provide compound 23 (0.11 g, 68%).

### Example 6

### Synthesis of Compounds 32 and 33

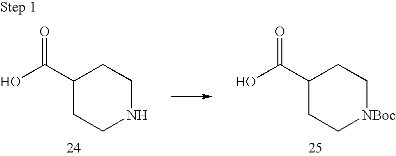

A solution of 24 (50 g, 387 mmol) and triethylamine (110 mL) in dioxane (400 mL) at 4° C. was treated with Boc₂O (93 g, 426 mmol). The cooling bath was removed and the solution allowed to warm to room temperature. After 21 h, the volume was reduced by two-thirds under vacuum. The residue was poured into ethyl acetate (250 mL) and water (250 mL). Saturated aqueous NaHCO₃ (250 mL) was added and the organic phase was separated and discarded. The aqueous phase was acidified with 10% HCl and extracted with ethyl acetate. The combined organic phases were washed with water, brine, and dried (Na₂SO₄), and concentrated to provide 25 as a white powder (82 g, 94%).

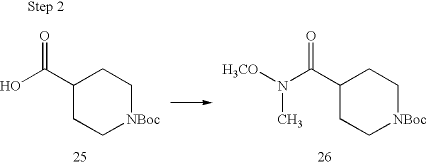

To a solution of compound 25 (40 g, 175 mmol) in DMF (250 mL) at 4° C. was added N,O-dimethyl-OHamine, hydrochloride (34 g), EDCI (44 g, 0.228 mol), HOBT (2.4 g), and DIPEA (120 mL). The reaction was warmed to room temperature and stirred overnight. The reaction was then concentrated to half volume in vacuo and poured onto 1:1 ethyl acetate:water. The organic layer was separated and the aqueous layer extracted with additional ethyl acetate. The combined organic layers were washed with saturated aqueous NH₄Cl, saturated aqueous NaHCO₃, water, and brine, and dried. Concentration gave 26 as a light yellow oil (46.7 g, 99%)

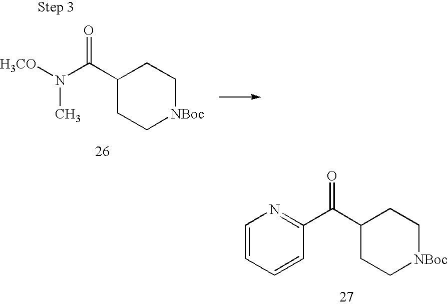

To a solution of 2-bromopyridine (17.6 mL, 0.184 mol) in THF (600 mL) at −78° C. was added n-BuLi (115 mL of a 1.6M solution in hexanes, 0.184 mol) dropwise over 15 min. After stirring for an additional 30 min at this temperature, a solution of 26 (25 g, 91.9 mmol) in THF (500 mL) was added dropwise over 15 min. The reaction was removed from the cold bath and placed in an oil bath and heated to 60° C. for 1.5 h. The reaction was then cooled to 4° C., diluted with ether (500 mL), and treated with saturated aqueous NaHCO₃ (□5 mL). The mixture was transferred to an Erlenmeyer flask and diluted with additional ether (700 mL). Additional saturated aqueous NaHCO₃ was added followed by solid NaHCO₃. The mixture was filtered through a plug of solid NaHCO₃ and concentrated in vacuo. Flash column chromatography (0-20% ethyl acetate in hexanes) yielded compound 27 as a yellow oil (16.85 g, 63%).

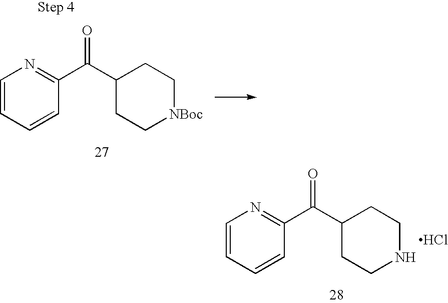

A solution of 27 (3.3 g, 11.4 mmol) in methanol (50 mL) was treated with 4M HCl in dioxane (50 mL) and stirred at room temperature for 1.5 h. Removal of the solvent in vacuo gave 28 as a tan powder (3 g, 100%)

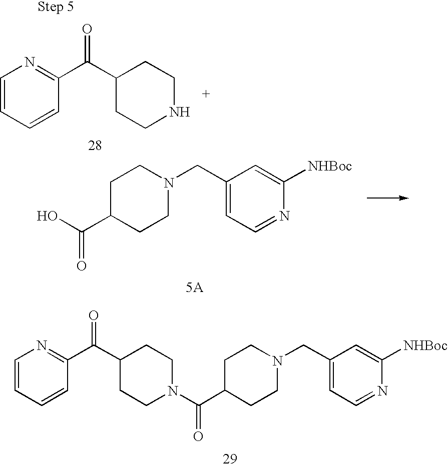

To a suspension of compound 5A (17.4 g, 50 mmol), compound 28 (11 g, 42 mmol), and diisopropylethylamine (34.6 mL, 199 mmol) in DMF (125 mL) was added HOBT (7.83 g, 58 mmol), EDC (18.54 g, 96.7 mmol), and 4 Å molecular sieves. The mixture was stirred for 40 h at room temperature, diluted with methylene chloride (600 mL) and 0.5 N NaOH (400 mL) and filtered. The precipitate was washed thoroughly with additional 0.5N NaOH and methylene chloride. The combined organic phases were concentrated and chromatographed twice on silica gel (1:1 hexane:methylene chloride to 6% saturated NH₃ in methanol in methylene chloride) to produce 29 as a tan solid (22.3 g) which was used as is in the next step.

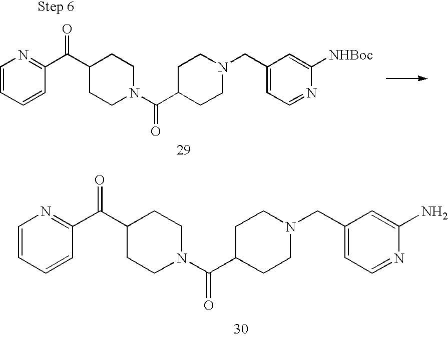

A solution of 29 (22.3 g, 44 mmol) in methylene chloride (120 mL) and trifluoroacetic acid (60 mL) was stirred for 7 h at room temperature. The reaction was concentrated, exposed to high vacuum for 3 h, dissolved in toluene and concentrated and then exposed again to high vacuum. The so-obtained crude brown oil was used in the next step without further purification.

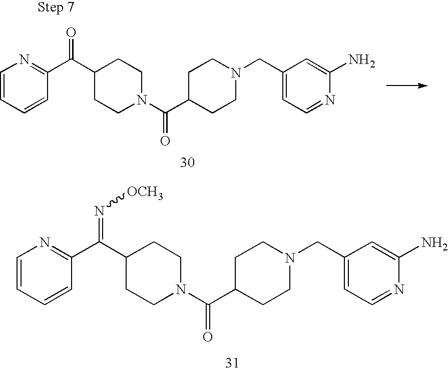

Compound 30 (□17.9 g, 44 mmol) was dissolved in pyridine (420 mL), treated with H₂NOCH₃.HCl (21.78 g, 264 mmol) and heated to 90° C. for 14 h. The reaction was then concentrated and the residue taken up in a mixture of methylene chloride (500 mL) and 2N NaOH (500 mL). The organic phase was separated and the aqueous phase extracted with additional methylene chloride (300 mL). The organic phases were dried and concentrated, and the residue chromatographed on SiO₂ (0-13% NH₃/MeOH in CH₂Cl₂) to produce a yellow solid (9.26 g). The mixed fractions from the column were rechromatographed to provide an additional 3.23 g of the desired material. Total yield 12.49 g (65% yield over the last two steps).

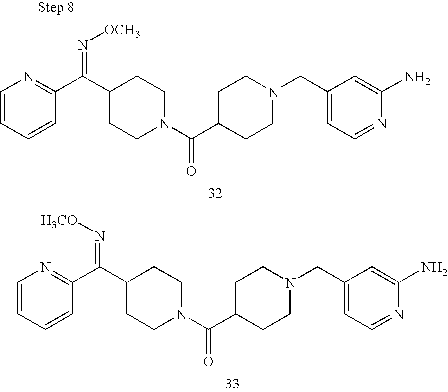

Compound 31 (1 g) in ethanol (15 mL) was separated into the pure isomers using a Chiralcel AD column (20 mm×500 mm) (eluent: 75:25 hexane:isopropanol plus 0.5% N,N-diethylamine; flow rate: 50 mL/min; UV detection at 254 nM) to provide compound 32 (0.6 g) and compound 33 (0.4 g). [M+H]⁺ 437 for 32 and 33.

### Example 7

### Synthesis of Compound 41

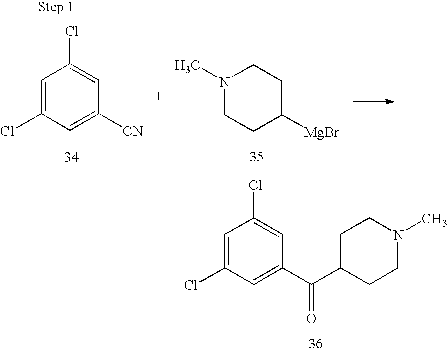

To a solution of 34 (2.4 g, 13.5 mmol) in THF (15 mL) was added compound 35 (26 mL of a 1.3M solution) and the reaction stirred overnight at room temperature. 2N HCl was then added till the pH<2 and the THF was removed under reduced pressure. The pH was neutralized by the addition of 1N NaOH and the aqueous phase extracted with 5% MeOH in EtOAc. The organic phase was dried, concentrated, and the residue chromatographed (20% MeOH in EtOAc) to provide 36 (1.03 g, 28%).

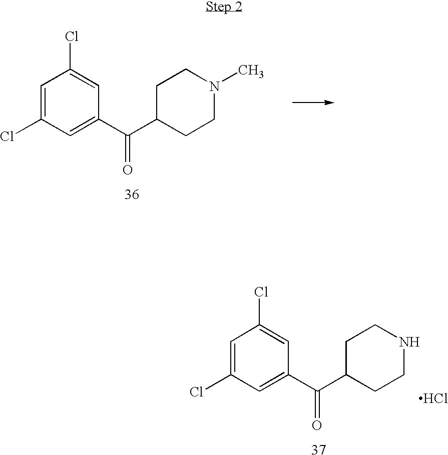

To a solution of 36 (1.03 g, 3.78 mmol) in 1,2-dichloroethane (30 mL) was added 1-chloroethylchloro formate (0.76 mL, 7.6 mmol) and the reaction stirred at room temperature overnight. The solvent was removed in vacuo and the residue washed with ether. Solid residue was removed by filtration and the ether removed by evaporation to provide an oil which was dissolved in MeOH (15 mL) and heated to reflux for 2 h. Removal of the solvent gave 37 which was used in the next step without further purification (1.4 g).

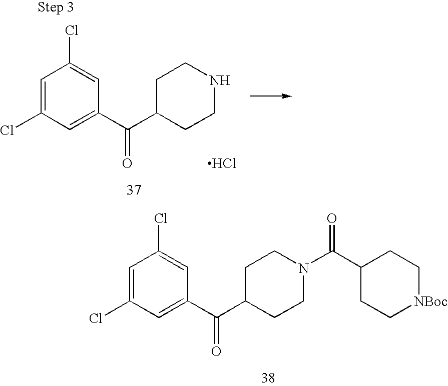

Compound 37 (0.98 g, 3.78 mmol), N-Boc isonipocotic acid (0.87 g, 3.78 mmol), DEC (1.11 g, 5.7 mmol), HOBT (0.68 g, 4.91 mmol) and DIPEA (3 mL) were combined in CH₂Cl₂ (40 mL) and stirred overnight at room temperature. The reaction was then diluted with CH₂Cl₂ and washed with saturated aqueous NaHCO₃. The organic layer was dried, concentrated and the residue chromatographed (10% hexane in EtOAc) to provide 38 (1.61 g, 91%).

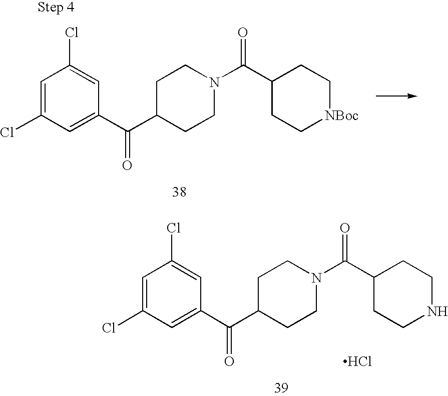

Compound 38 (1.61 g, 3.43 mmol) in CH₂Cl₂ (15 mL) was treated with 1N HCl in dioxane (5.2 mL) and stirred overnight at room temperature. The solvent was removed in vacuo to provide 39 (1.65 g) which was used without further purification.

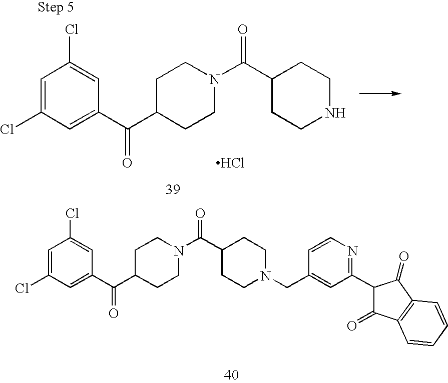

Compound 39 (1.65 g, 4.01 mmol), 7 (1.29 g, 4.07 mmol) and Et₃N (1.7 mL) were combined in DMF (40 mL) and stirred at room temperature overnight. The reaction was dissolved in EtOAc and washed 4 times with water. The organic layer was dried and concentrated, and the residue purified by chromatography (5% MeOH in EtOAc) to provide 40 (0.6 g, 47%).

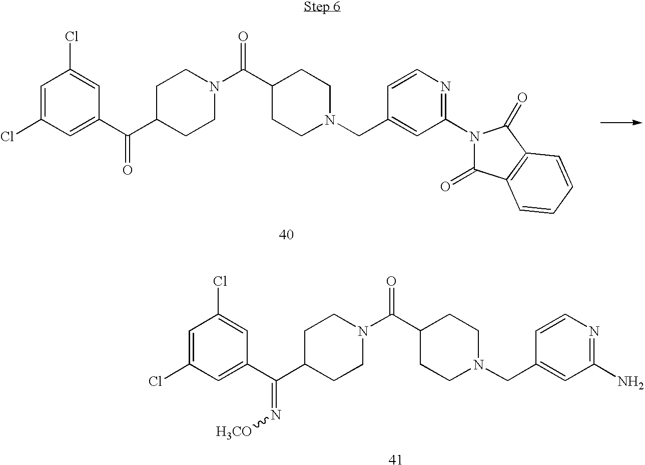

A solution of 40 (0.31 g, 0.51 mmol) in pyridine (5 mL) was treated with H₂NOMe.HCl (0.092 g, 1.08 mmol) and heated to 60° C. overnight. The reaction was diluted with 10% MeOH in CH₂Cl₂, washed with saturated aqueous NaHCO₃, dried, and concentrated, and the residue purified by chromatography (10-15% MeOH in EtOAc) to provide 41 (0.09 g).

### Example 8

### Synthesis of Compound 45

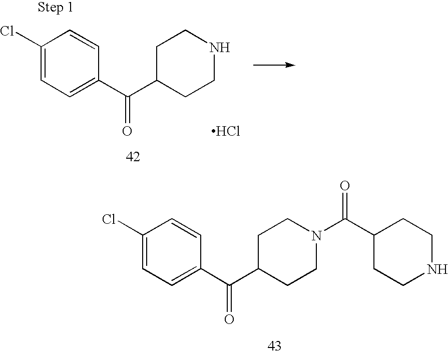

In a manner similar to that described in Example 7, Steps 3-4, compound 42 was converted to compound 43.

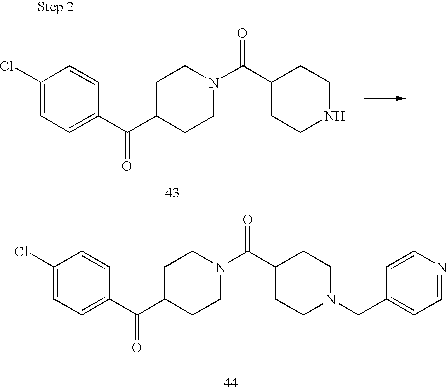

To a solution of 43 (2.3 g, 6.3 mmol) in CH₂Cl₂ (60 mL) was added 4 Å molecular sieves and 4-formylpyridine (0.68 mL, 6.9 mmol) and the mixture stirred for 3 h at room temperature. Na(OAc)₃BH (2.7 g, 12.7 mmol) was then added and the reaction stirred for 1 h. The reaction was quenched by the addition of NH₄Cl followed by the addition of saturated aqueous NaHCO₃. The reaction mixture was then extracted with EtOAc, and the combined organic layers were dried and concentrated to provide a residue which was chromatographed (20% MeOH in EtOAc). Compound 44 was obtained (2.3 g, 87%).

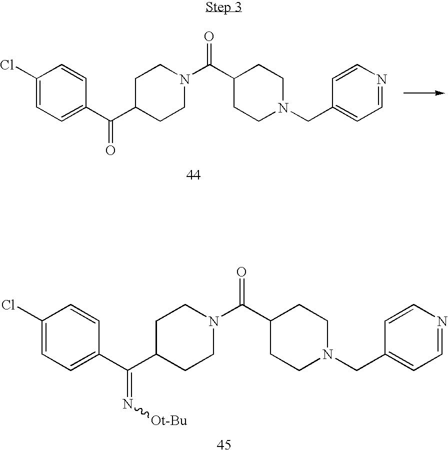

In a manner similar to that described in Example 7, Step 6, compound 44 was converted to compound 45.

### Example 9

### Synthesis of Compound 50

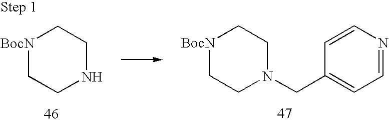

In a manner similar to that described in Example 8, Step 2, compound 46 (1.13 g, 6 mmol) was converted to compound 47 (1.7 g, 100%).

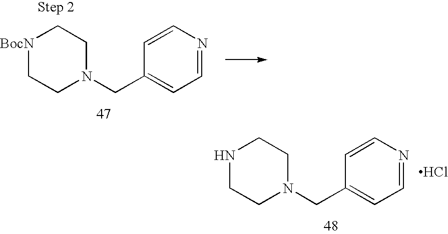

In a manner similar to that described in Example 7, Step 4, compound 47 (1.7 g, 6.13 mmol) was converted to compound 48 (1.9 g, 100%).

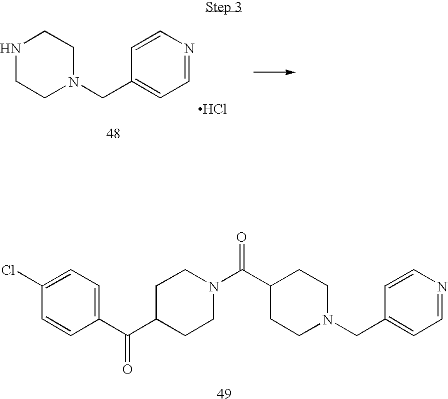

A mixture of compound 48 (0.57 g, 2 mmol) and compound 42 (0.52 g, 2 mmol) in CH₂Cl (20 mL) was added Et₃N (1.95 mL) and the reaction cooled to −40° C. Triphosgene (0.2 g) was added and the reaction stirred at −40° C. for 2 h and room temperature for 48 h. The reaction was then washed with 1N NaOH, brine, and the organic layer dried. Concentration gave a residue that was purified by column chromatography (10% MeOH in EtOAc) to provide 49 (0.14 g, 55%).

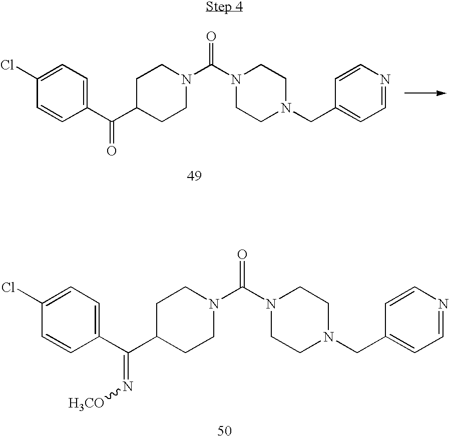

In a manner similar to that described in Example 7, Step 6, compound 49 (0.09 g, 0.21 mmol) was converted to compound 50.

### Example 10

### Synthesis of Compounds 54, 55, 56 and 57A

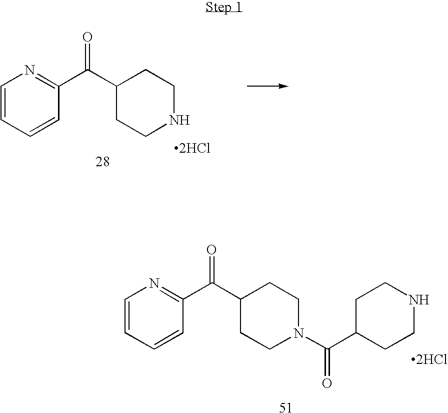

In a manner similar to that described in Example 7, Steps 3-4, compound 28 (2.6 g, 9.9 mmol) was converted to compound 51 (1.1 g).

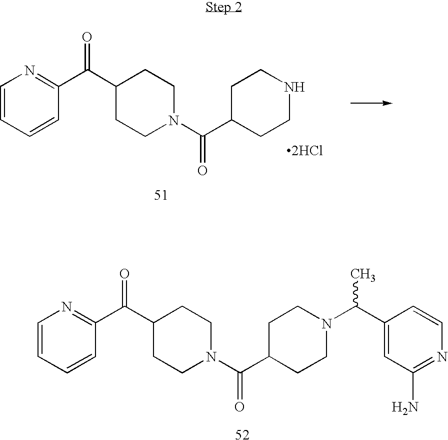

In a manner similar to that described in Example 7, Step 5, compound 51 (1.1 g, 2.94 mmol) was reacted with compound 11 (0.59 g, 2.94 mmol) to provide compound 52 (0.53 g).

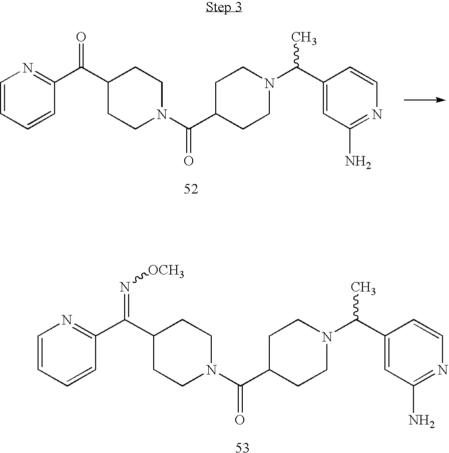

In a manner similar to that described in Example 6, Step 7, compound 52 (0.53 g, 1.26 mmol) was converted to compound 53 (0.48 g).

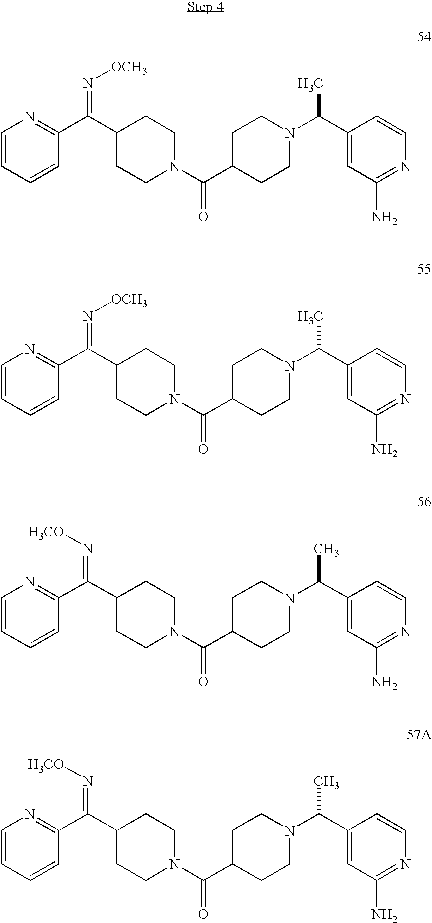

In a manner similar to that described in Example 6, Step 8, the 4 diastereomers of compound 53 could be obtained using a Chiralcel AD column (75:25 hexane:EtOAc plus 0.5% Et₂NH). The two faster eluting compounds (54 and 55) were the E-oxime isomers and the slower eluting compounds (56 and 57A) were the Z-oxime isomers.

| | | |
|---|---|---|
| Isomer A | 54 | 0.12 g |
| Isomer B | 55 | 0.11 g |
| Isomer C | 56 | 0.08 g |
| Isomer D | 57A | 0.06 g |

### Example 11

### Synthesis of Compound 59

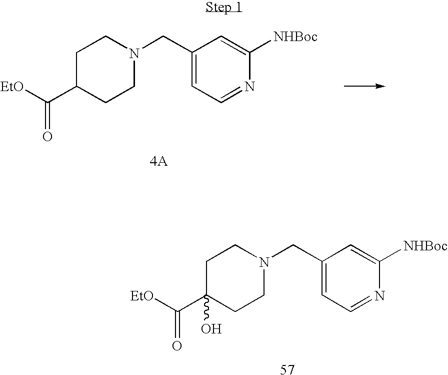

A solution of n-BuLi (4.2 mL of a 1.6 M solution in hexane) in THF (25 mL) was treated at −25° C. with (i-Pr)₂NH (0.69 g, 6.8 mmol). The reaction was stirred for 1 h at 0° C. and then cooled to −70° C. Compound 4A (0.82 g, 2.26 mmol) in THF (5 mL) was added dropwise and the reaction stirred at −70° C. for 2 h and −50° C. for 2 h. The reaction was recooled to −70° C. and (1S)-(+)-(10-camphorsulfonyl)oxaziridine (1.04 g, 4.52 mmol) in THF (5 mL) was added. The reaction was stirred at −70° C. for 2 h and slowly warmed to room temperature overnight. The reaction was quenched by the addition of saturated aqueous NH₄Cl and extracted with EtOAc. The organic layer was dried and concentrated, and the residue purified by column chromatography (1:1 hexane:EtOAc) to provide 57 (0.44 g, 51%).

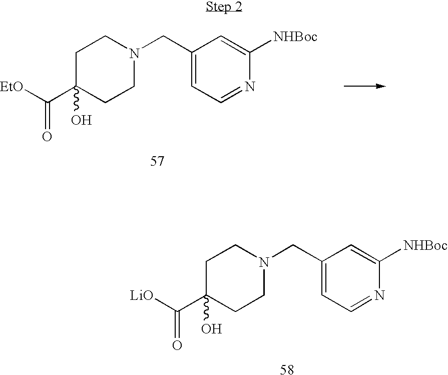

In a manner similar to that described in Example 1, Step 5, compound 57 (0.42 g, 1.1 mmol) was converted to compound 58 (0.4 g).

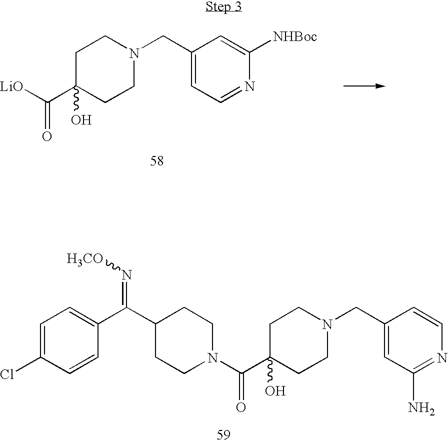

In a manner similar to that described in Example 6, Steps 5-8, compound 58 (0.25 g, 0.7 mmol) was converted to compound 59 (0.1 g).

### Example 12

### Synthesis of Compound 65

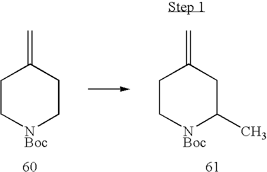

A solution of compound 60 (10 g, 50.7 mmol) in ether (150 mL) at −78° C. was treated sequentially with TMEDA (11.8 g, 101.4 mmol) and s-BuLi (58.5 mL of a 1.3M solution in hexanes, 76 mmol) and the reaction stirred at this temperature for 6 h. Neat CH₃SO₄CH₃ (12.8 g, 101.4 mmol) was then added and the reaction allowed to slowly warm to room temperature overnight. Saturated aqueous NaCl was added and the organic layer was separated. The aqueous layer was extracted three times with ether and the combined organic layers were dried, concentrated, and the residue chromatographed (5% EtOAc in hexane) to provide 61 (8.0 g, 75%).

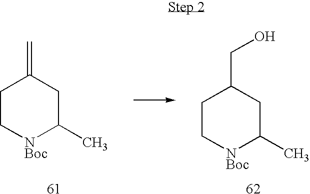

A solution of 61 (8 g, 37.9 mmol) in THF (40 mL) at 0° C. was treated dropwise with a solution of BH₃.THF (45.4 mL of a 1.0M solution in THF, 45.4 mmol) and the reaction allowed to slowly warm to room temperature overnight. The reaction was recooled to 0° C., EtOH (13 mL), pH=7 buffer (25 mL) and H₂O₂ (25 mL) was added, and the reaction allowed to stir at room temperature overnight. The solvent was then removed in vacuo and the residue poured into water and CH₂Cl₂. 10% aqueous NaOH (10 mL) was added and the organic layer separated. The aqueous layer was extracted with additional CH₂Cl₂ and the combined organic layers were dried and concentrated. The residue was chromatographed (40% EtOAc in hexane) to provide 62 (3 g).

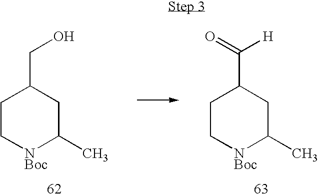

A solution of 62 (2.8 g, 12.2 mmol) in EtOAc (30 mL) and NaBr (1.26 g, 0.12 mmol) in saturated aqueous NaHCO₃ (30 mL) was cooled to 0° C. and treated with TEMPO (0.02 g, 0.12 mmol). After 15 min., NaOCl (17.44 mL) was added and the mixture stirred for 3 h. Saturated aqueous Na₂S₂O₃ was added and the pH adjusted to 5-6 by the addition of 1N HCl. The mixture was extracted with EtOAc and the organic layers were dried and concentrated. The residue was chromatographed (10-20% EtOAc in hexane) to provide compound 63 (2.1 g, 76%).

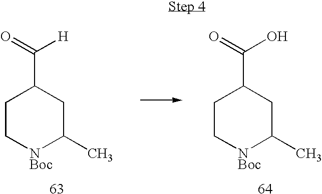

To a cooled (0° C.) suspension of PCC (0.95 g, 4.4 mmol) in CH₂Cl₂ (5 mL) was added dropwise a solution of 63 (0.5 g, 2.2 mmol). And the mixture stirred overnight at room temperature. Additional PCC (1 eq.) was added and the mixture was heated to reflux for 2 h. The reaction was cooled, filtered through celite, and concentrated to provide crude 64 (1.5 g) which was used without further purification.

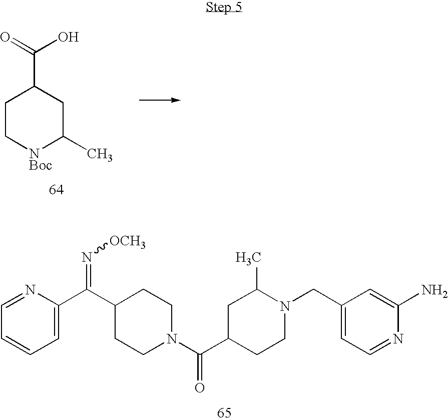

In a manner similar to that described in Example 5, Step 5, Example 7, Step 4, Example 1, Step 4, and Example 6, Steps 6 and 7, 64 (0.73 g, 3 mmol) was converted to 65 (0.1 g).

### Example 13

### Synthesis of Compound 70

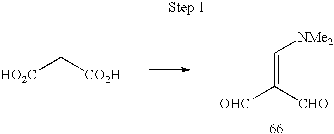

Dialdehyde 66 was prepared from malonic acid and POCl₃-DMF as described in Collect. Czech. Chem. Comm. 1961, 26, 3051.

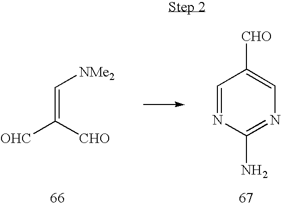

To a mixture of 900 mg (7.1 mmol) of dialdehyde 66 and 678 mg (7.1 mmol) of guanidine hydrochloride in 20 mL of absolute ethanol was added 483 mg (7.1 mmol) of sodium ethoxide. Reaction mixture was heated at 90° C. for 12 h, cooled to room temperature, concentrated—dry loaded on silica gel and flash chromatographed (0-10% MeOH/20-30% acetone/CH₂Cl₂) to produce 355 mg (2.9 mmol; 41%) of 67 as a yellowish solid.

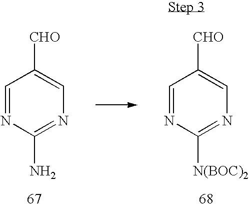

To a mixture of 166 mg (1.35 mmol) of aminopyrimidine 67, 17 mg (0.14 mmol) of DMAP and 418 μL (3.00 mmol) of Et₃N in 10 mL of THF was added 589 mg (2.7 mmol) of (BOC)₂O. The mixture was stirred at room temperature for 5 h, concentrated-dry loaded on silica gel and flash chromatographed (1-3% acetone/CH₂Cl₂) to produce 117 mg (0.36 mmol; 27%) of 68 as a clear oil.

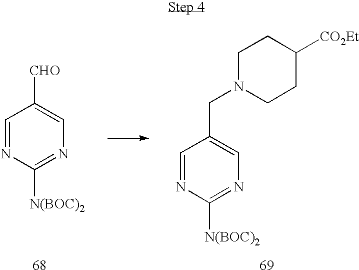

To a solution of 117 mg (0.36 mmol) of aldehyde 68 in 7 mL of CH₂Cl₂ was added 67 (0.43 mmol) of ethyl isonipecotate and 5 μL of acetic acid. 30 min. later 153 mg (0.72 mmol) of NaBH(OAc)₃ was introduced. The mixture was stirred overnight at room temperature, diluted with CH₂Cl₂, washed with aqueous NaHCO₃, dried and concentrated, and crude residue was flash chromatographed (0-4% sat. NH₃ in MeOH/CH₂Cl₂) to produce 133 mg (0.29 mmol; 81%) of 69 as a white film.

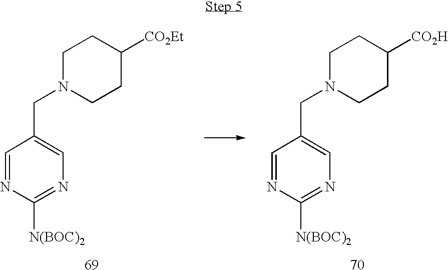

To a solution of ester 69 in 5 mL of a 3:1:1 mixture of THF-water-methanol was added 11 mg (0.44 mmol) of LiOH. Reaction mixture was stirred overnight at room temperature, concentrated to dryness and exposed to high vacuum to obtain 134 mg of crude acid 70 as a yellowish solid which was used without purification.

### Example 14

### Synthesis of Compound 74

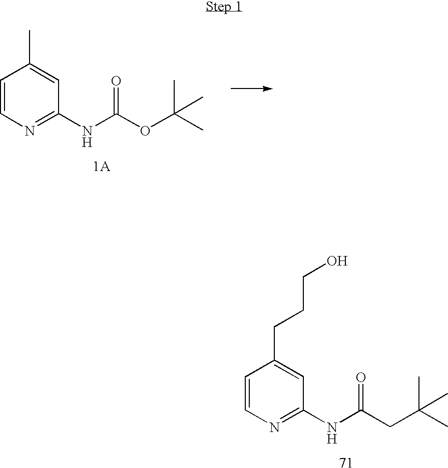

To a −78° C. solution of 2.36 g (11.4 mmol) of picoline 1A in 70 mL of THF was added 16.3 mL of 1.4 M BuLi solution (22.8 mmol) in hexanes in portions over 10 min. Reaction mixture was then allowed to warm up and was then stirred for 2 h at room temperature, which resulted in the formation of an orange precipiate. The mixture was cooled back to −78° C., and ethylene oxide was bubbled through the solution for 1 min. followed by stirring for 5 min. This two-step sequence was repeated eight times. The mixture was then allowed to warm to −50° C., stirred at that temperature for 40 min., quenched with 1.34 mL (23 mmol) of AcOH and allowed to warm to room temperature. Dilution with water was followed by extraction with EtOAc, concentration of the organic phase, and flash chromatography of the crude residue (10-15% acetone/CH₂Cl₂) to produce 1.50 g (5.95 mmol; 53%) of 71 as a white solid.

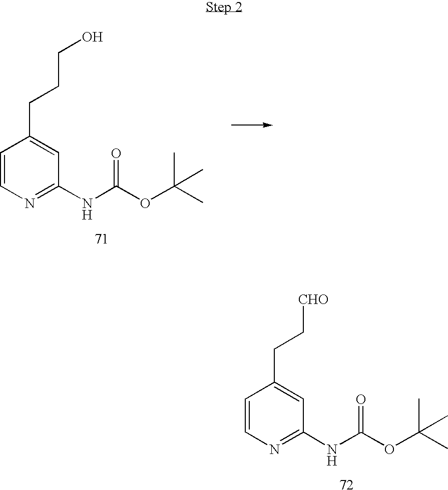

To a −60° C. solution of 628 μL (7.2 mmol) of oxalyl chloride in 20 mL of CH₂Cl₂ was added dropwise 1.03 mL (14.5 mmol) of DMSO. After stirring the mixture for 15 min. at −55° C., a solution of 1.50 g (5.95 mmol) of alcohol 71 in 20 mL of CH₂Cl₂ was introduced over the period of 15 min. After the addition was complete, the mixture was stirred for 30 min. at −55° C., followed by the addition of 4.18 mL (30.0 mmol) of Et₃N and stirring for another 15 min. The reaction mixture was then warmed to room temperature and diluted with water. Extraction with CH₂Cl₂ was followed by concentration of the organic phase and flash chromatography (1-15% acetone/CH₂Cl₂) to produce 1.00 g (4.00 mmol; 67%) of 72 as an off-white solid.

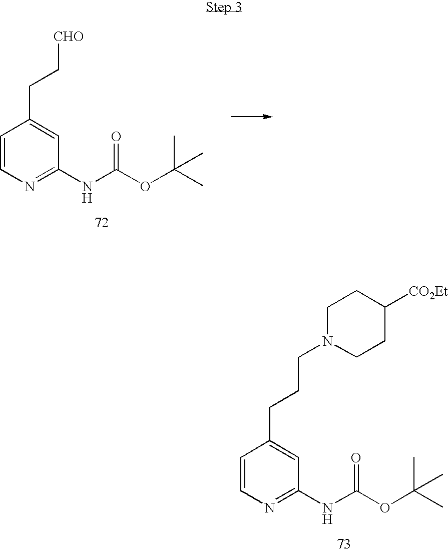

To a solution of 1.00 g (4.0 mmol) of aldehyde 72 in 25 mL of CH₂Cl₂ was added 617 μL (4.8 mmol) of ethyl isonipecotate followed by one drop of AcOH. Reaction mixture was then stirred for 40 min at room temperature after which 1.70 g (8.0 mmol) of NaBH(OAc)₃ was introduced. Reaction mixture was stirred overnight at room temperature, neutralized with saturated aqueous NaHCO₃, diluted with water and extracted with CH₂Cl₂. Concentration and flash chromatography (0-4% saturated NH₃ in MeOH/CH₂Cl₂) provided 1.41 g (3.6 mmol; 90%) of 73 as a white solid.

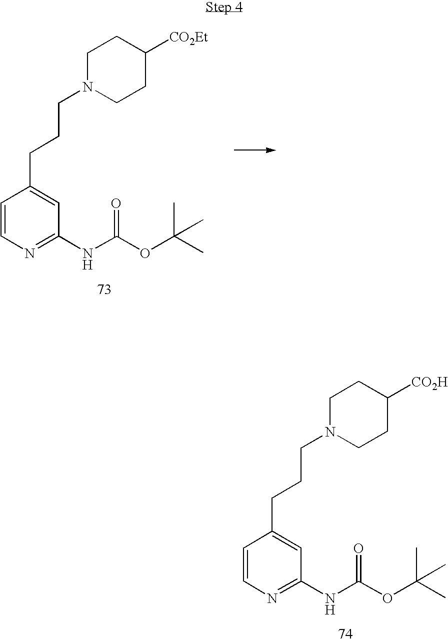

To a solution of 534 mg (1.47 mmol) of ester 73 in 4 mL of a 3:1:1 mixture of THF-water-methanol was added 60 mg (2.50 mmol) of LiOH. Reaction mixture was stirred overnight at room temperature, concentrated to dryness and exposed to high vacuum to obtain 540 mg of crude acid 74 as a white solid which was used without purification.

### Example 15

### Synthesis of Compound 75

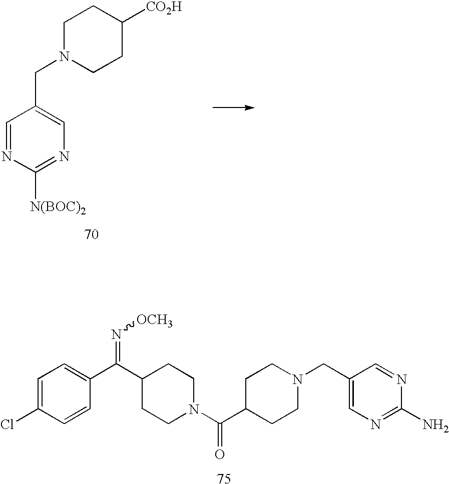

In a manner similar to that described in Example 6, steps 5, 6, and 7, 70 was converted to 75.

### Example 16

### Synthesis of Compound 76

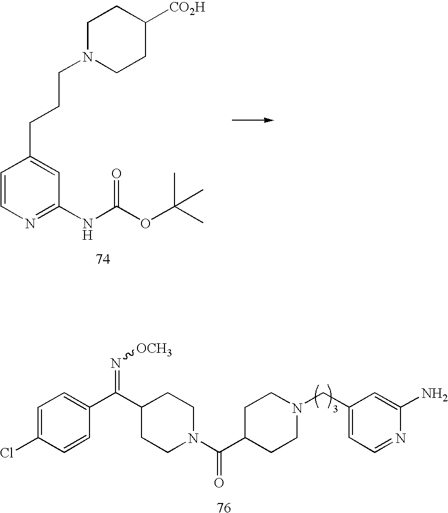

In a manner similar to that described in Example 6, steps 5, 6, and 7, compound 74 was converted to 76.

### Example 17

### Synthesis of Compound 80

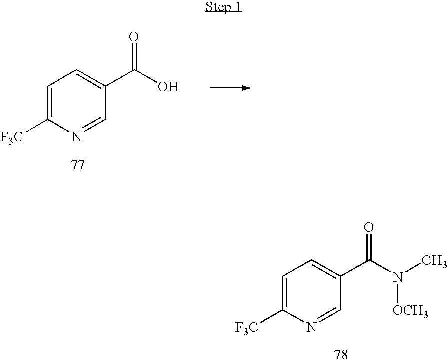

To a solution of 77 (0.73 g, 3.82 mmol) in CH₂Cl₂ (10 mL) was added (COCl)₂ (0.41 mL, 4.58 mmol) followed by DMF (0.1 mL) and the reaction was maintained at 40° C. for 3 h. The reaction was then concentrated to provide a brown solid which was dissolved in CH₂Cl₂ (10 mL). N,O-dimethyl-OHamine hydrochloride (0.56 g, 5.73 mmol) and DIPEA (1.33 mL) were added and the reaction was stirred at room temperature overnight. The reaction was quenched by the addition of saturated aqueous NaHCO₃ and extracted with EtOAc. The combined organic layers were dried and concentrated, and the residue purified by chromatography to provide 78 (3.2 g, 84%).

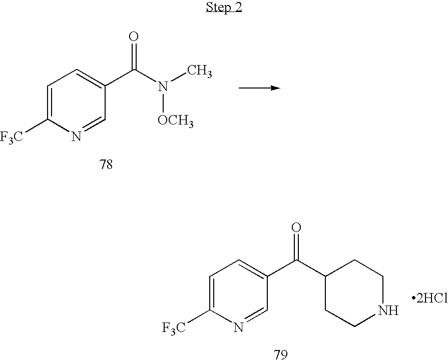

In a manner similar to that described in Example 5, steps 1 and 4, 78 (0.57 g, 2.41 mmol) was converted to 79 (0.59 g).

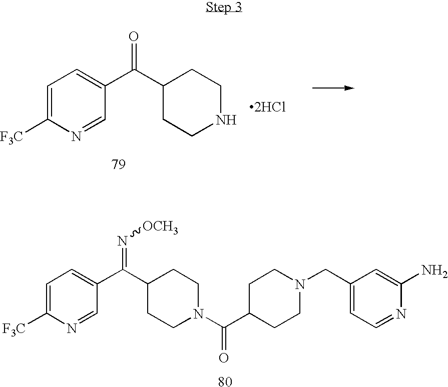

In a manner similar to that described in Example 6, steps 5, 6 and 7, 79 (0.38 g, 1.49 mmol) was converted to 80 (0.24 g).

### Example 18

### Synthesis of Compound 83

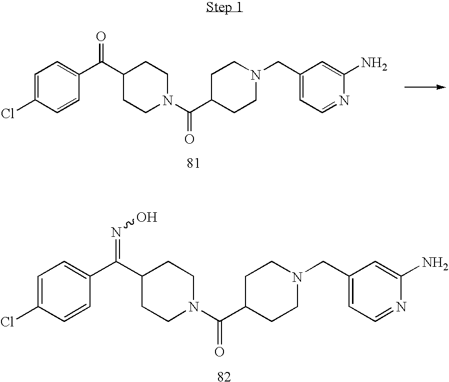

In a manner similar to that described in Example 6, step 7, 81 (0.36 g, 0.53 mmol; synthesized in the same manner as compound 30) was converted to 82 (0.34 g, 63%).

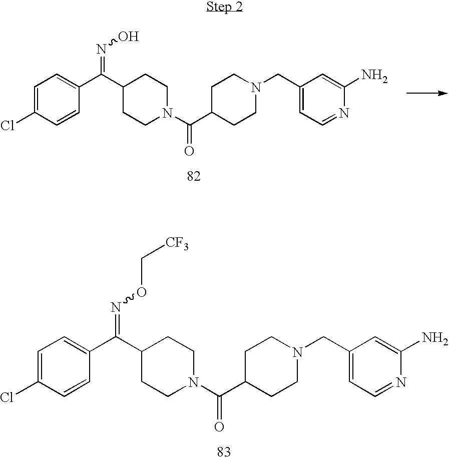

To a solution of 82 (0.115 g, 0.25 mmol) in DMF (4 mL) was added NaH (60% dispersion in mineral oil, 0.03 g, 0.76 mmol). After 5 h at room temperature, CF₃CH₂OSO₂CF₃ (0.069 g, 0.3 mmol) was added and the reaction stirred at room temperature overnight. The reaction was diluted with EtOAc and extracted 3 times with water to remove the DMF. The organic layer was dried and concentrated to provide a residue which was purified by chromatography (10% MeOH/NH₃ in EtOAc) to provide 83 (0.08 g, 30%).

### Example 19

### Synthesis of Compound 88

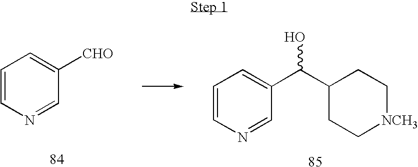

To a solution of 17 (0.21 mole, 100 ml THF, −10° C.) was added 84 (0.14 mole) over 5 min and the reaction mixture became very viscous. Additional THF (100 ml) was added and the yellow suspension was warmed from −10° C. to 10° C. over about 2.5 hr. The reaction was quenched by the addition of 100 ml saturated NH₄Cl and 100 ml H₂O. Extracted once with EtOAc (300 ml) and eight times with CH₂Cl₂ (150 ml). Dried over solid MgSO₄ and filtered. Concentrated and flashed over silica gel chromatography (3 to 10% MeOH (NH₃)/CH₂Cl₂) to obtain 85 (11 g, yield: 38%).

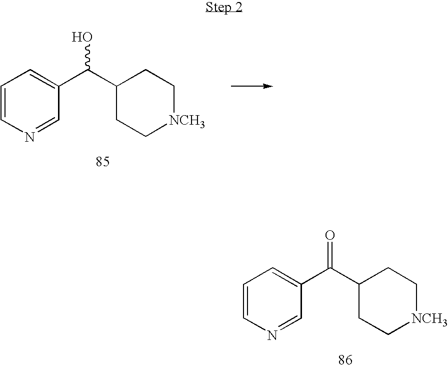

To the mixture of 85 (9.2 g) and MnO₂ (42 g) was added 200 ml CH₂Cl₂, and the mixture was stirred at room temperature overnight. Additional MnO₂ (20 g) was added and the reaction was stirred another 24 hrs. The MnO₂ was filtered off and the reaction was concentrated and flashed over silica gel (5% and 10% MeOH (NH₃)/CH₂Cl₂) to provide 86 (3.1 g, yield: 33%).

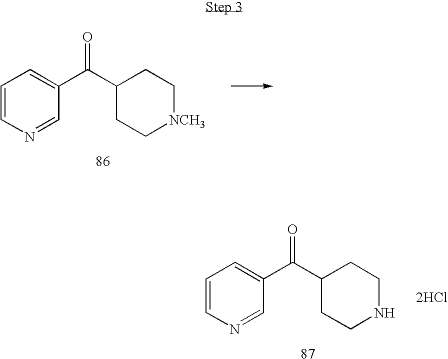

In a manner similar to that described in Example 7, step 2, 86 (3.1 g) was converted to 87 (2.0 g, yield: 68%).

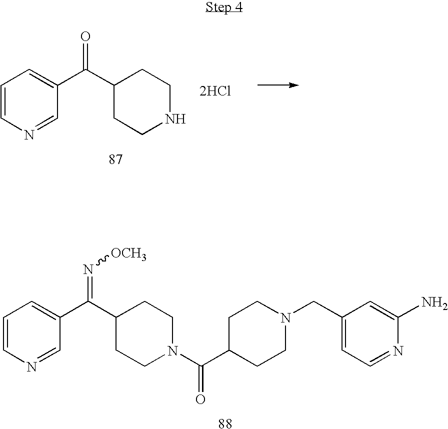

In a manner similar to that described in Example 7, step 3, 4, and 6, 87 was converted to 88.

### Example 20

### Synthesis of Compound 92

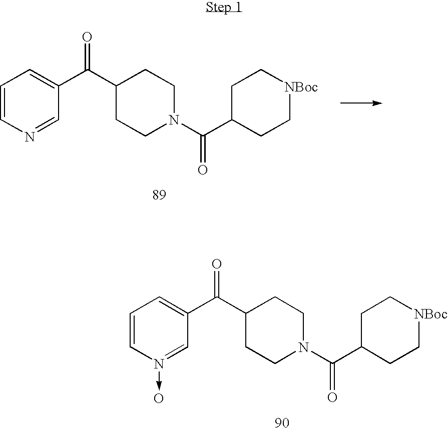

To the solution of compound 89 in CH₂Cl₂ (20 ml) at 0° C. was added m-CPBA (0.54 g) and the reaction was stirred at 0° C. for 25 min. and then at room temperature stirred for 2 hrs. 40% NH₄OH (12 ml) was added and the mixture was stirred for 30 min. Separated and extracted the aqueous layer with CH₂Cl₂ (10 ml). Dried (MgSO₄), filtered and concentrated in vacuo. Flash chromatography (5% MeOH(NH₃)/CH₂Cl₂) gave 90 (0.67 g, 80%).

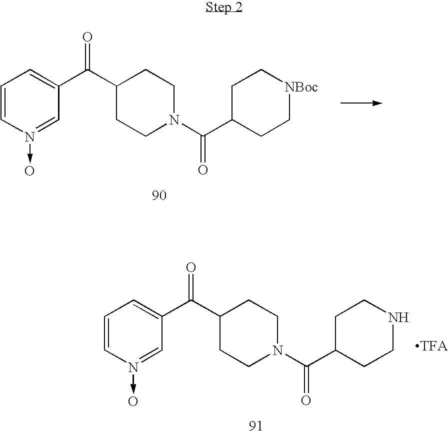

To the solution of 90 (0.65 g) in CH₂Cl₂ (6 ml) at −10° C. was added TFA (6 ml) and the reaction was stirred for 1 hr from −10° C. to 0° C. Concentrated down and azeotroped twice with toluene (20 ml), and concentrated to dryness to obtain 91 as a gummy oil which was used as is.

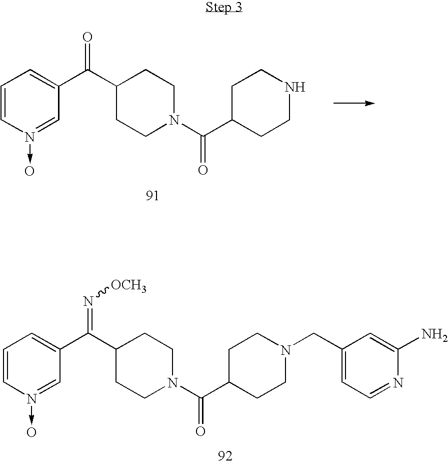

In a manner similar to that described in Example 7, steps 5 and 6, 91 was converted to 92.

### Example 21

### Synthesis of Compound 99

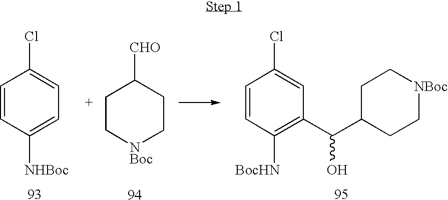

To a solution of 93 (5.17 g, 22.7 mmol) in THF (100 mL) at −50° C. was added s-BuLI (38.4 mL of a 1.3M solution in hexane, 49.9 mmol) dropwise. After 1.5 h at −40° C., the reaction was recooled to −50° C. and 95 (4.84 g, 22.7 mmol) in THF (20 mL) was added. After 2.75 h at −50° C., glacial acetic acid was added followed by saturated aqueous NH₄Cl. The mixture was warmed to room temperature and the layers were separated. The aqueous layer was extracted with EtOAc. The combined organic layers were dried (MgSO₄) filtered and concentrated to provide a residue that was purified by flash column chromatography (1% to 3% MeOH/NH₃ in CH₂Cl₂) to provide 95 (6.35 g, 63%).

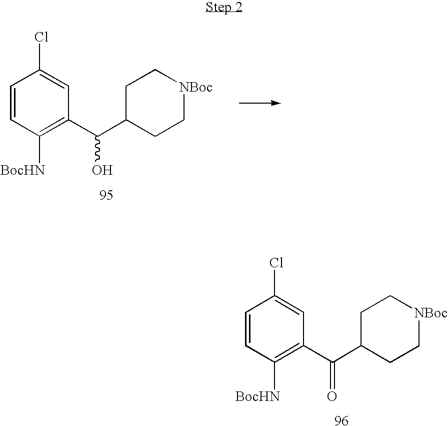

In a manner similar to that described in Example 12, step 3, 95 (5.34 g, 12.11 mmol) was converted to 96 (4.71 g, 75%).

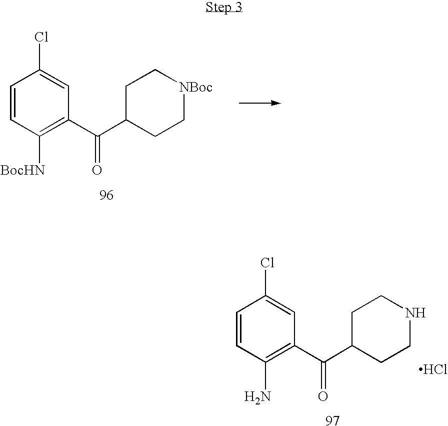

In a manner similar to that described in Example 6, step 4, 96 (3.7 g, 8.43 mmol) was converted to 97 (3.08 g, >100%) which was used as is in the next step.

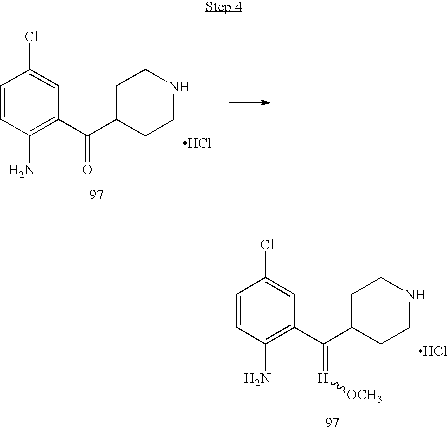

Compound 97 (0.7 g, 2.25 mmol), H₂NOCH₃.HCl (0.94 g, 11.23 mmol) and NaOAc (1.47 g, 17.97 mmol) were combined in 1-pentanol (20 mL) and water (2 mL) and heated to reflux for 2 days. The reaction was cooled to room temperature and 0.5N NaOH was added. The EtOH was removed in vacuo, additional water (15 mL) was added, and the reaction extracted with 10% EtOH in CH₂Cl₂ (180 ML total volume). The combined organic extracts were dried and concentrated to provide 98 (0.55 g, 92%).

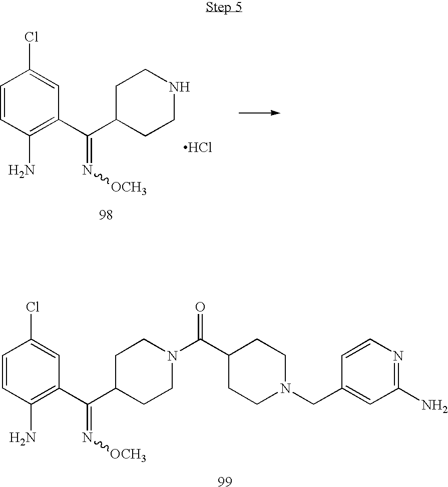

In a manner similar to that described in Example 6, steps 5, 6, and 7, 98 was converted to 99.

### Example 22

### Synthesis of Compound 104

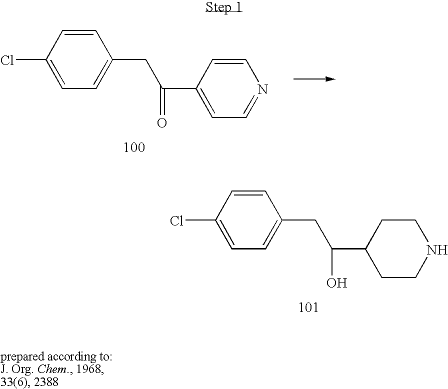

A solution of 2.2 g (9.5 mmol) of 100 in 75 mL of glacial acetic acid was hydrogenated in the presence of 0.5 g of 10% w/w platinum-on-charcoal for 5 h. The reaction mixture was filtered to remove the catalyst and the filtrate was concentrated by evaporation under reduced pressure to produce a solid residue which was basified with 0.5N NaOH and extracted with methylene chloride (CH₂Cl₂). Methylene chloride extracts were dried over anhydrous MgSO₄ and concentrated. The residue was purified by flash chromatography eluted with 10-30% of 7N NH₃-MeOH in CH₂Cl₂ to provide 0.82 g of 101 (mp 158-163° C.). LCMS m/z 240 (M+H).

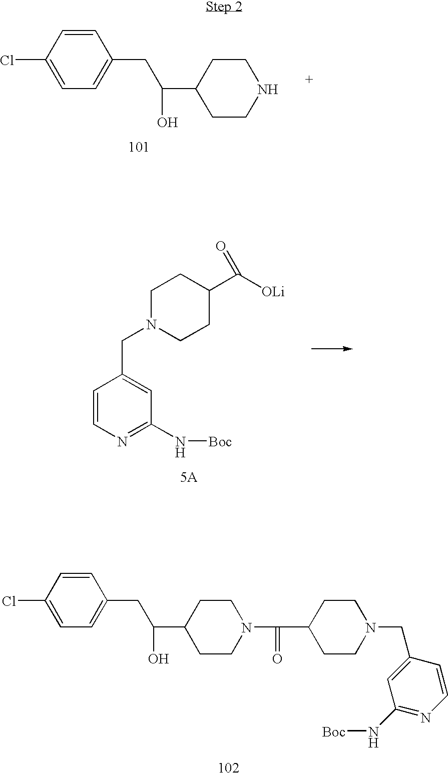

A mixture of 0.12 g (0.52 mmol) of 101, 0.2 g (0.52 mmol) of 5A, 0.67 g (0.5 mmol) of 1-hydroxybenzotriazole hydrate (HOBt), and 0.11 g (0.57 mmol) of 1-(3-dimethylaminopropyl)-3-ethylcarbodiimide hydrochloride (DEC) in 7 mL of anhydrous dimethylformamide (DMF) was stirred at ambient temperature for 18 h. The mixture was diluted with water and the resulting precipitate was filtered to produce 0.26 g of 102 as a white solid (mp 110-115° C.). LCMS m/z 557 (M+H).

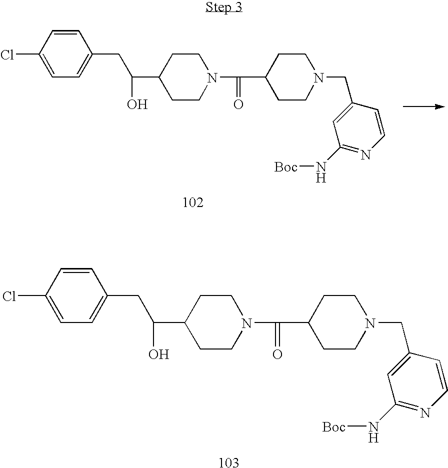

To a stirred solution of 0.34 g (2.7 mmol) of oxalyl chloride in 3 mL of anhyrous CH₂Cl₂ at −70° C. was added 0.44 g (5.7 mmol) of anhyrous methylsulfoxide in 2 mL of CH₂Cl₂. After being stirred at −70° C. for 10 minutes, the reaction mixture was added 1.2 g (2.15 mmol) of 102 in 10 mL of CH₂Cl₂. The stirred mixture was kept at −70° C. for 0.5 h, mixed with 1.8 mL (13 mmol) of triethylamine, and then allowed to warm up to ambient temperature by itself. The mixture was diluted with water and extracted with CH₂Cl₂. Organic extracts were washed with brine, dried over anhydrous MgSO₄ and concentrated to produce 1.18 g of 103 as a glass. LCMS 555 (M+H).

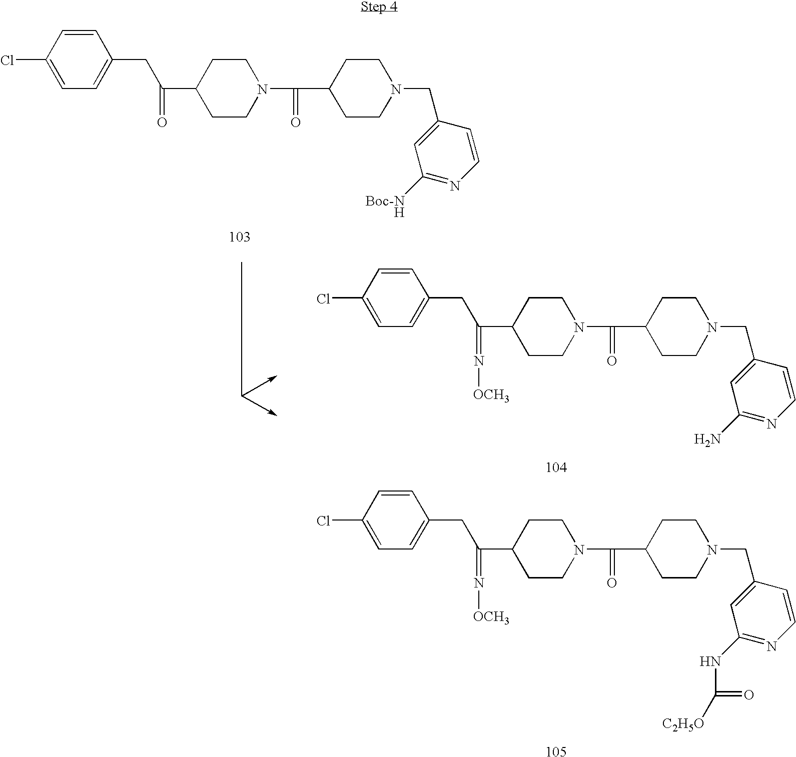

A solution of 0.8 g (1.44 mmol) of 103 and 0.6 g (7.2 mmol) of methoxylamine hydrochloride in 40 mL of ethanol and 40 mL of pyridine was heated under reflux for 18 h. The mixture was concentrated and the residue was taken up in ethyl acetate/ether and washed with water. The organic solution was dried over anhydrous MgSO₄ and concentrated to 0.65 g of viscous residue which was dissolved in 8 mL of trifluoroacetic acid and 8 mL of CH₂Cl₂ and stirred at ambient temperature for 18 h. The solution was concentrated and the residue was basified with 1N NaHCO₃ and extracted with ethyl acetate. Organic extracts were washed with brine, dried over anhydrous MgSO₄ and concentrated to a gummy residue. Purification of this residue by flash chromatography with 5-8% of 7N NH₃-MeOH in CH₂Cl₂ produced 0.151 g of 104 as a gum, LCMS m/z 484 (M+H) and 0.146 g of 105 as a glass, LCMS m/z 556 (mH+).

Mixing a solution of 0.056 g of the free base of 104 in ethyl acetate with a solution of 0.04 g of maleic acid in ethyl acetate produced a precipitate which was isolated by filtration to provide 0.06 g of a dimaleate salt of 104 (mp 155-160° C.).

### Example 23

### Synthesis of Compound 111

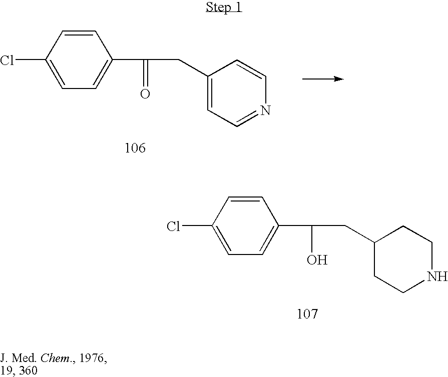

2.4 g (10. mmol) of 106 were reduced in the similar manner as that described in Example 22, step 1 to provide 1.5 g of 107 as a semi-solid. LCMS m/z 240 (M+H).

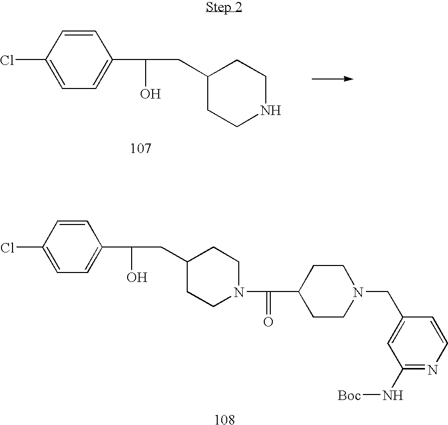

1.5 g (6.31 mmol) of 107 were coupled with 3 in the similar manner as that described in Example 22, step 2 to provide 3 g of 108 as a solid (mp 104-106° C.). LCMS m/z 557 (M+H).

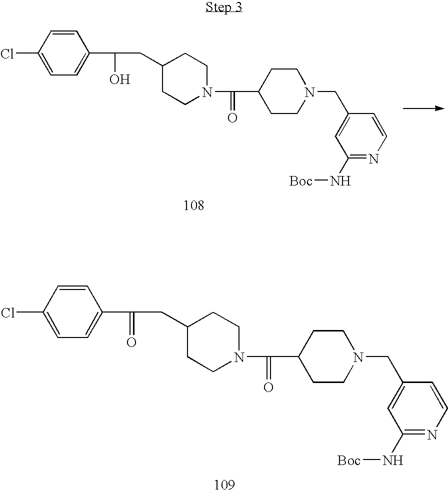

1.17 g (2.1 mmol) of 108 were oxidized in the similar manner as that described in Example 22, step 3 to provide 0.7 g of 109 as a glass. LCMS m/z 557 (M+H).

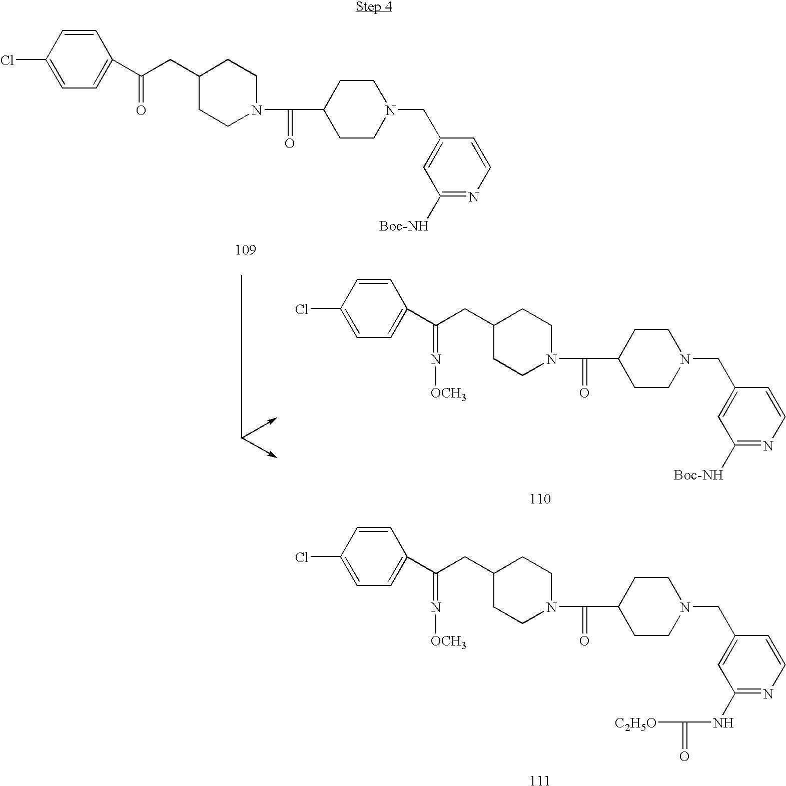

0.32 g (0.58 mmol) of 109 were reacted with 0.6 g (7.2 mmol) of methoxylamine hydrochloride in the same manner as that described in Example 22, step 4 to provide 0.065 g of 110 as a gum, LCMS m/z 484 (M+H) and 0.12 g of 111 as a glass, LCMS m/z 556 (M+H).

### Example 24

### Synthesis of Compound 117

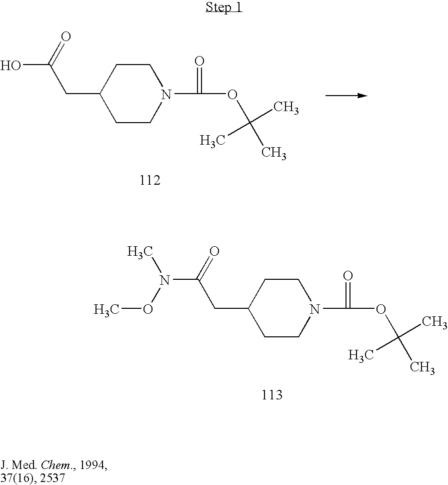

A mixture of 18 g (74 mmol) of 112, 7.2 g (74 mmol) of N,O-dimethylhydroxylamine hydrochloride, 19.4 g (15 mmol) of N,N-diisopropylethylamine, 1.1 g (8 mmol) of HOBt and 14.2 g (74 mmol) of DEC in 80 mL of anhydrous DMF was stirred at ambient temperature for 18 h. The mixture was diluted with water and extracted with ethyl acetate. Organic extracts were washed with 1% NaHCO₃ and brine, dried over anhydrous MgSO₄ and concentrated to provide 15.5 g of 113 as an oil. LCMS m/z 287 (M+H).

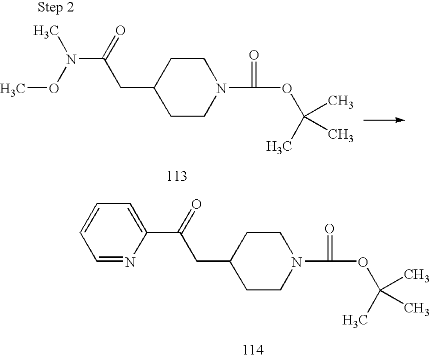

To a stirred solution of 2.9 g (18 mmol) of 2-bromopyridine in 30 mL of anhydrous. THF at −78° C. was added 7.5 mL of 2.5M solution of n-BuLi in hexane dropwise for 0.5 h. After being stirred at −78° C. for 1 h, the reaction mixture was added a solution of 5.1 g (17.8 mmol) of 113 in 15 mL of THF. The mixture was allowed to stir at ambient temperature for 48 h, mixed with saturated aquous NH₄Cl and extracted with ether. Organic extracts were washed with brine, dried over anhydrous MgSO₄ and concentrated to produce 5.7 g of 114 as an oil. LCMS m/z 305 (M+H).

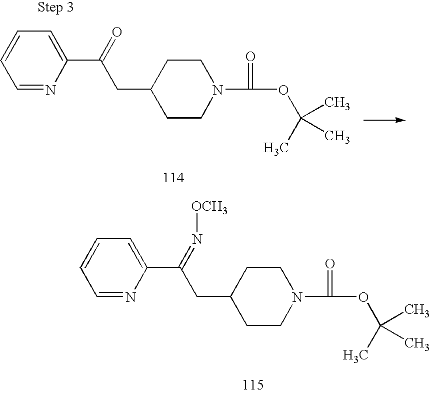

A solution of 3.15 g (10.4 mmol) of 114 and 3.47 g (41.6 mmol) of methoxylamine hydrochloride in 30 mL of ethanol and 30 mL of pyridine was heated under reflux for 18 h. The mixture was concentrated and the residue was taken up in ether and washed with water. The organic solution was dried over anhydrous MgSO₄ and concentrated to provide 2.5 g of 115 as an oil. LCMS m/z 334 (M+H).

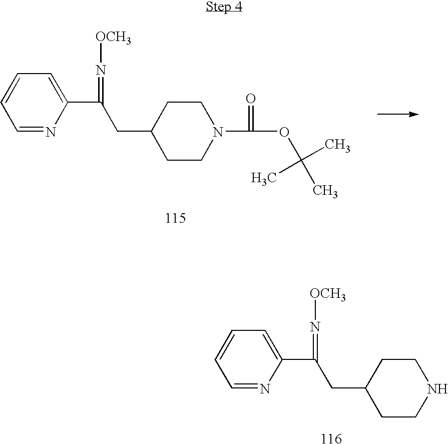

A solution of 2.4 g (7.2 mmol) of 22 in 20 mL of CH₂Cl₂ and 20 mL of trifluoroacetic acid was stirred at ambient temperature for 1 h. The solution was concentrated. The residue was basified with saturated aqueous NaHCO₃ and extracted with CH₂Cl₂. Organic extracts were washed with brine, dried over anhydrous MgSO₄ and concentrated to provide 1.41 g of 23 as a glass. LCMS m/z 234 (M+H).

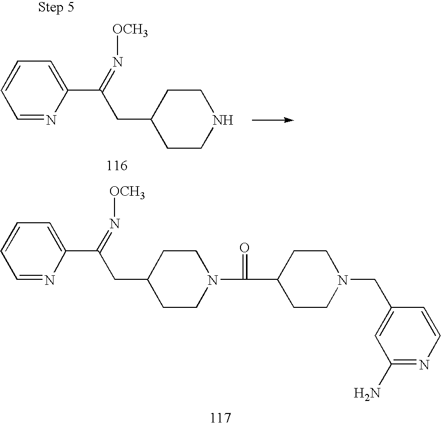

A mixture of 0.466 g (2 mmol) of 116, 0.517 g (2.2 mmol) of 5A, 0.276 g (2 mmol) of HOBt and 0.46 g (2.4 mmol) of DEC in 20 mL of anhydrous DMF was stirred at ambient temperature for 18 h. The mixture was concentrated by evaporation under reduced pressure at bath temperature of 25-45° C. and the residue was chromatographed with 4% (7N NH₃/CH₃OH) in CH₂Cl₂ to produce 0.48 g of syrup which was dissolved in 15 mL of EtAc-EtOH (3:1 v) and mixed with a solution of 0.26 g of maleic acid in 10 mL of EtAc-EtOH (1:1). The resulting precipitate was filtered to produce 0.35 g of the maleate salt of 117 (mp 160-163° C.). LCMS m/z 451 (M+H).

### Example 25

### Synthesis of Compound 121

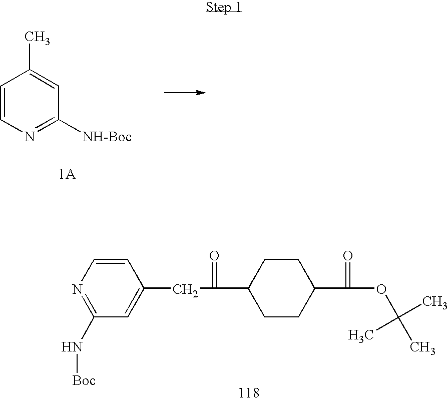

To a stirred solution of 4.16 g (20 mmol) of 1A in 80 mL of anhydrous THF at −78° C. was added dropwise 17 mL of 2.5M solution of n-BuLi in hexane for 25 minutes. After being stirred from −78° C. to room temperature for 1 h, the reaction mixture was added a solution of 6 g (22 mmol) of 26 in 100 mL of anhydrous THF and kept at room temperature for 18 h. The mixture was mixed with saturated aqueous NH₄Cl and extracted with EtAc. Organic extracts were washed with brine, dried over anhydrous MgSO₄ and concentrated to produce 6.1 g of 118 (mp 146-149° C.). LCMS m/z 420 (M+H).

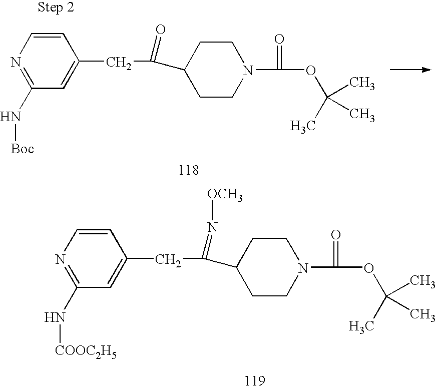

A solution of 3.71 g (8.8 mmol) of 118 and 3.7 g (44 mmol) of methoxylamine hydrochloride in 40 mL of pyridine and 40 mL of ethanol was heated under reflux for 2 days. The mixture was concentrated and the residue was taken up in CH₂Cl₂ and washed with saturated aqueous NaCl. Organic solution was dried over anhydrous MgSO₄ and concentrated to provide 2.6 g of 119 as a glass. LCMS m/z 421 (M+H).

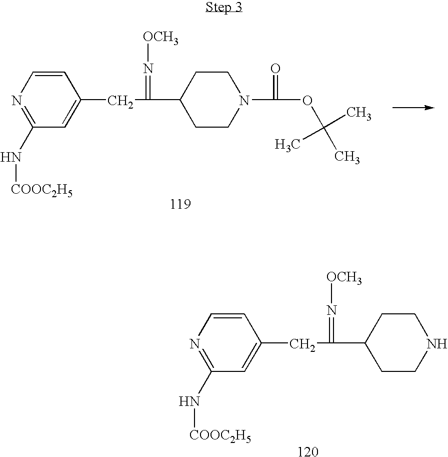

A solution of 0.9 g (2.14 mmol) of 119 in 10 mL of CH₂Cl₂ and 10 mL of trifluoroacetic acid was stirred at ambient temperature for 2 h. The solution was concentrated. The residue was taken up in CH₂Cl₂, washed with saturated NaHCO₃ and brine, dried over anhydrous MgSO₄ and concentrated to a solid residue which was triturated with CH₃CN and filtered to produce 0.29 g of 120 (mp 200-205° C.). LCMS m/z 321 (M+H).

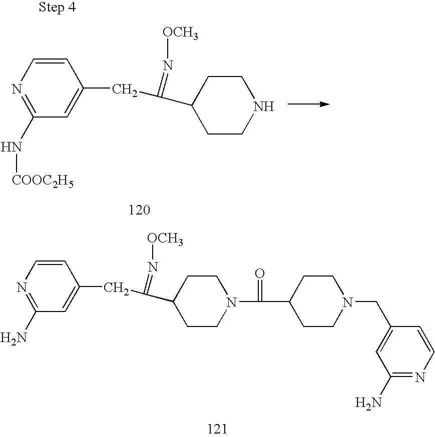

0.1 g (0.31 mmol) of 120 and 0.83 g (0.35) of 5A were coupled in the same manner as that described in Example 24, step 5 to produce 0.12 g of the maleate salt of 121 (mp 170-173° C.). LCMS m/z 538 (M+H).

### Example 26

### Synthesis of Compound 123

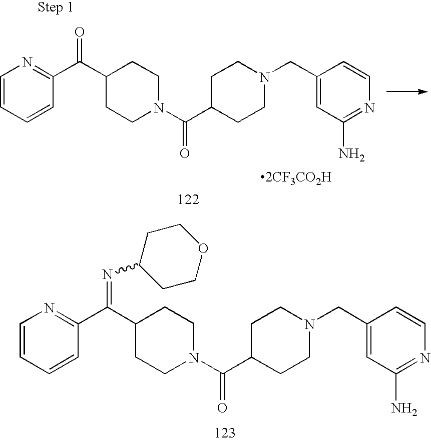

Using the method described in Example 6, step 7, compound 122 (0.26 g, 0.41 mmol) was converted to compound 123 (0.08 g, 40%).

### Example 27

### Synthesis of Compound 128

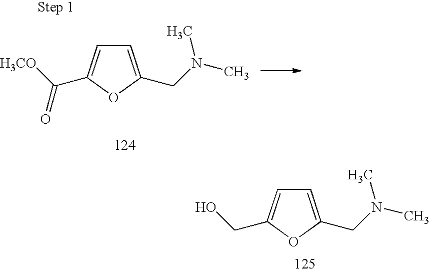

To a suspension of LAH (0.83 g, 22 mmol) in ether (20 mL) at 0° C. was added 124 (3.2 g, 17.5 mmol) in THF (15 mL) dropwise. The reaction was stirred at 0° C. for 1.5 h, and quenched by the addition of water (0.8 mL), 20% aqueous NaOH (0.8 mL), and water (2.4 mL). The mixture was stirred for 15 min and filtered and the filter cake washed with CH₂CL₂. The filtrate was concentrated to provide an oil which was dissolved in ether (30 mL) and washed with brine and dried (MgSO₄). Filtration and concentration in vacuo gave 125 (2.5 g) which was used without further purification.

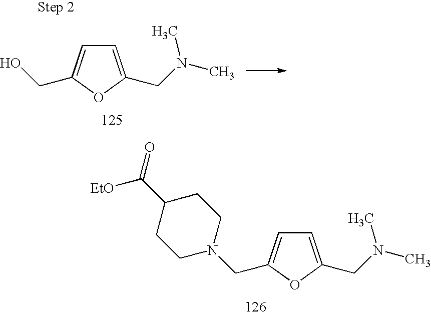

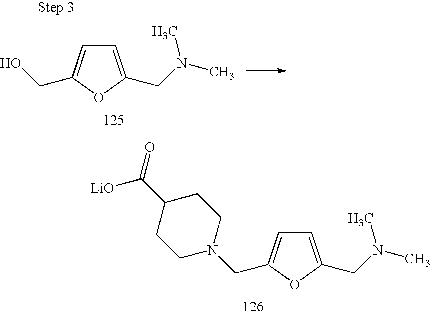

Using the method described in Example 22, step 3 and Example 1, steps 4, 5, and 6, compound 125 was converted to compound 126.

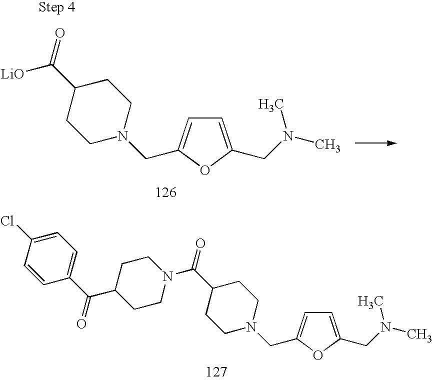

Using the method described in Example 6, step 5, compound 126 was converted to compound 127.

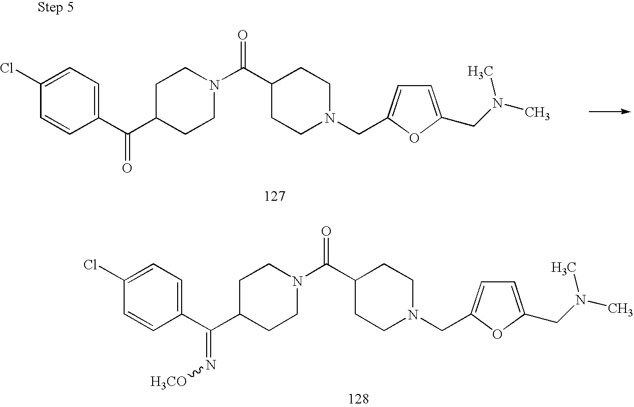

Using the method described in Example 6, step 7, compound 127 was converted to compound 128.

The compounds in Table 1 (first column) are prepared from the compounds in the last column of Table 1 by following essentially the same procedures as in the examples described above. In Table 1 “Cmpd. No.” stands for “Compound Number.”

TABLE 1

| Cmpd. | | Mass Spec. | |
|---|---|---|---|
| No. | STRUCTURE | [M + H]⁺ | Starting Material |
| 200 | 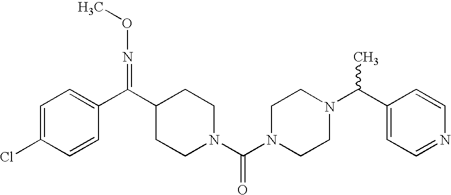 | 470.1 | 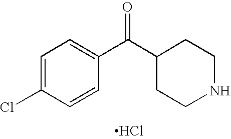 |
| 201 | 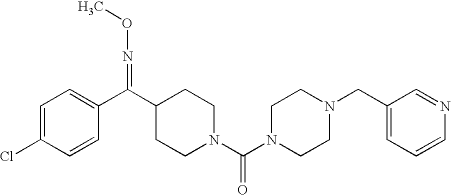 | 456.1 | 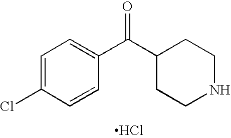 |
| 202 | 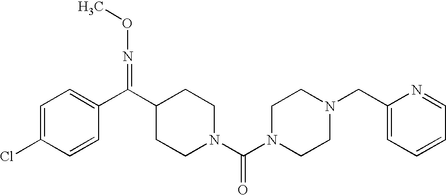 | 456.1 | 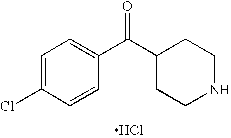 |
| 203 | 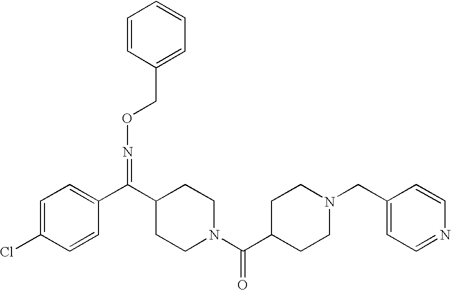 | 531.1 | 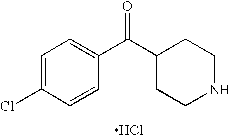 |
| 204 | 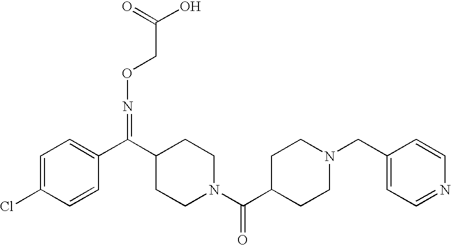 | 499.1 | 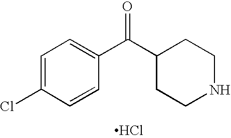 |
| 205 | 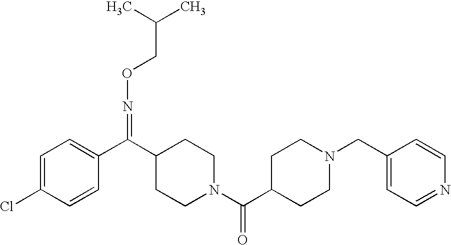 | 497.1 | 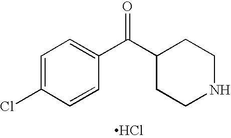 |
| 206 | 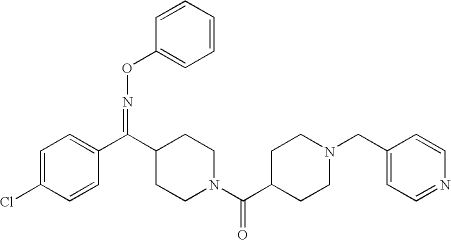 | 517.1 | 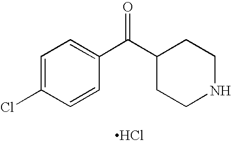 |
| 207 | 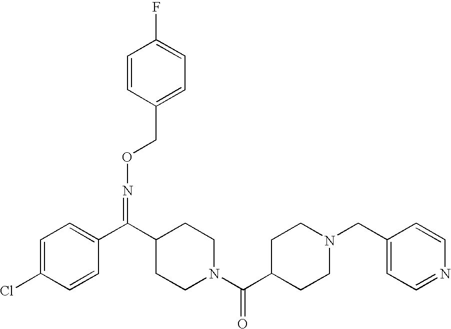 | 549.1 | 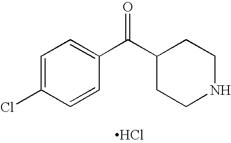 |
| 208 | 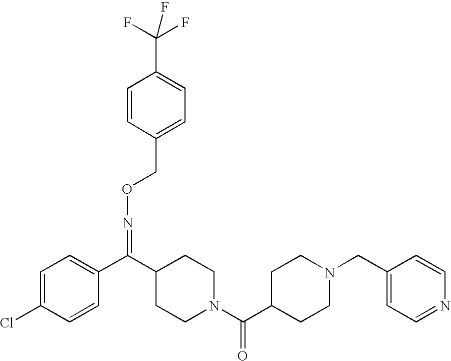 | 599.1 | 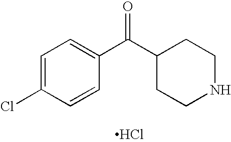 |
| 209 | 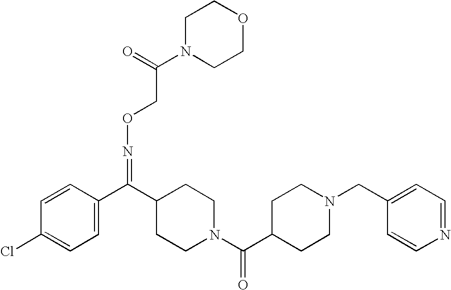 | 568.1 | 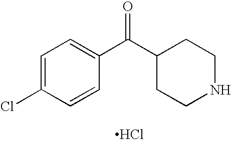 |
| 210 | 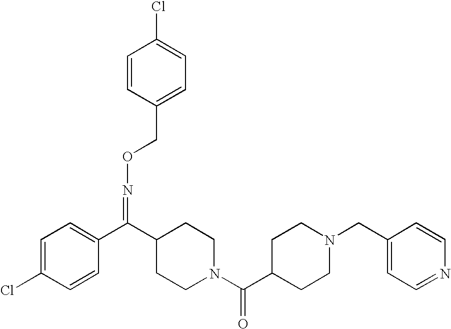 | 565.1 | 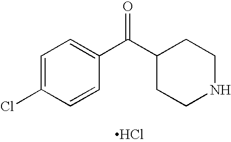 |
| 211 | 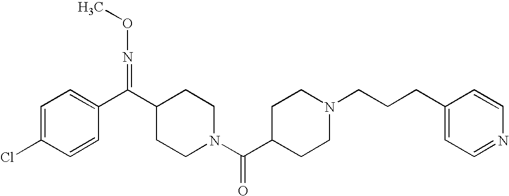 | 483 | 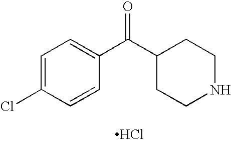 |
| 212 | 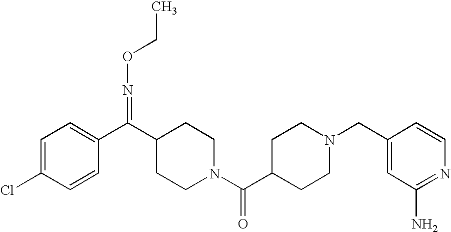 | 484.1 | 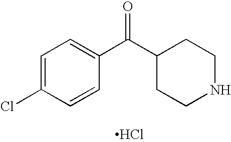 |
| 213 | 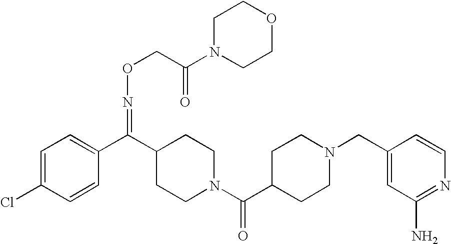 | 583.1 | 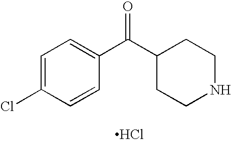 |
| 214 | 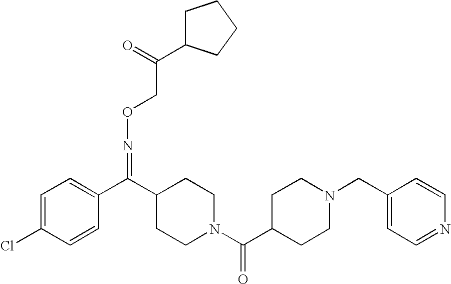 | 552.1 | 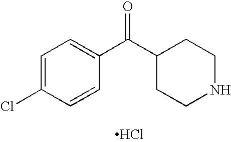 |
| 215 | 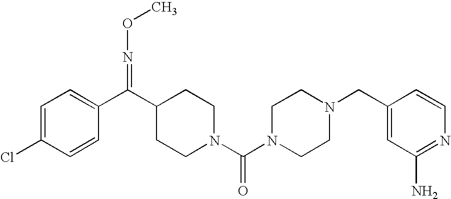 | 471 | 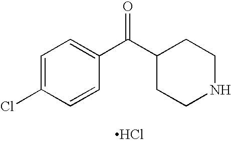 |
| 216 | 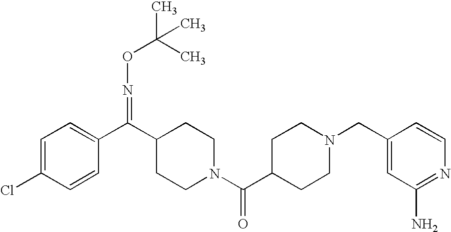 | 512 | 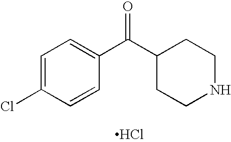 |
| 217 | 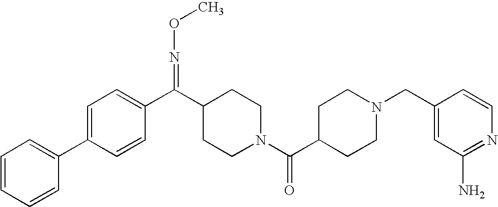 | 512 | 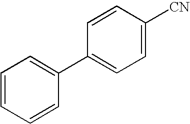 |
| 218 | 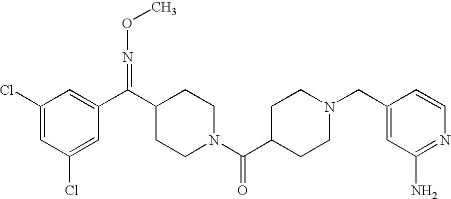 | 504 | 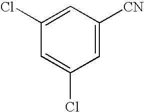 |
| 219 | 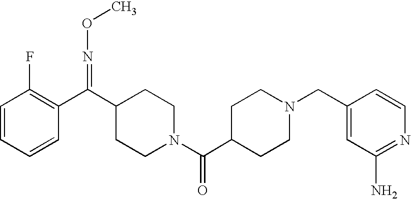 | 454 | 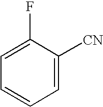 |
| 220 | 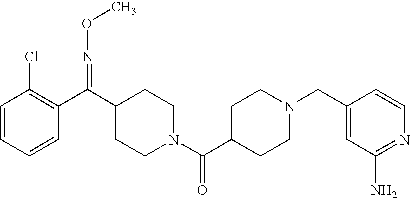 | 470 | 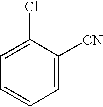 |
| 221 | 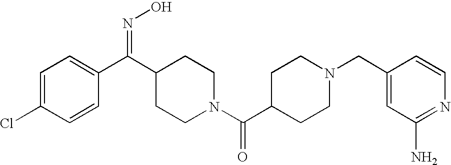 | 456 | 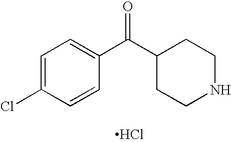 |
| 222 | 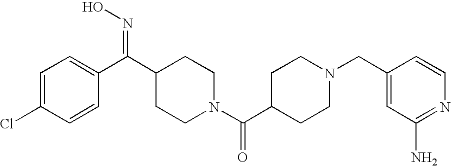 | 456 | 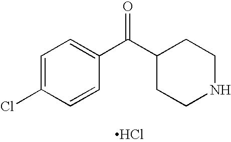 |
| 223 | 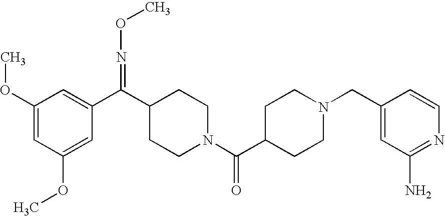 | 495 | 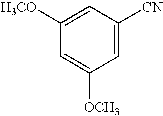 |
| 224 | 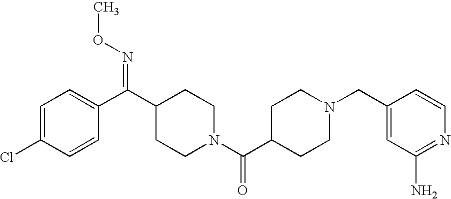 | 470 | 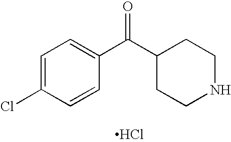 |
| 225 | 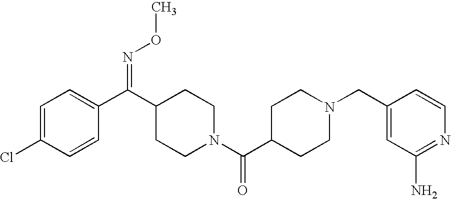 | 470 | 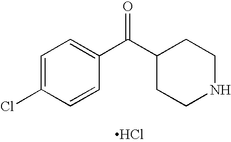 |
| 226 | 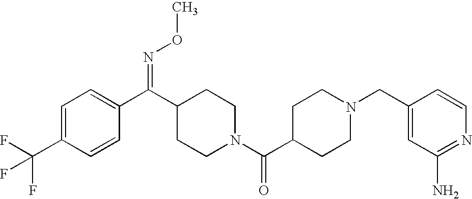 | 504 | 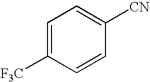 |
| 227 | 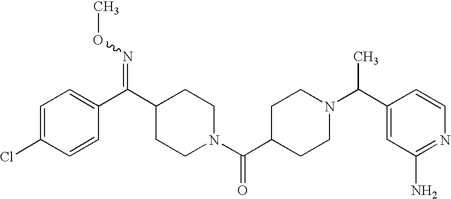 | 484 | 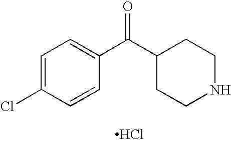 |
| 228 | 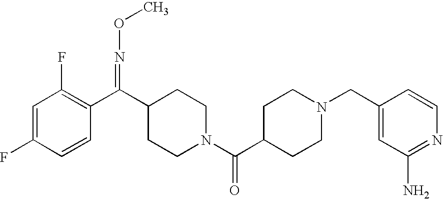 | 472 | 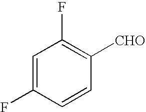 |
| 229 | 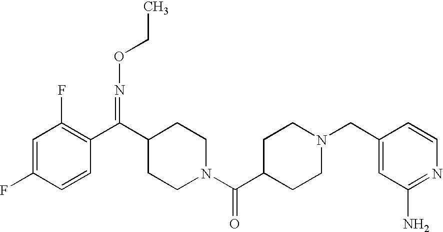 | 486 | 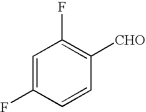 |
| 230 | 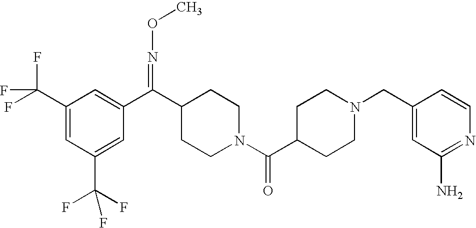 | 572 | 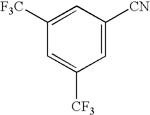 |
| 231 | 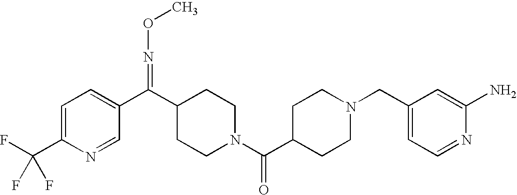 | 505 | 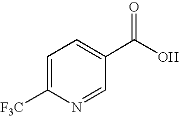 |
| 232 | 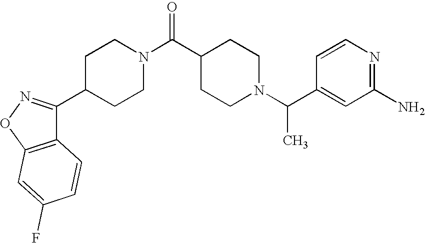 | 452 | 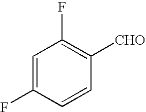 |
| 233 | 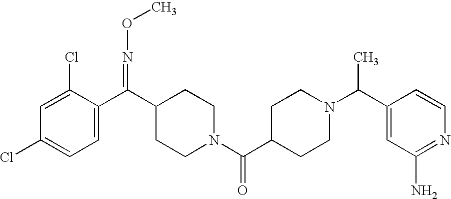 | 518 | 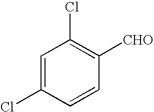 |
| 234 | 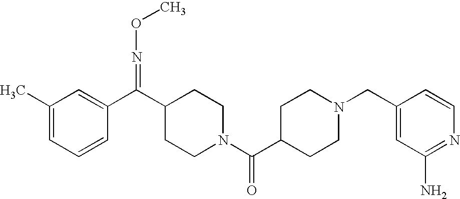 | 450 | 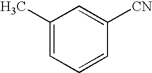 |
| 235 | 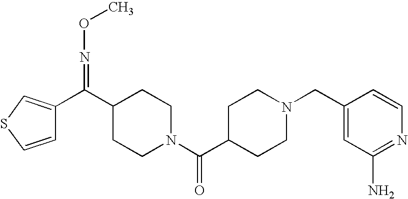 | 442 | 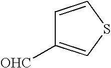 |
| 236 | 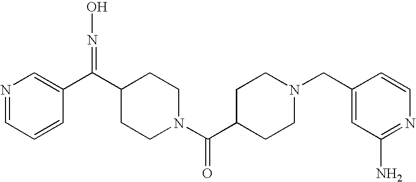 | 423 | 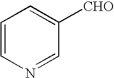 |
| 237 | 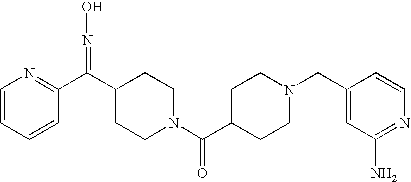 | 423 | 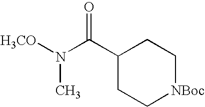 |
| 238 | 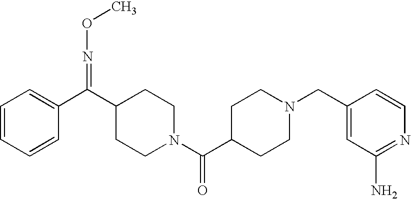 | 436 | 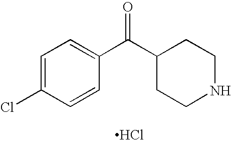 |
| 239 | 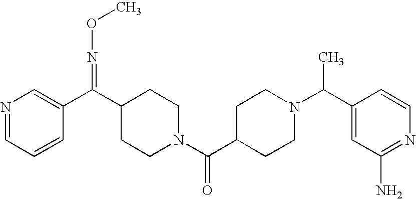 | 451 | 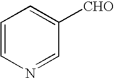 |
| 240 | 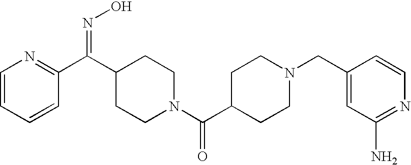 | 423 | 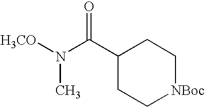 |
| 241 | 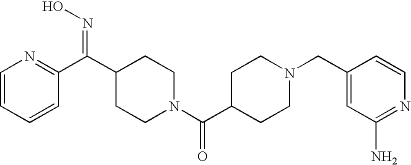 | 423 | 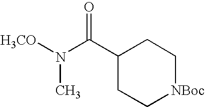 |
| 244 | 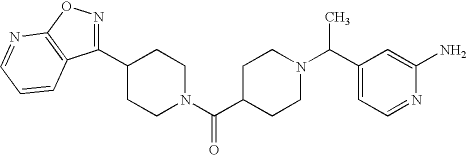 | 435 | 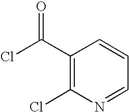 |
| 245 | 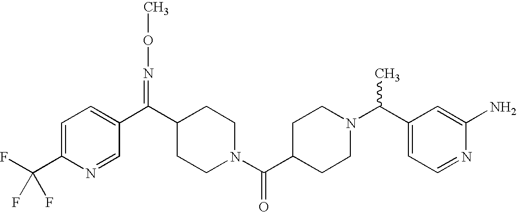 | 519 | 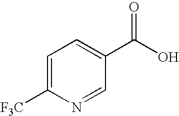 |
| 246 | 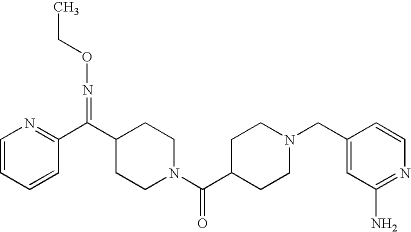 | 451 | 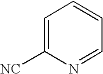 |
| 247 | 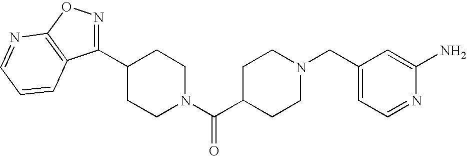 | 421 | 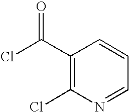 |
| 248 | 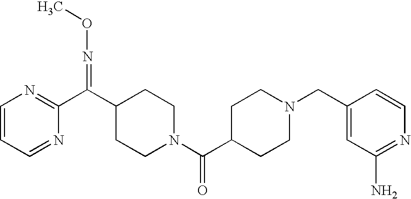 | 438 | 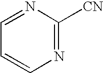 |
| 249 | 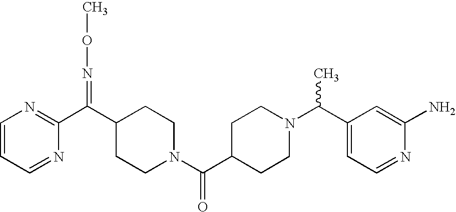 | 452 | 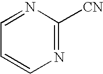 |
| 250 | 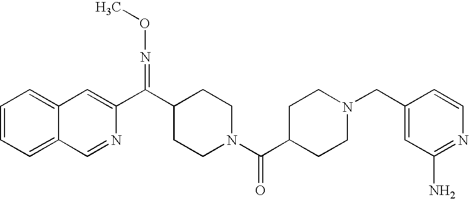 | 487 | 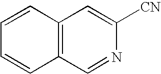 |
| 251 | 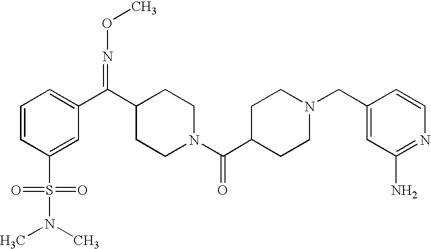 | 543 | 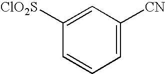 |
| 252 | 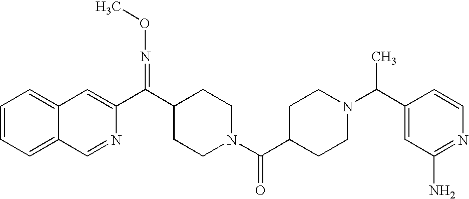 | 501 | 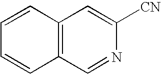 |
| 253 | 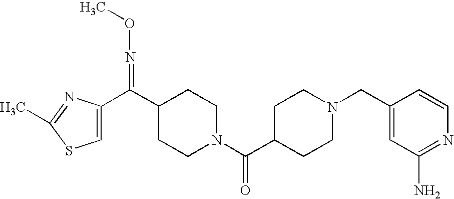 | 457 | 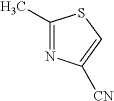 |
| 254 | 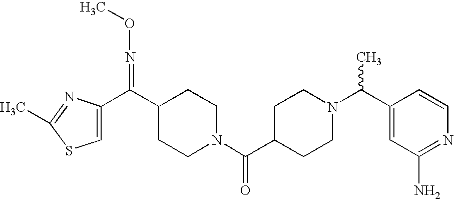 | 471 | 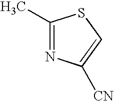 |
| 255 | 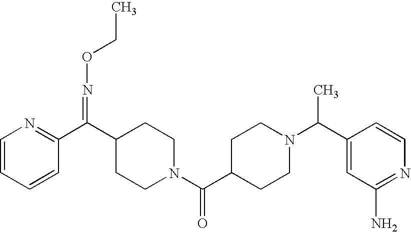 | 465 | 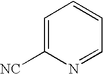 |
| 256 | 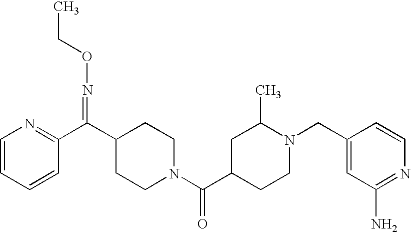 | 465 | 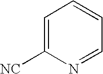 |
| 257 | 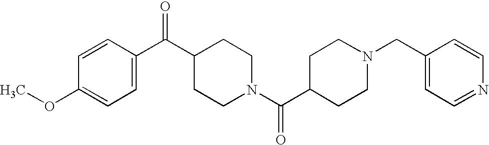 | 422 | 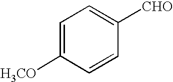 |
| 258 | 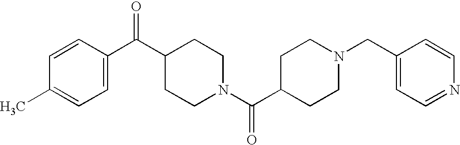 | 406 | 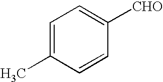 |
| 259 | 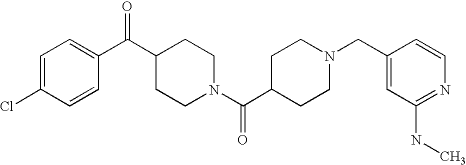 | 455 | 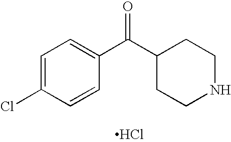 |
| 260 | 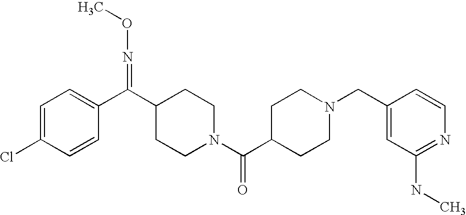 | 484 | 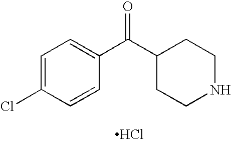 |
| 261 | 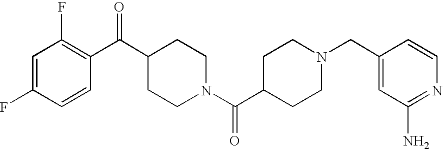 | 443 | 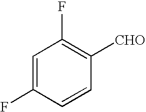 |
| 262 | 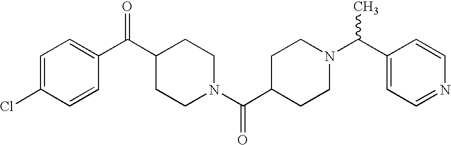 | 440 | 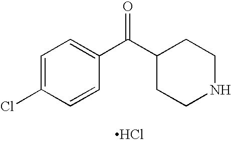 |
| 263 | 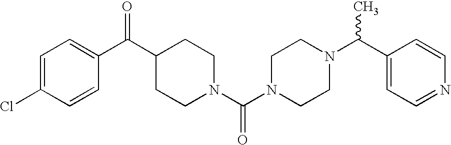 | 441 | 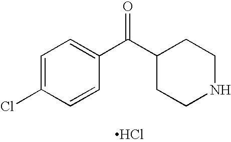 |
| 264 | 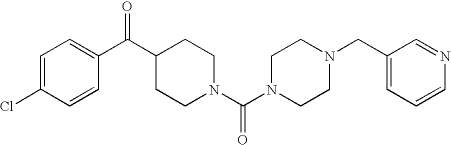 | 427 | 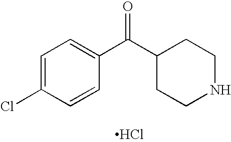 |
| 265 | 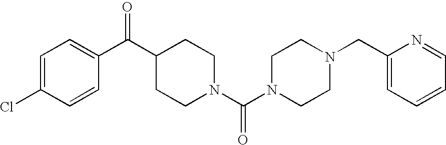 | 427 | 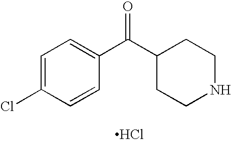 |
| 266 | 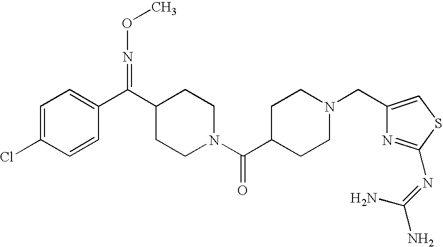 | 518 | 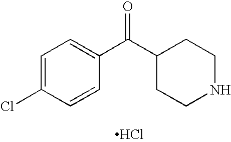 |
| 267 | 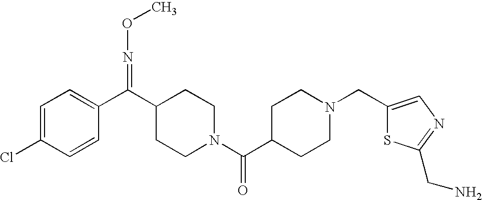 | 490 | 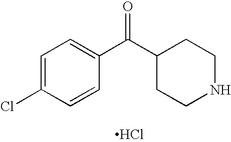 |
| 268 | 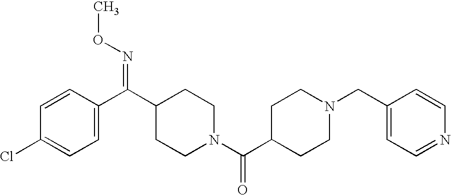 | 455 | 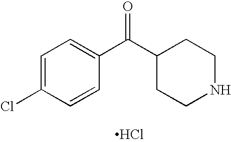 |
| 269 | 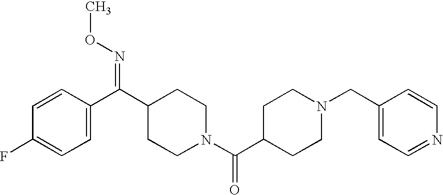 | 439 | 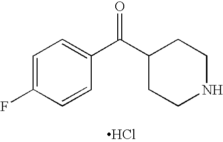 |
| 270 | 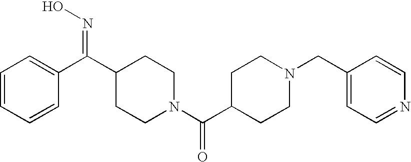 | 407 | 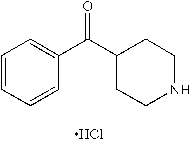 |
| 271 | 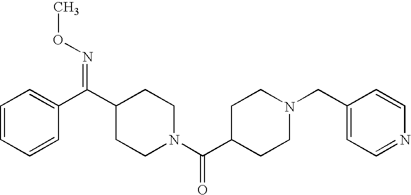 | 421 | 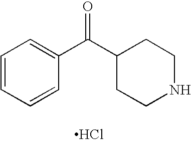 |
| 272 | 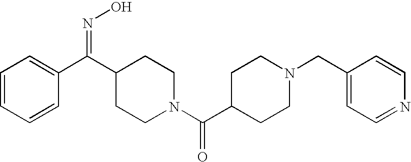 | 407 | 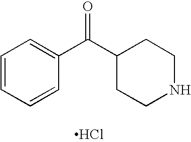 |
| 273 | 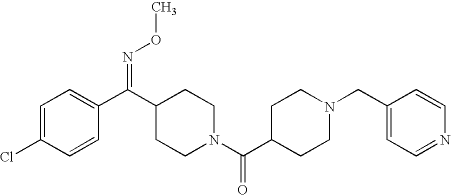 | 455 | 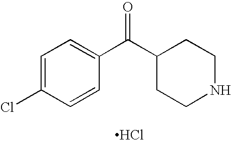 |
| 275 | 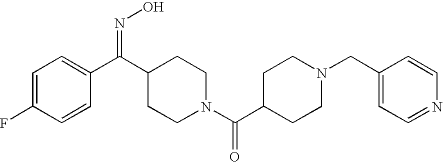 | 425 | 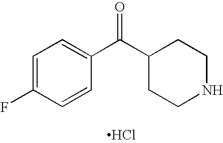 |
| 278 | 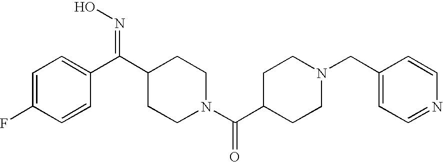 | 425 | 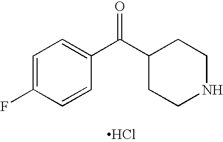 |
| 279 | 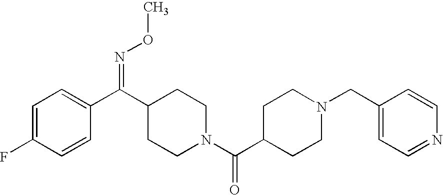 | 439 | 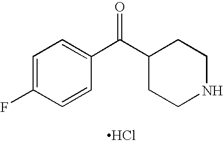 |
| 280 | 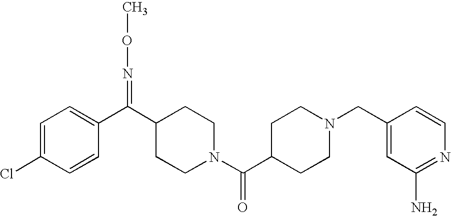 | 470 | 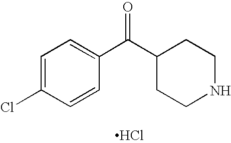 |
| 281 | 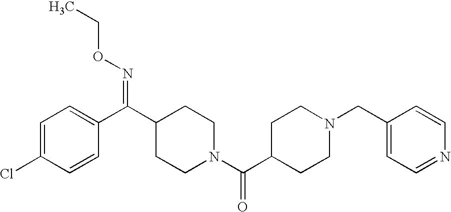 | 469 | 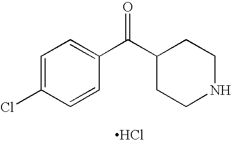 |
| 282 | 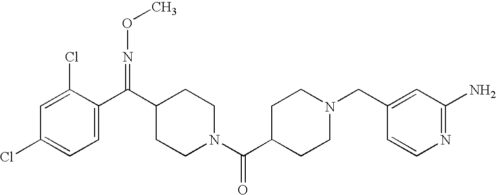 | 504 | 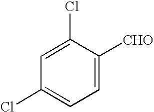 |

### Example 28

### Preparation of Compound 287

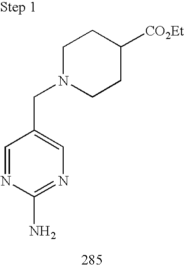

To a solution of 1.00 g (8.13 mmol) of pyrimidine aldehyde 67 (Step 2 of Example 13) in 40 ml of CH₂Cl₂ was added 1.36 mL (10.58 mmol) of ethyl isonipecotate and 2 drops of acetic acid. The mixture was stirred for 40 min. at room temperature, after which 2.58 g (12.17 mmol) of NaBH(OAc)₃ was added. The reaction mixture was then stirred for 20 h at room temperature, diluted with aqueous NaOH (pH adjusted to 11) and extracted with CH₂Cl₂. Organic phase was dried and concentrated, and the residue was flash chromatographed (4-8% ca. 3 N NH₃ in MeOH/CH₂Cl₂) to produce 1.55 g (5.87 mmol; 72%) of amine 285 as a yellowish solid.

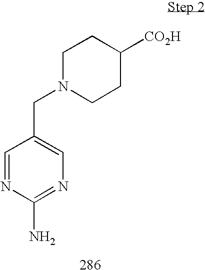

To a solution of 3.83 g (14.51 mmol) of ester 285 in 60 ml of 3:1:1 mixture of THF-MeOH—H₂O was added 1.22 g (29.02 mmol) of LiOH monohydrate. The reaction mixture was stirred at room temperature overnight, concentrated, and the residue was dried under high vacuum to produce 3.84 g of crude acid 286 lithium salt as a yellow solid. Material could be used directly or could be purified by passing through a silica gel plug eluting with ca. 3 N NH₃ in MeOH.

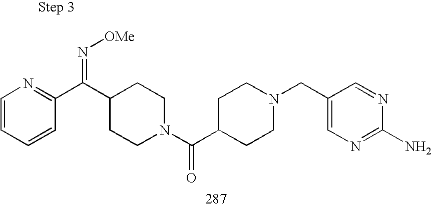

To a mixture of 3.32 g (14.05 mmol) of acid 286 and 4.07 g (14.05 mmol) of 4-[(E)-(methoxyimino)-2-pyridinylmethyl]piperidine dihydrochloride (see Compound 447 below) in 40 mL of DMF was added 8.94 mL (70.25 mmol) of 4-ethylmorpholine and 14.0 mL (23.52 mmol) of 50 wt. % solution of 1-propanephosphonic acid cyclic anhydride in ethyl acetate. The reaction mixture was stirred for 4.5 h at 50° C. followed by 14 h at room temperature. Concentration of the mixture was followed by exposure to high vacuum for 24 h to remove remaining DMF. The residue was partitioned between aqueous NaOH and CH₂Cl₂, organic phase was separated, dried and concentrated, and the residue was flash chromatographed (5-15% ca. 3 N NH₃ in MeOH/CH₂Cl₂) to produce 4.60 g (10.51 mmol; 75%) of amide 287 as a light tan foam. MS 438 (M+1).

### Example 29

### Preparation of Compound 296

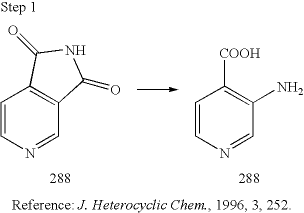

3,4 Pyridine-dicarboximide 288 (10.0 g; 67.5 mmoles) was dissolved in 162 g. of 10% aqueous NaOH and the solution was cooled to an internal temperature of 7° C. in an ice-salt bath. Bromine (3.6 ml; 70 mmoles) was added dropwise. After the addition, the solution was heated for 45 minutes at a bath temperature of 80-85° C. The yellow solution was then cooled. to an internal temperature of 37° C., then 17 ml of glacial acetic acid were added dropwise to a pH of 5.5. The resulting mixture was saved overnight in a refrigerator. The solid formed was filtered and washed with 5 ml of water and 5 ml of methanol. The reaction yielded 6.35 g. of product 289 melting at 280-285° C. (decomp.).

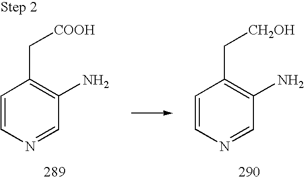

Solid Compound 289 (9.5 gr.; 69 mmoles) was carefully added in three aliquots to a slurry of lithium aluminum hydride (9.5 gr.; 250 mmoles) in 200 ml of dry tetrahydrofuran. The resulting hot mixture was stirred at room temperature for two days. After cooling in an ice bath, the reaction was quenched with very careful sequential dropwise addition of 10 ml of water, followed by 10 ml of 15% aqueous NaOH, then by 30 ml of water. The resulting solid was filtered through a pad of Celite and washed several times with THF. The oil obtained after evaporation of the solvent, solidified on standing. The reaction mixture was purified by flash chromatography on silica gel using 5% MeOH(NH₃)/EtOAc as eluent yielding 6.21 (72%) of Compound 290. LC-MS: m/z=125 (M+1).

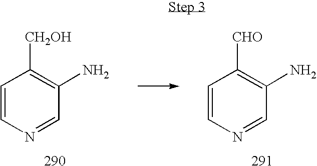

Manganese dioxide (29 gr.; 334 mmoles) was added, in one portion, at room temperature, to a suspension of 3-amino-4-hydroxymethyl pyridine 290 (5.0 gr.; 40.3 mmoles) in 500 ml of chloroform with good stirring. After two days, the solid is filtered through a pad of Celite and washed with chloroform. Removal of the solvent using reduced pressure yielded 4.2 grams (85%) of Compound 291 as a yellow solid.

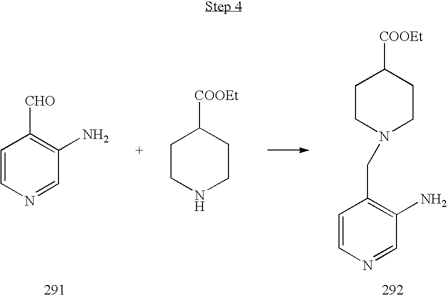

A dry dichloromethane (400 ml) solution of ethyl isonipecotate (12.5 gr.; 79.5 mmoles) and 3-amino pyridine 4-carboxyaldehyde 291 (3.33 gr.; 27.3 mmoles) was stirred at room temperature for one hour, then 60 grams of activated 3 Å molecular sieves were added. The mixture was stirred for an additional 90 minutes, then 20 grams (96.4 mmoles) of sodium triacetoxy borohydride was added at room temperature in one portion. After stirring for three days, the solid was filtered through a pad of Celite and washed with dichloromethane. The solution was stirred for 15 minutes with 100 ml of saturated aqueous sodium bicarbonate then separated from the aqueous layer. The organic layer was washed two more times with saturated aqueous sodium bicarbonate, then with brine and dried with anhydrous sodium sulfate. After evaporation of the solvent, the resulting oil was purified by flash chromatography on silica gel using EtOAc:Hexanes:MeOH(NH₃) as eluent. The procedure yielded 6.8 gr. (94%) of Compound 292. FAB-MS: m/z=264 (M+1).

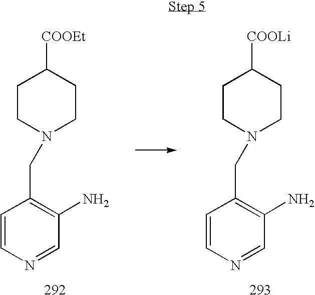

Ethyl 1-[(3-amino-4-pyridinyl)methyl]-4-piperidinecarboxylate 292 (4.75 gr.; 18.04 mmoles) was stirred for 24 hours at room temperature with 1.51 gr. (36 mmoles) of lithium hydroxide monohydrate in 75 ml of methanol. Removal of the solvent using reduced pressure yielded Compound 293 as a white solid.

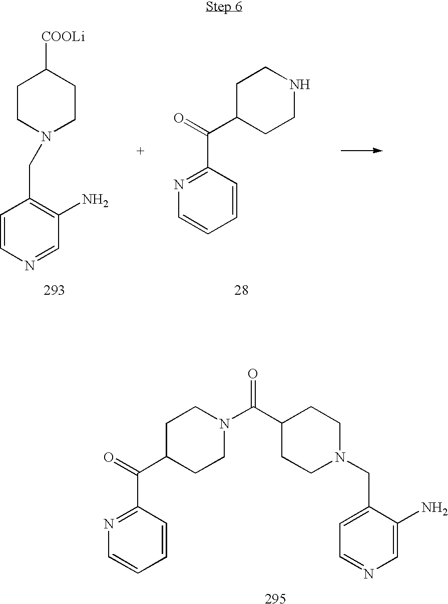

4-(2-pyridinylcarbonyl)piperidine 28 (Step 4 in Example 6) (0.3 gr.; 1.58 mmoles), lithium 1-[(3-amino-4-pyridinyl)methyl]-4-piperidinecarboxylate 293 (0.34 gr.; 1.4 mmoles), DEC (0.38 gr.; 2.0 mmoles), and HOBT (0.27 gr.; 2.0 mmoles) were stirred at room temperature in 10 ml of dry DMF for two days. The reaction was quenched with 50 ml. of 0.5 N aqueous NaOH, then the solution was extracted with dichloromethane. The combined extracts were washed with brine and dried over anhydrous sodium sulfate. The product 295 was isolated by flash chromatography on silica gel using EtOAc:Hexanes:MeOH(NH₃) (50:45:5) as eluent. Yields: 0.27 gr. (47%). FAB-MS: m/z=408 (M+1).

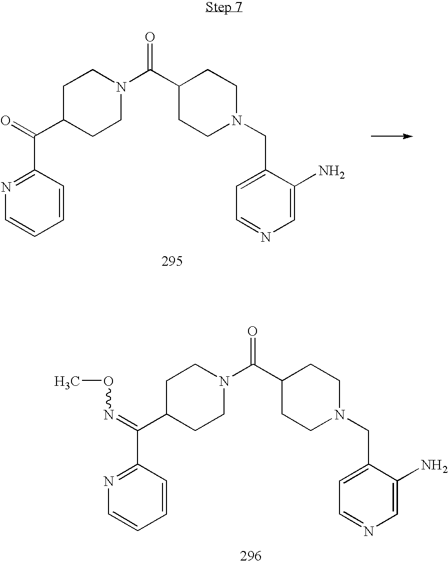

1-[[[1-[(3-amino-4-pyrimidinyl)methyl]-4-piperidinyl]carbonyl]-4-(2-pyridinylcarbonyl)piperidine 295 (0.196 gr.; 0.48) and methoxyamine hydrochloride (0.401 gr. 4.8; mmoles) were heated, under N₂, at a bath temperature of 70° C. for 24 hours in 6.0 ml of dry pyridine. After removing the pyridine using reduced pressure, the residue was treated with saturated aqueous sodium bicarbonate. The resulting mixture was extracted several times with dichloromethane. The combined extracts were washed with brine and dried over anhydrous sodium sulfate. The reaction mixture was purified by silica gel preparative thin layer chromatography. The plates were eluted with EtOAc:Hexanes:MeOH(NH₃) (60:35:5) and the product 296 was extracted with 10% MeOH(NH3)/EtOAc. Yields: 0.15 gr. (71%). FAB-MS: m/z=437 (M+1).

### Example 30

### Preparation of Compound 301

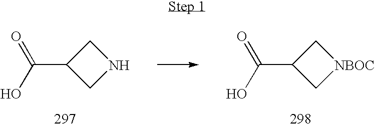

A mixture of 297 (1 g, 10 mmol) in 1:1 water-dioxane (50 mL) was treated with Et₃N (4 mL, 13 mmol) and BOC₂O (2.8 g, 13 mmol) at 4° C. and allowed to warm to 20° C. for one day. The solvent was then removed in vacuo. The residue was taken up in 1:1 water-ethyl acetate and the organic layer was discarded. The aqueous layer was acidified with 1 N aqueous HCl and extracted three times with ethyl acetate. The combined organic phases were washed with water and brine, dried (Na₂SO₄), and concentrated to give 298 as a white solid (1.8 g, 90%).

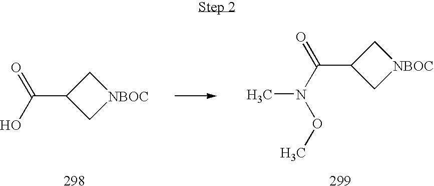

A mixture of 298 (1.8 g, 9 mmol), N,O-dimethylhydroxylamine hydrochloride (2.6 g, 27 mmol), EDCI (5 g, 27 mmol), HOBt (0.1 g, 1 mmol), and DIPEA (12.5 mL, 72 mmol) in DMF (30 mL) was stirred at 20° C. overnight. The reaction was then concentrated to half volume in vacuo, poured onto water, and extracted three times with ethyl acetate. The combined organic phases were washed with saturated aqueous NH₄Cl, saturated aqueous NaHCO₃, water and brine, dried (Na₂SO₄), and concentrated to give 299 as a clear oil (2.1 g, 98%).

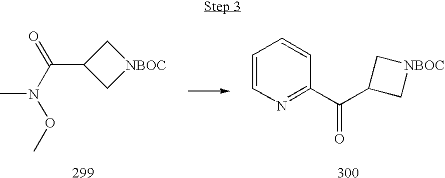

To a solution of 2-bromopyridine (1.2 mL, 12 mmol) in THF (60 mL) at −78° C. was added n-BuLi (8 mL of a 1.6 M solution in hexanes, 12 mmol) dropwise over 15 min. After stirring for an additional 30 min at −78° C., a solution of 299 (1 g, 4 mmol) in THF (20 mL) was slowly added. The reaction was then heated to 60° C. for 1 h. After cooling to 20° C., the reaction was diluted with ether, quenched with saturated aqueous Na₂SO₄, and dried with solid Na₂SO₄. The mixture was filtered through a plug of solid Na₂SO₄ and concentrated in vacuo. Flash column chromatography (0-20% ethyl acetate-hexanes) yielded 300 as a yellow oil (0.12 g, 11%).

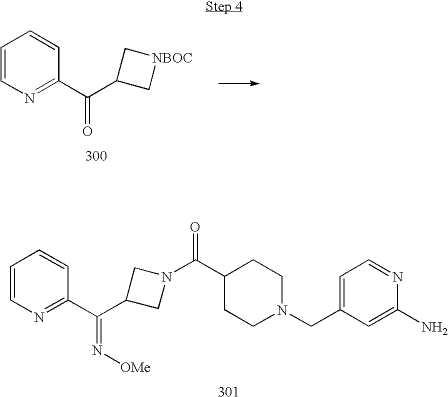

Following procedures similar to those of Steps 4 to 7 of Example 6, compound 301 was obtained. MS 409 (M+1).

Following procedures similar to those described in the examples above, the compounds in Table 2 were prepared.

TABLE 2

| | | MS |
|---|---|---|
| Compound | STRUCTURE | (M + 1) |
| 302 | 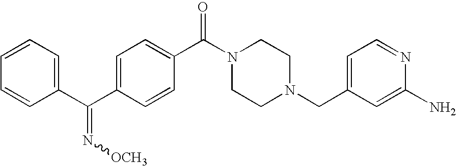 | 430 |
| 303 | 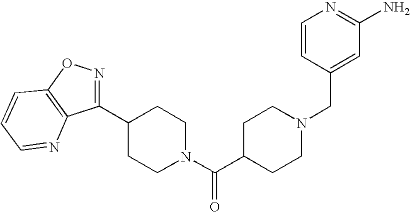 | 421 |
| 304 | 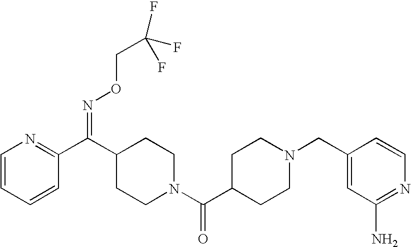 | 505 |
| 305 | 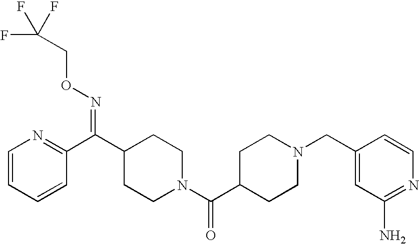 | 505 |
| 306 | 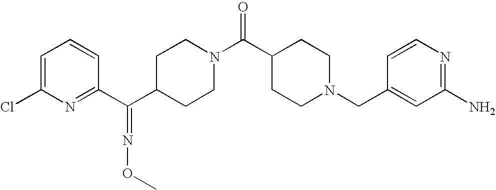 | 471 |
| 307 | 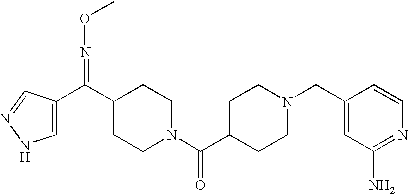 | 426 |
| 308 | 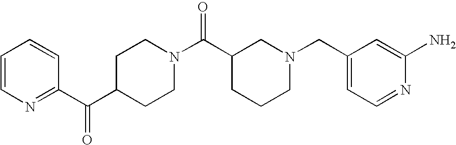 | 408 |
| 309 | 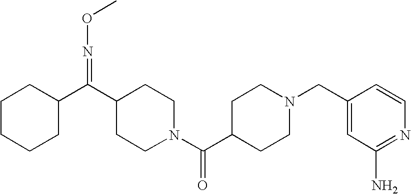 | 442 |
| 310 | 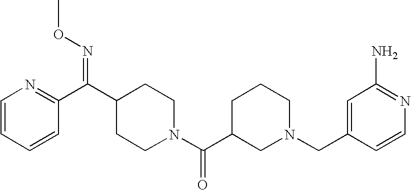 | 437 |
| 311 | 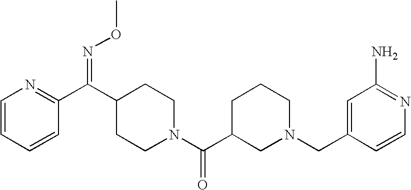 | 437 |
| 312 | 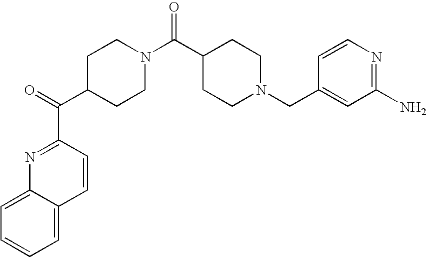 | 458 |
| 313 | 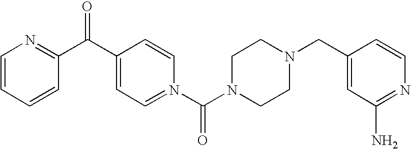 | 402 |
| 314 | 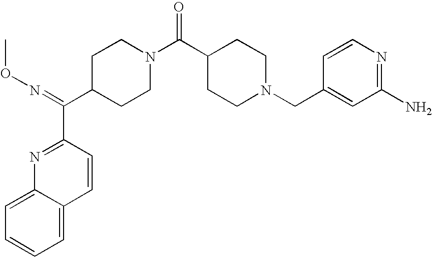 | 487 |
| 315 | 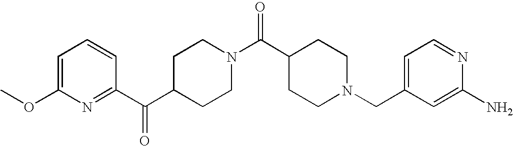 | 438 |
| 316 | 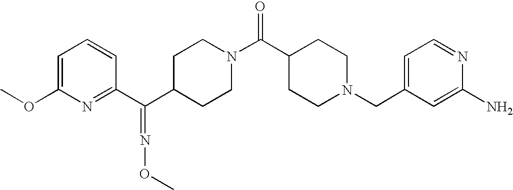 | 467 |
| 317 | 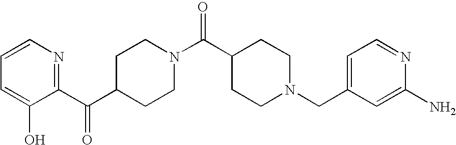 | 424 |
| 318 | 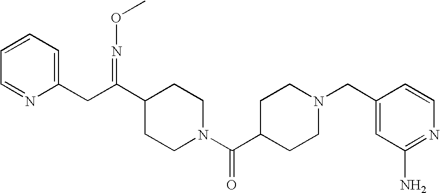 | 451 |
| 319 | 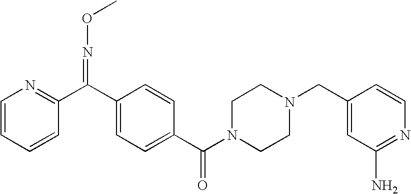 | 430 |
| 320 | 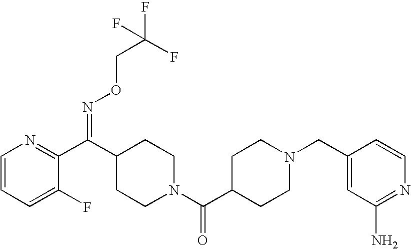 | 523 |
| 321 | 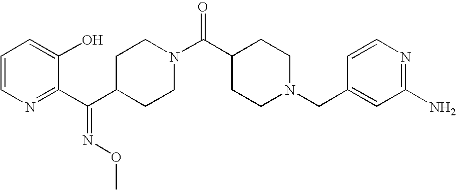 | 453 |
| 322 | 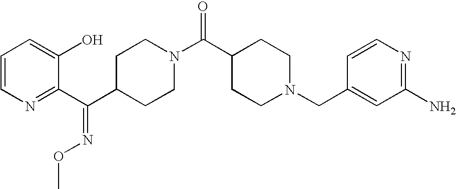 | 453 |
| 323 | 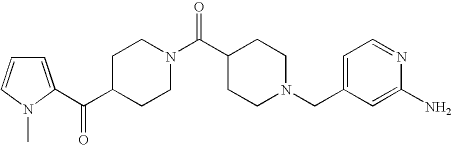 | 410 |
| 324 | 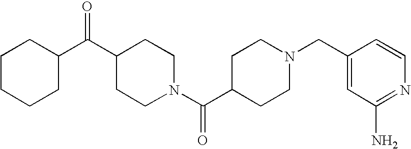 | 413 |
| 325 | 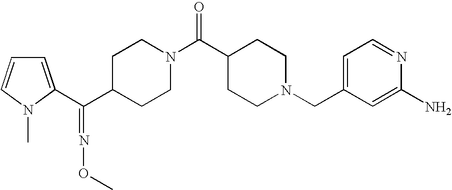 | 439 |
| 326 | 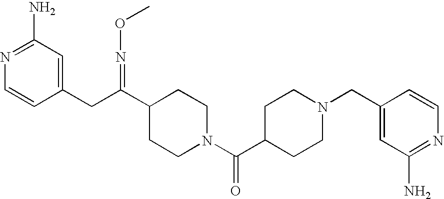 | 466 |
| 327 | 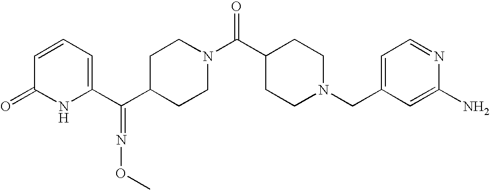 | 453 |
| 328 | 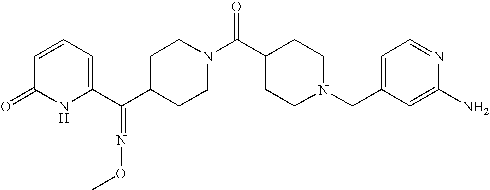 | 453 |
| 329 | 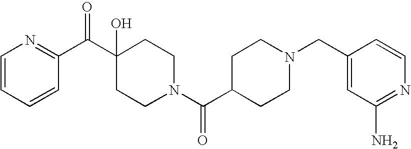 | 424 |
| 330 | 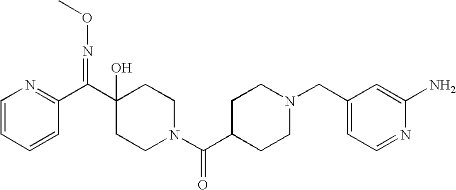 | 453 |
| 331 | 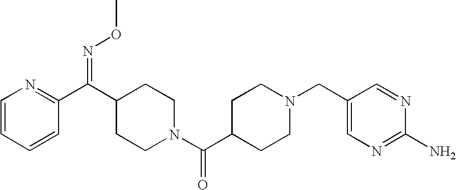 | 438 |
| 332 | 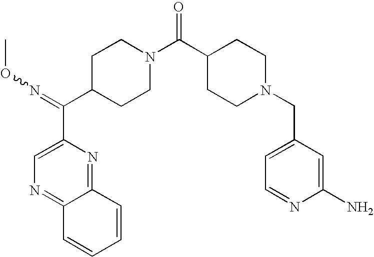 | 488 |
| 333 | 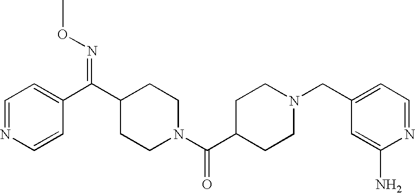 | 437 |
| 334 | 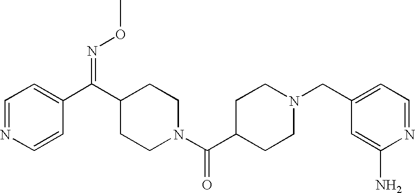 | 437 |
| 335 | 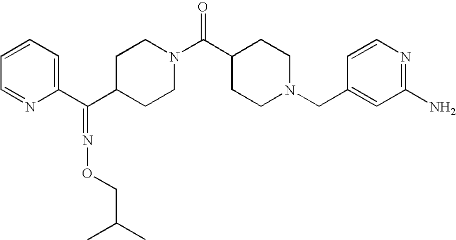 | 479 |
| 336 | 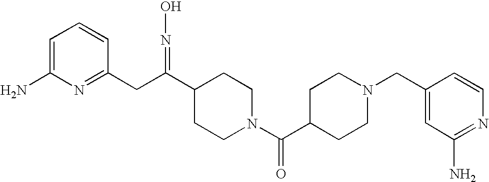 | 452 |
| 337 | 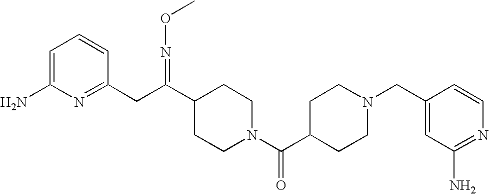 | 466 |
| 338 | 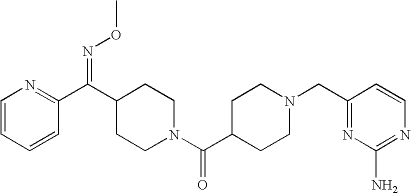 | 438 |
| 339 | 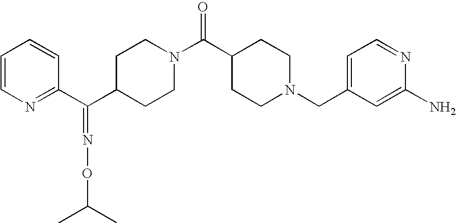 | 465 |
| 340 | 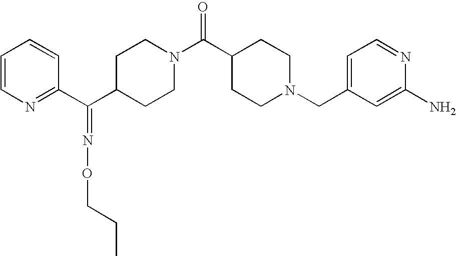 | 465 |
| 341 | 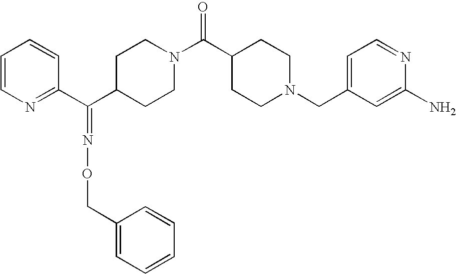 | 513 |
| 342 | 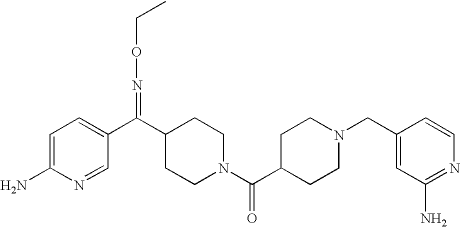 | 452 |
| 343 | 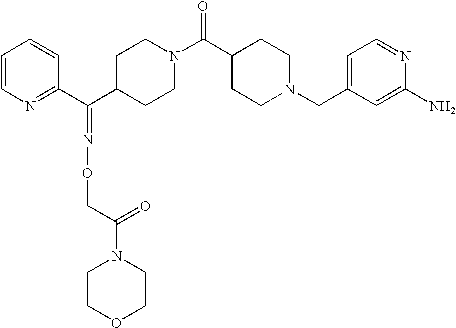 | 550 |
| 344 | 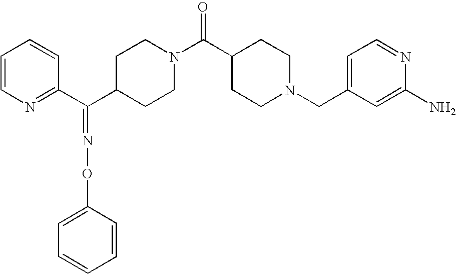 | 499 |
| 345 | 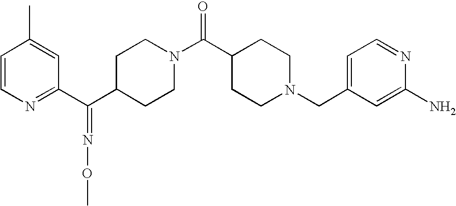 | 451 |
| 346 | 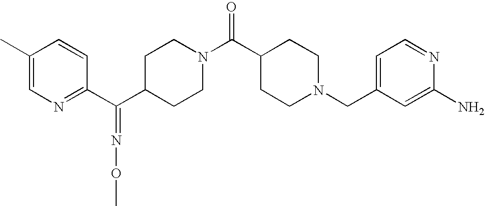 | 451 |
| 347 | 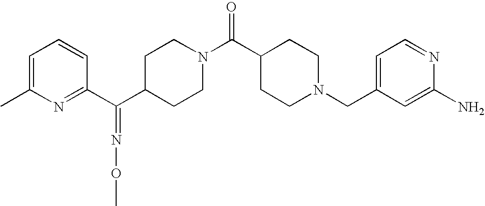 | 451 |
| 348 | 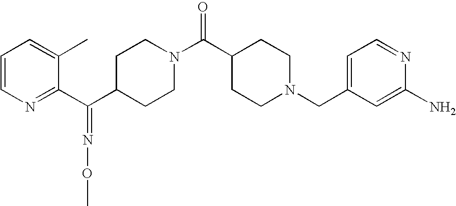 | 451 |
| 349 | 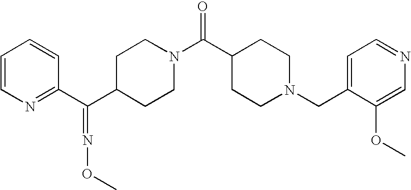 | 452 |
| 350 | 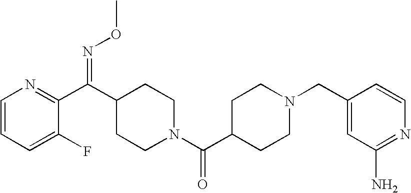 | 455 |
| 351 | 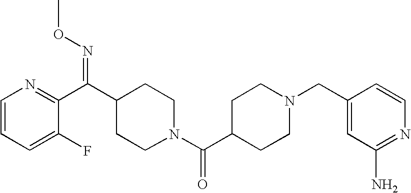 | 455 |
| 352 | 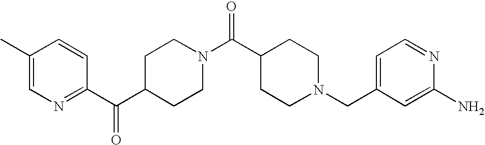 | 422 |
| 353 | 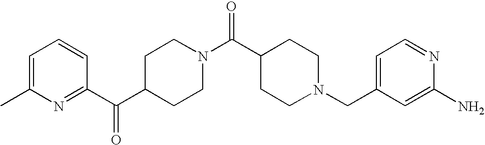 | 422 |
| 354 | 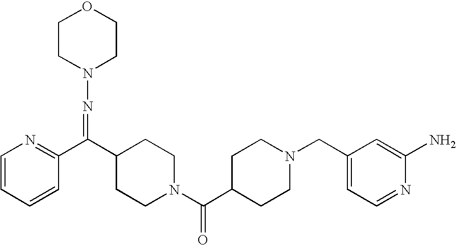 | 492 |
| 355 | 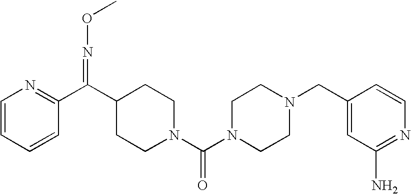 | 438 |
| 356 | 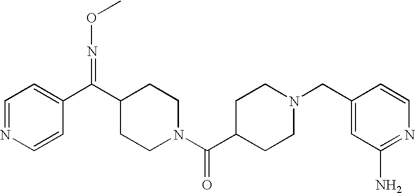 | 437 |
| 357 | 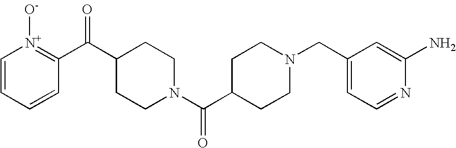 | 424 |
| 358 | 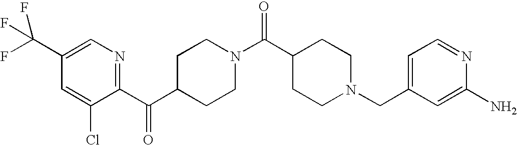 | 510 |
| 359 | 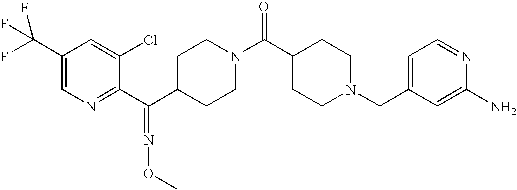 | 539 |
| 360 | 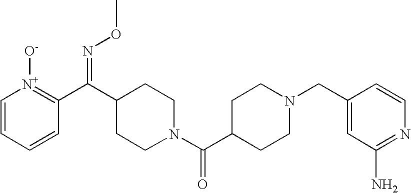 | 453 |
| 361 | 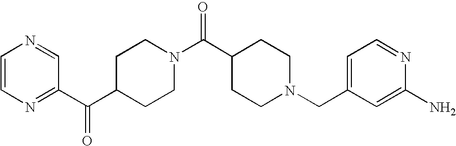 | 409 |
| 362 | 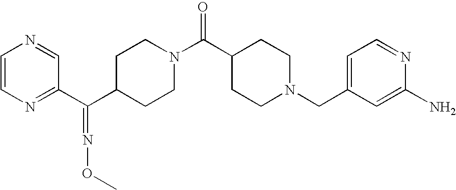 | 438 |
| 363 | 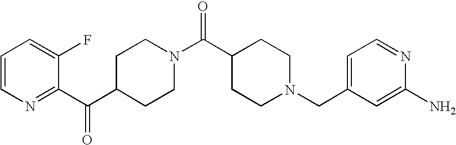 | 426 |
| 364 | 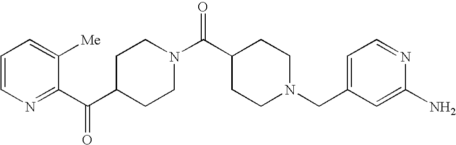 | 422 |
| 365 | 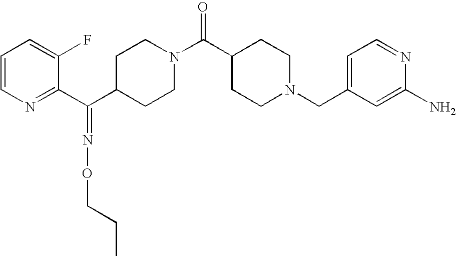 | 483 |
| 366 | 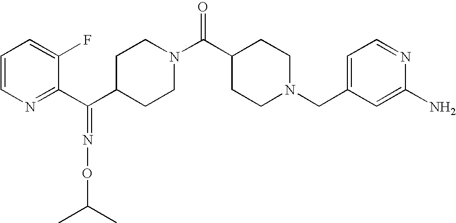 | 483 |
| 367 | 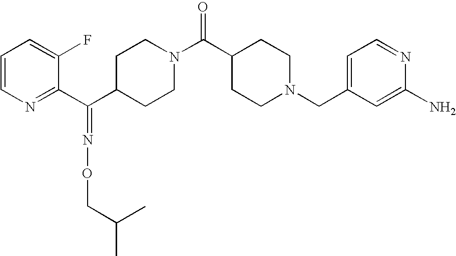 | 497 |
| 368 | 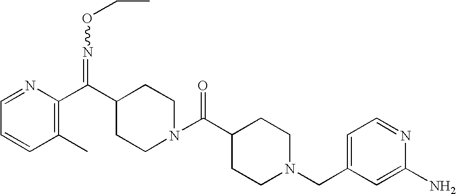 | 465 |
| 369 | 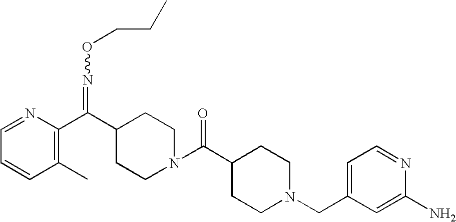 | 479 |
| 370 | 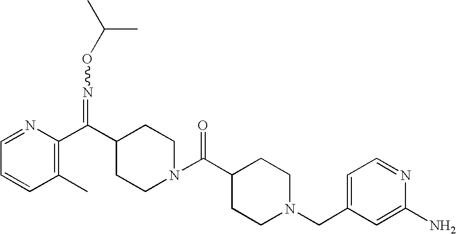 | 479 |
| 371 | 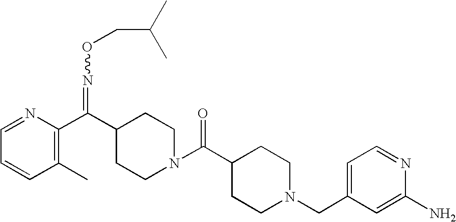 | 493 |
| 372 | 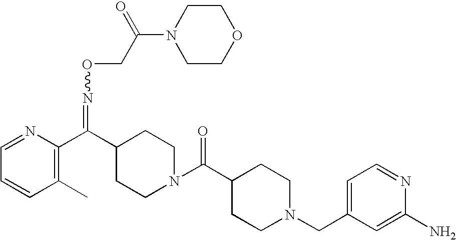 | 564 |
| 373 | 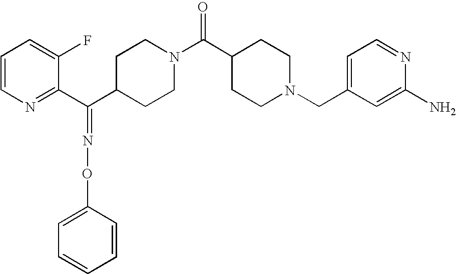 | 517 |
| 374 | 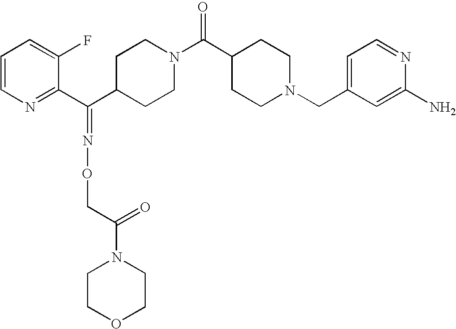 | 568 |
| 375 | 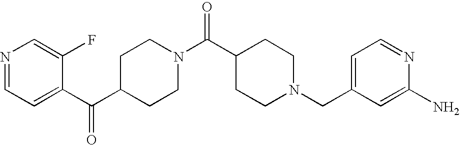 | 426 |
| 376 | 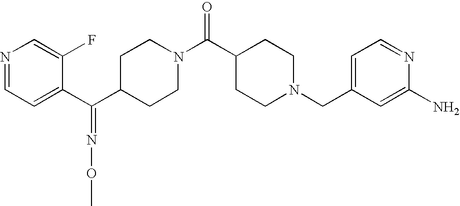 | 455 |
| 377 | 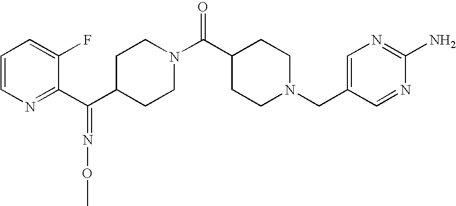 | 456 |
| 378 | 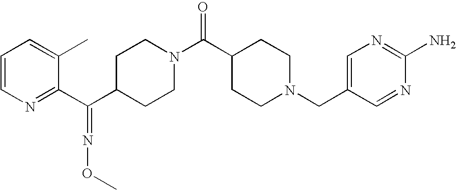 | 452 |
| 379 | 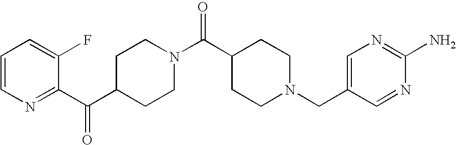 | 427 |

If one were to follow procedures similar to those described in the examples above, the compounds in the “Structure” column of Table 3 would be obtained using the starting material listed in Table 3. Each compound in Table 3 is a mixture of oxime isomers, as represented by the [symbol] bond between the oxime nitrogen and the OH or OCH₃ moiety. In Table 3 “CMPD” stands for “Compound”.

TABLE 3

| CMPD | Structure | Starting Material |
|---|---|---|
| 380 | 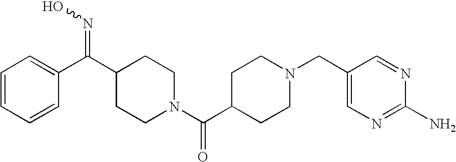 | 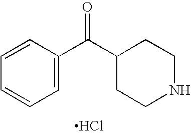 |
| 381 | 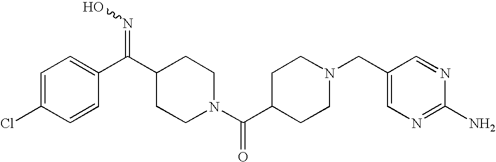 | 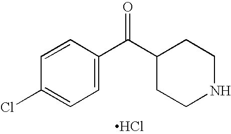 |
| 382 | 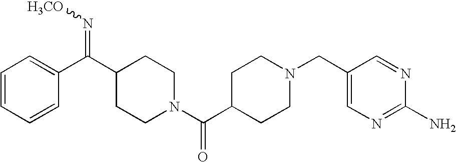 | 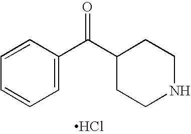 |
| 383 | 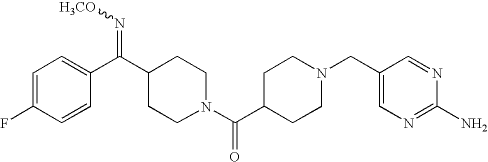 | 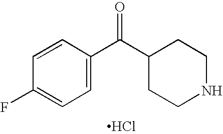 |
| 384 | 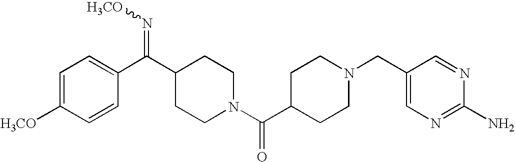 | 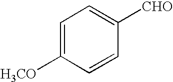 |
| 385 | 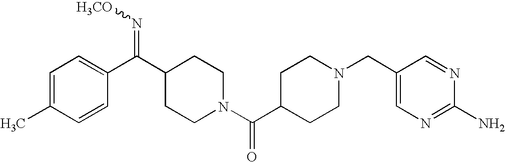 | 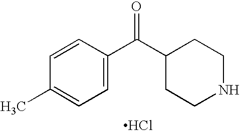 |
| 386 | 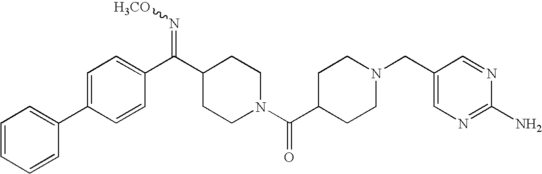 | 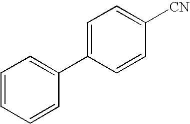 |
| 387 | 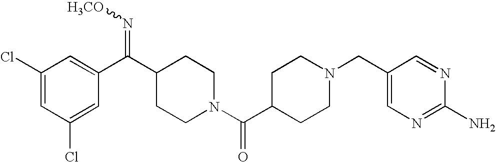 | 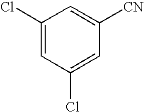 |
| 388 | 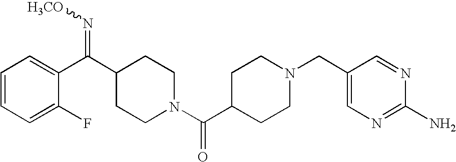 | 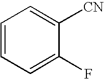 |
| 389 | 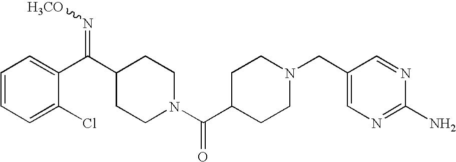 | 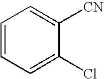 |
| 390 | 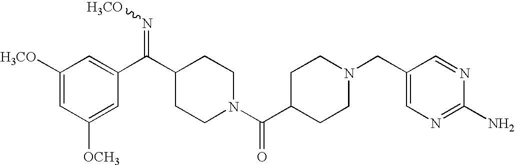 | 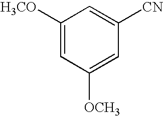 |
| 391 | 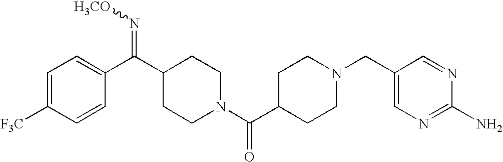 | 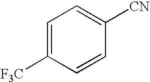 |
| 392 | 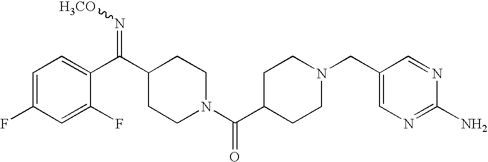 | 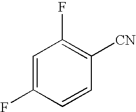 |
| 393 | 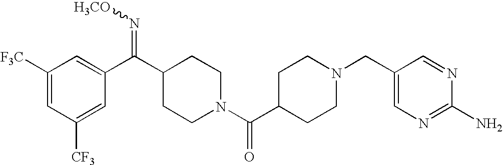 | 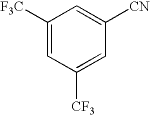 |
| 394 | 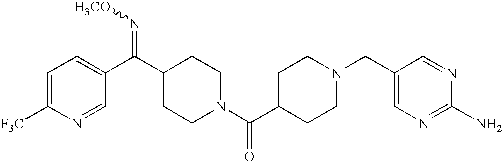 | 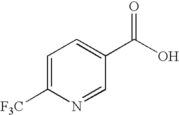 |
| 395 | 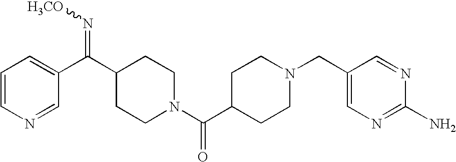 | 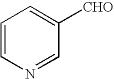 |
| 396 | 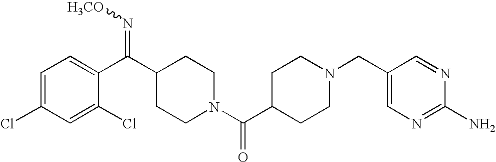 | 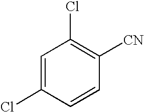 |
| 397 | 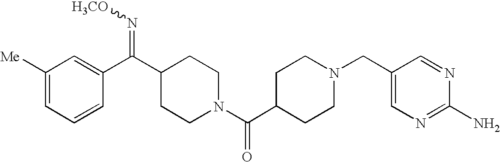 | 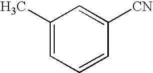 |
| 398 | 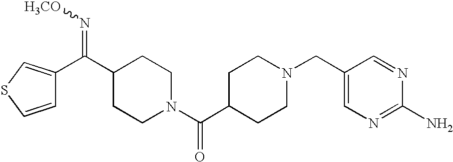 | 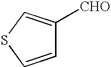 |
| 399 | 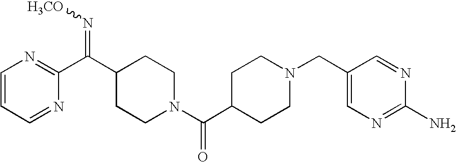 | 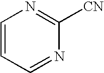 |
| 400 | 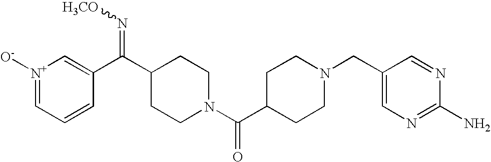 | 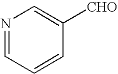 |
| 401 | 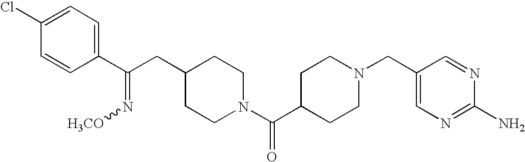 | 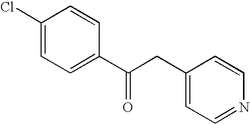 |
| 402 | 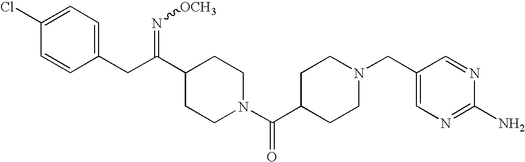 | 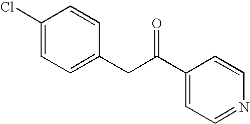 |
| 403 | 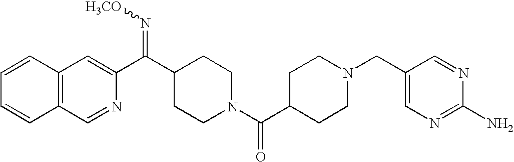 | 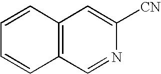 |
| 404 | 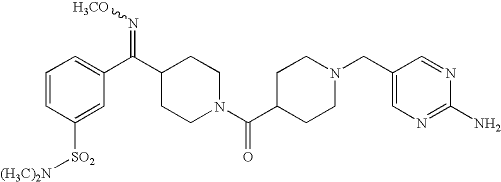 | 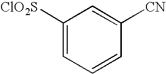 |
| 405 | 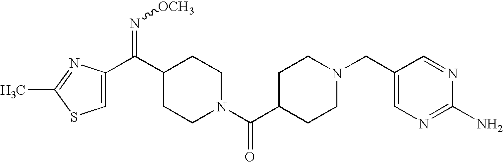 | 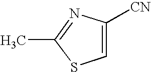 |
| 406 | 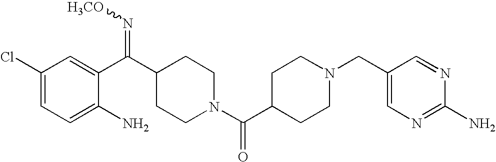 | 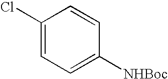 |
| 407 | 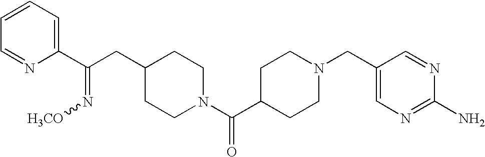 | 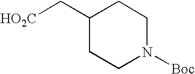 |
| 408 | 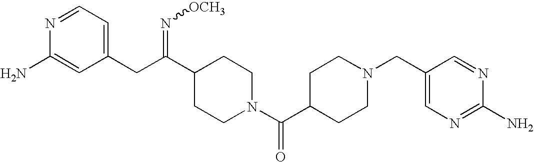 | 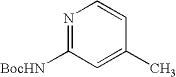 |
| 409 | 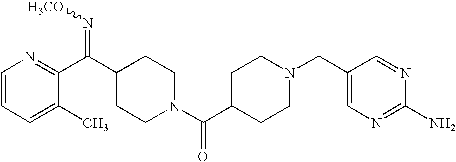 | 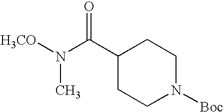 |
| 410 | 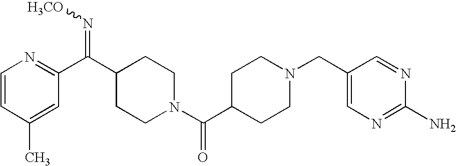 | 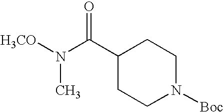 |
| 411 | 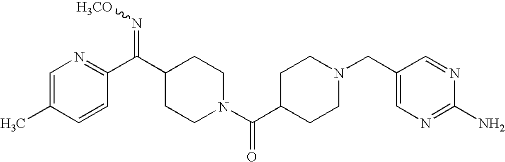 | 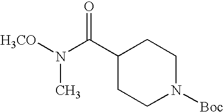 |
| 412 | 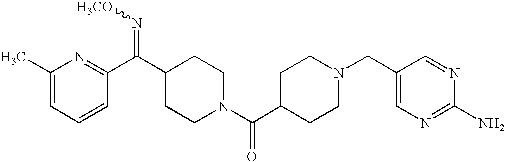 | 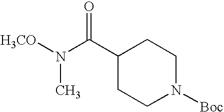 |
| 413 | 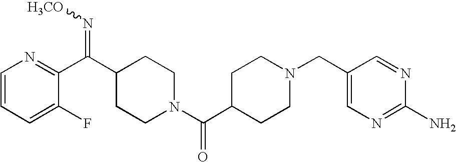 | 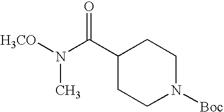 |
| 414 | 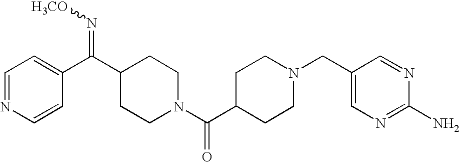 | 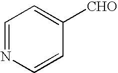 |
| 415 | 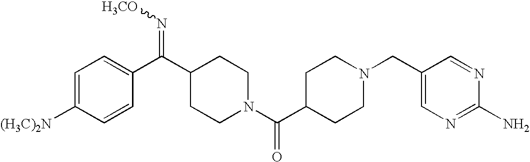 | 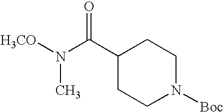 |
| 416 | 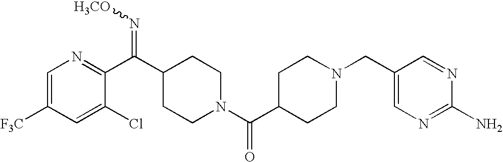 | 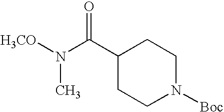 |
| 417 | 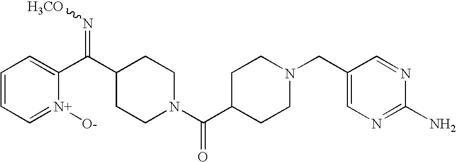 | 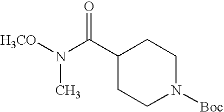 |
| 418 | 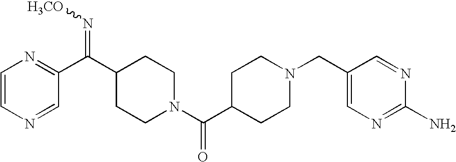 | 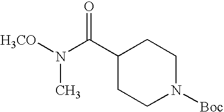 |
| 419 | 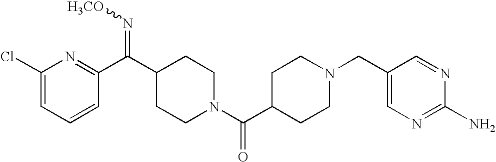 | 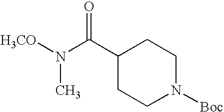 |
| 420 | 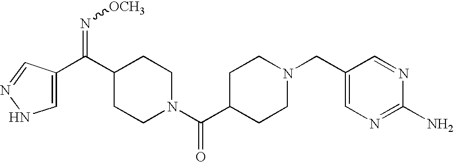 | 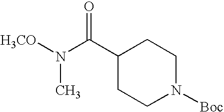 |
| 421 | 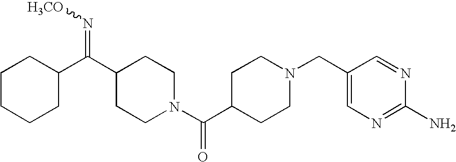 | 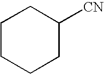 |
| 422 | 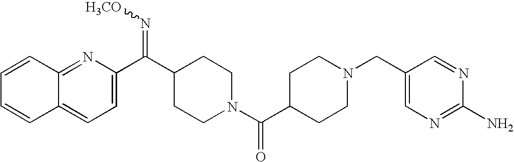 | 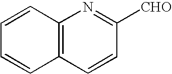 |
| 423 | 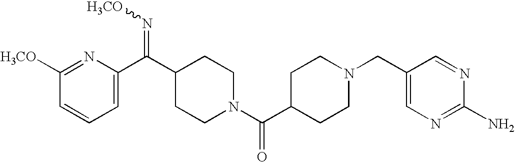 | 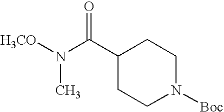 |
| 424 | 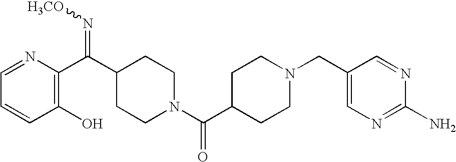 | 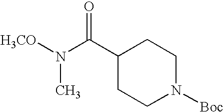 |
| 425 | 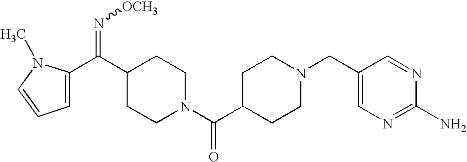 | 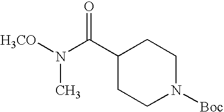 |
| 426 | 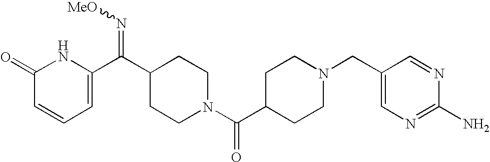 | 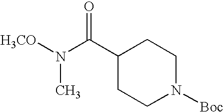 |
| 427 | 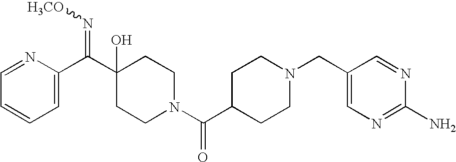 | 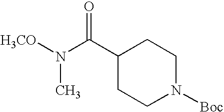 |
| 428 | 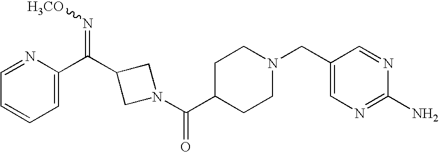 | 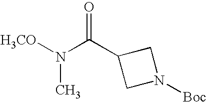 |
| 429 | 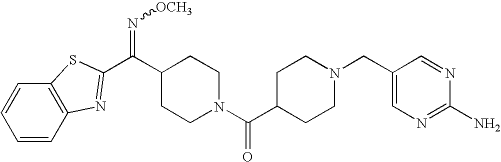 | 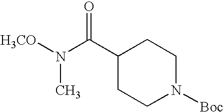 |
| 430 | 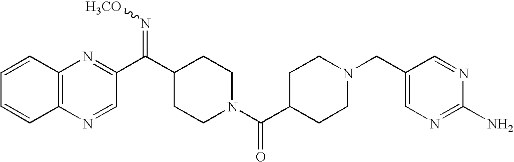 | 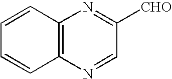 |
| 431 | 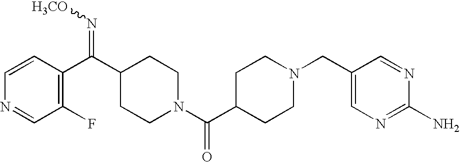 | 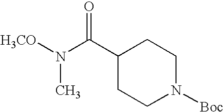 |
| 432 | 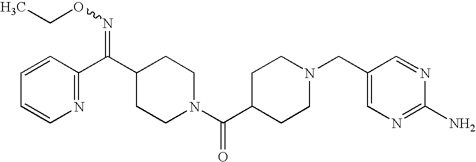 | 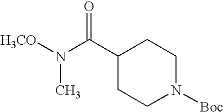 |
| 433 | 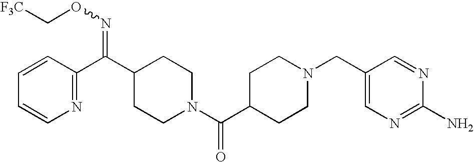 | 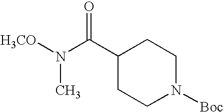 |
| 434 | 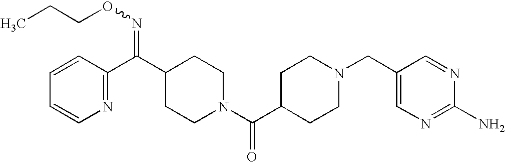 | 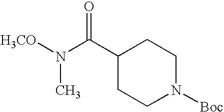 |
| 435 | 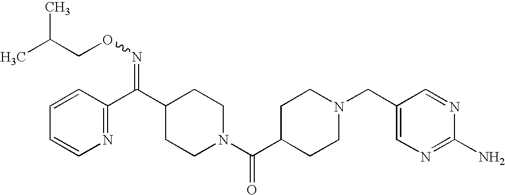 | 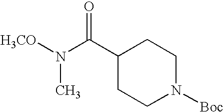 |
| 436 | 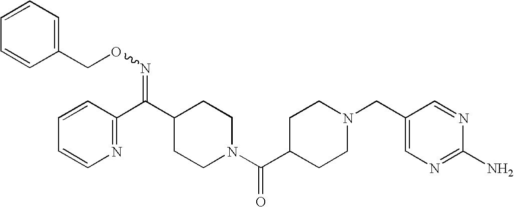 | 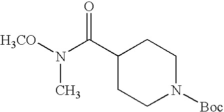 |
| 437 | 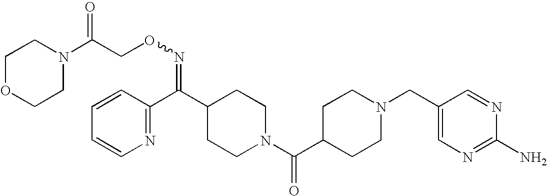 | 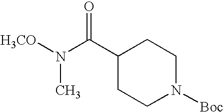 |
| 438 | 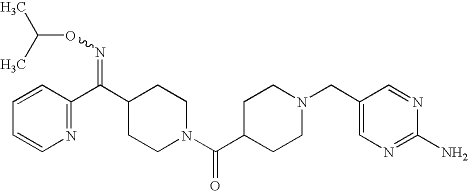 | 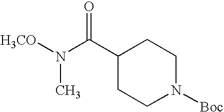 |
| 439 | 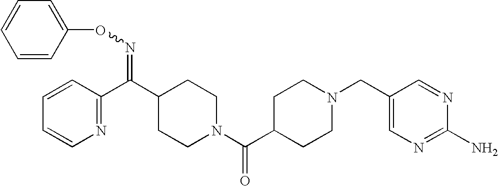 | 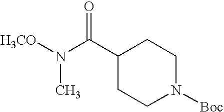 |

### Example 31

### Preparation of Compound 446

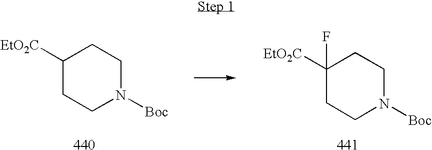

To a solution of LDA (233 mL, 2.0 M in THF/heptane/ethylbenzene, 0.466 mol) in THF (300 mL) at 0° C. was added, dropwise over 1.0 h, a solution of compound 440 (100 g, 0.389 mol) in THF (˜400 mL). The red-orange solution was stirred at 0° C. for 30 min, and then transferred by cannula to a pre-cooled (0° C.) solution of N-fluorobenzenesulfonimide (153 g, 0.485 mol) in dry THF (˜600 mL). The reaction mixture was stirred at 0° C. for 30 min, and then at rt for 18 h. The total solvent volume was reduced to approximately one third, and EtOAc (˜1 L) was added. The solution was washed successively with water, 0.1 N aq. HCl, saturated aq. NaHCO₃, and brine. The organic layer was dried over MgSO₄, filtered, and concentrated under reduced pressure to yield a crude liquid. Separation by flash chromatography (6:1 hexanes-EtOAc) gave compound 441 (93.5 g, 87%).

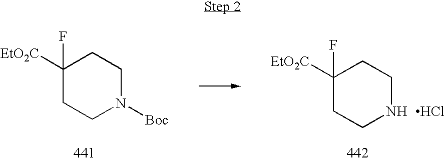

In a manner similar to that described in Example 6, Step 4, compound 441 was converted to compound 442.

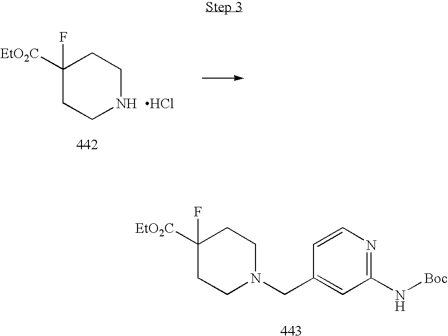

In a manner similar to that described in Example 1, Step 4, compound 442 was converted to compound 443.

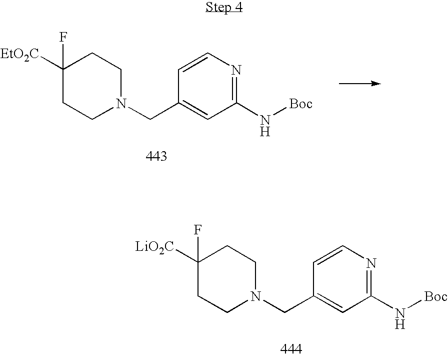

In a manner similar to that described in Example 1, Step 5, compound 443 was converted to compound 444.

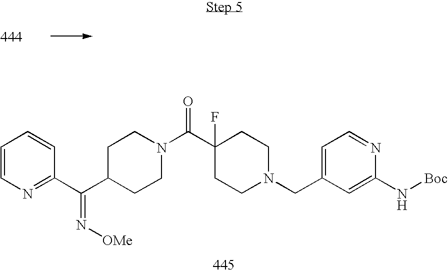

In a manner similar to that described in Example 6, Step 5, compound 444 was converted to compound 445.

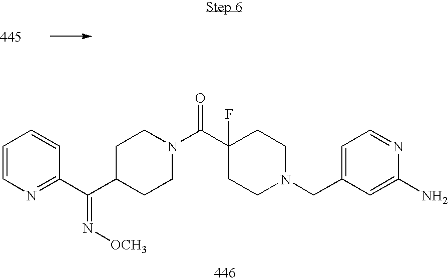

In a manner similar to that described in Example 6, Step 6, compound 445 was converted to compound 446.

In the above examples, the compound 4-[(E)-(methoxyimino)-2-pyridinylmethyl]piperidine dihydrochloride:

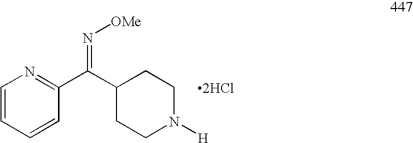

can be used to prepare the compounds of this invention, for example, see Examples 6 and 28. Preferably, Compound 447 is prepared from a compound of formula:

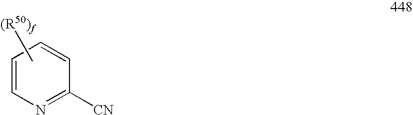

and from a compound of Formula 449:

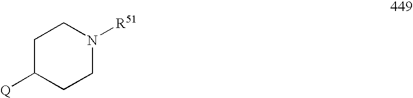

R⁵⁰ is an alkyl or aryl group, f is 0 to 4, R⁵¹ is an alkyl group, and Q is a halo group, wherein said alkyl, aryl, and halo groups are as defined above.

Compound 447 can be prepared from 448 and 449 by:
(a) converting the compound of formula 449 into its Grignard form (449A):

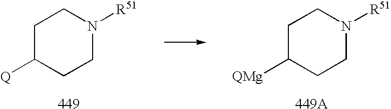

(b) reacting the compound of formula 448 with the compound of formula 449A to obtain a compound of formula 450:

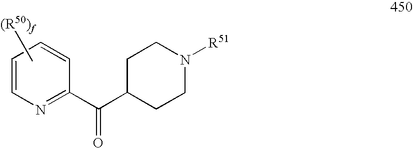

(c) reacting the compound of formula 450 with a suitable alkyl chloroformate of formula 451

R⁵¹—OCOCl 451

to yield a compound of formula 452:

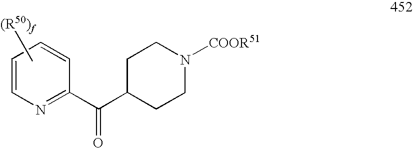

(d) forming the salt (formula 453):

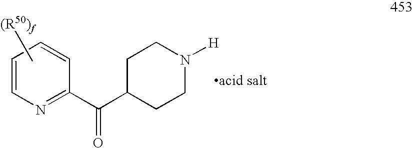

(e) reacting the compound of formula 453 with an alkoxyamine (NH₂OR⁵¹) or its hydrochloride to form an oxime of formula 454:

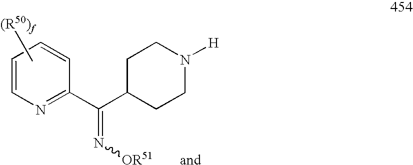

(f) isomerizing the compound of formula 454 by treatment with a strong acid and simultaneously converting to the desired acid salt of Formula 454 with an enriched E isomer, wherein the E isomer predominates over the Z-isomer by at least a 90:10 ratio. When f=0, R⁵¹ is methyl, and the acid used for isomerization is HCl in the compound of formula 454, the final product is the compound of formula 447.

This preparation can be represented as follows:

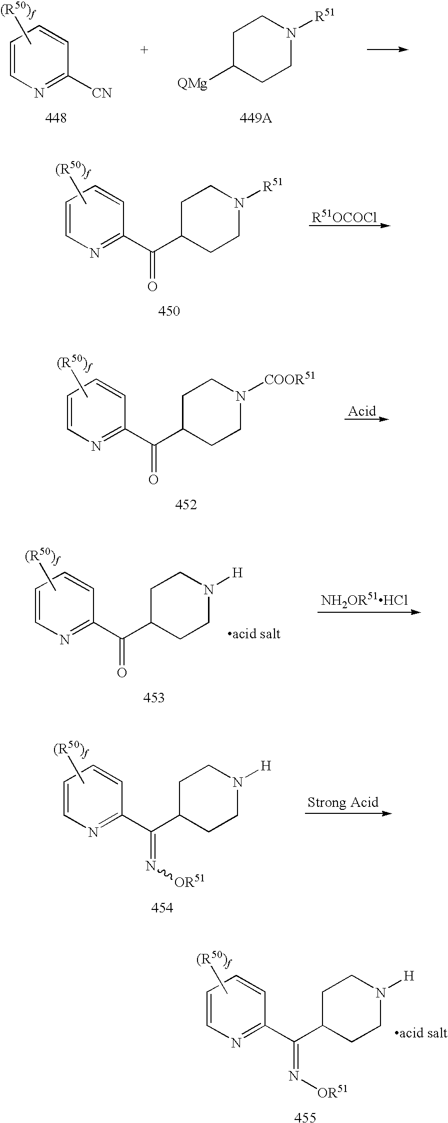

Following the above process the Compound 447 can be prepared as follows:

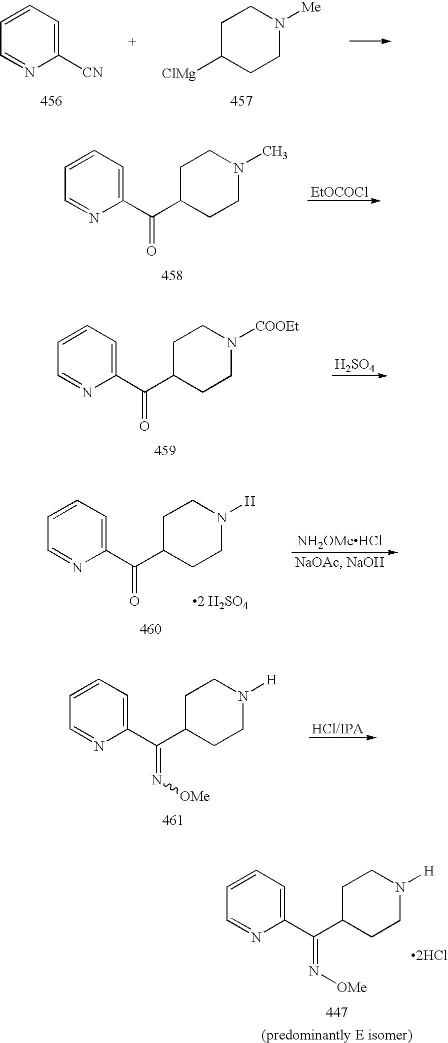

The conversion of compound 461 to 447 predominantly yields the E-isomer of compound 447 in high stereochemical purity and high yields. Isomerization of a mixture of phenyl compounds by acid catalysis is discussed by T. Zsuzsanna et al, Hung. Magy. Km. Foly., 74(3) (1968), 116-119.

The above process starts with Compound 449. In step 1, a 4-halo-1-alkylpiperidine (or a 4-halo-1-arylpiperidine) is converted to its Grignard analog (449A) by reacting with magnesium. The reaction is performed generally at temperatures of about −10° C. to reflux. Generally a hydrocarbon solvent such as, for example, toluene, xylene, chlorobenzene, dichlorobenzene and the like, or mixture of hydrocarbons listed above with an ether, such as, for example, a C₅-C₁₂ alkyl ether, 1,2-dimethoxyethane, 1,2-diethoxyethane, diglyme, 1,4-dioxane, tetrahydrofuran and the like are suitable for this reaction. The solution is cooled to around −10° C. to about 10° C. and then reacted with a suitable 2-cyanopyridine (448), for about 10-120 minutes. Examples of suitable 2-cyanopyridines are 2-cyanopyridine, 4-methyl-2-cyanopyridine, 4-ethyl-2-cyanopyridine, 4-phenyl-2-cyanopyridine, and the like. Preferred are 2-cyanopyridine and 4-methyl-2-cyanopyridine. The Grignard compound is used generally in about 1-4 molar equivalents with respect to the compound of formula 448, preferably in about 1-3 molar equivalents and typically in about 1.5-2.5 molar equivalents. The product of formula 450 may be isolated by procedures well known in the art, such as, for example, treatment with an acid (e.g. HCl), preferably in a suitable solvent (e.g., tetrahydrofuran or ethyl acetate).

The product of Formula 450 may then be reacted with an alkyl chloroformate in the next step. Suitable alkyl chloroformates are, for example, methyl chloroformate, ethyl chloroformate, propyl chloroformate, and the like, with the preferred being methyl chloroformate or ethyl chloroformate. Generally a hydrocarbon solvent such as, for example, toluene, xylene, chlorobenzene, dichlorobenzene and the like, or mixture of a hydrocarbons listed above with an ether such as, for example, a C₅-C₁₂ alkyl ether, 1,2-dimethoxyethane, 1,2-diethoxyethane, diglyme, 1,4-dioxane, tetrahydrofuran and the like is suitable for this reaction. The reaction is generally performed at about 25-100° C., preferably about 40-90° C. and typically about 50-80° C., for about 1-5 hours. After the reaction, generally the generated acid is washed off and the product of formula 452 may be isolated by organic solvent extraction.

The compound of Formula 452 may then be converted into its acid salt by treatment with an acid such as, for example, sulfuric acid, hydrochloric acid, trifluoroacetic acid and the like, generally in a solvent at temperatures between ambient and reflux of the solvent. Suitable solvents include hydrocarbons such as, for example, toluene, xylene, chlorobenzene, dichlorobenzene and the like. There being two nitrogen atoms in the compound of Formula 452, the salt generally has 2 moles of acid to a mole of compound 452.

The compound of formula 453 may then be converted to an alkyloxime of formula 454 by reacting it with an alkoxyamine (or its hydrochloride), usually in aqueous solution form. Suitable alkoxyamines are, for example, methoxyamine, ethoxyamine and the like. Methoxyamine is preferred. The alkoxyamine (or its hydrochloride) is employed generally in about 1 to about 4 molar equivalents, preferably in about 1 to about 3 molar equivalents, and typically in about 1 to about 2 molar equivalents. Generally, the reaction is catalyzed by a weak acid such as, for example, acetic acid, formic acid and the like, or mixtures thereof. A cosolvent such as, for example, methanol, ethanol, isopropanol, n-butanol and the like, or mixtures thereof may be added. The product of formula 454, after work-up, is a mixture of the Z- and the E-isomers, whose ratio may be analyzed for its stereochemical make-up, using techniques well known in the art such as, for example, HPLC.

Treating the compound of formula 454 with a strong acid under the reaction conditions described below isomerizes the mixture of the Z and the E-isomers into predominantly the E-isomer. Generally, the compound of formula 454 may be dissolved in a solvent such as, for example, ethanol, methanol, isopropanol, n-butanol and the like, ether such as methyl tert-butyl ether, tetrahydrofuran and the like, hydrocarbon such as, for example, heptane, hexane, toluene and the like, nitrile such as, for example, acetonitrile, benzonitrile and the like, or mixtures of such solvents. The dissolved compound is then treated with a strong acid such as, for example, HCl, HBr, H₂SO₄ and the like, at temperatures in the range of 20 to 100° C. for about 1-20 hours. The acid is employed generally in about 1 to about 8 molar equivalents, preferably in about 1 to about 6 molar equivalents, and typically in about 2 to about 4 molar equivalents. Work-up typically forms predominantly the acid salt of the E-isomer of the compound of formula 454, which is in fact the compound of formula 447 when R⁵¹=methyl, n=0 and the acid salt is HCl in 454.

The products of the various steps in the process described above may be isolated and purified by conventional techniques such as, for example, filtration, recrystallization, solvent extraction, distillation, precipitation, sublimation and the like, as is well known to those skilled in the art. The products may be analyzed and/or checked for purity by conventional methods such as, for example, thin layer chromatography, NMR, HPLC, melting point, mass spectral analysis, elemental analysis and the like, well known to those skilled in the art.

### Example 32

### Guinea Pig H₃ Receptor Binding Assay

The source of the H₃ receptors in this experiment was guinea pig brain obtained from animals weighing 400-600 g. The brain tissue was homogenized with a solution of 50 mM Tris, pH 7.5. The final concentration of tissue in the homogenization buffer was 10% w/v. The homogenates were centrifuged at 1,000×g for 10 minutes in order to remove clumps of tissue and debris. The resulting supernatants were then centrifuged at 50,000×g for 20 minutes in order to sediment the membranes, which were then washed three times in homogenization buffer (50,000×g for 20 minutes each). The membranes were frozen and stored at −70° C. until needed.

All compounds to be tested were dissolved in DMSO and then diluted into the binding buffer (50 mM Tris, pH 7.5) such that the final concentration was 2 μg/mL with 0.1% DMSO. Membranes were then added (400 μg of protein) to the reaction tubes. The reaction was started by the addition of 3 nM [³H]R-α-methyl histamine (8.8 Ci/mmol) or 3 nM [³H]N^{α}-methyl histamine (80 Ci/mmol) and continued under incubation at 30° C. for 30 minutes. Bound ligand was separated from unbound ligand by filtration, and the amount of radioactive ligand bound to the membranes was quantitated by liquid scintillation spectrometry. All incubations were performed in duplicate and the standard error was always less than 10%. Compounds that inhibited more than 70% of the specific binding of radioactive ligand to the receptor were serially diluted to determine a Kᵢ (nM).

Using this method, the following data were obtained for selected Compounds of Formula (I):

Compounds 44, 45, 49, 75, 76, 83, 88, 99, 104, 110, 117, 128, 200, 201, 203-211, 213, 214, 217, 220-223, 228, 230-232, 234, 236, 239-241, 244-245, 249, 250, 252, 254-267, 274 and 282 had a Kᵢ within the range of from about 0.3 nM to about 370 nM.

Compounds 23, 50, 53, 57A, 59, 92, 212, 215, 218, 219, 220, 224, 226, 227, 229, 233, 235, 238, 246, 247, 248, 251, 253, 268-272, 275, 278, 279, 281 and 287 had a Kᵢ within the range of from about 0.3 nM to about 33 nM.

Compounds 30, 32 31, 33, 54, 55, 56, 56A, 225, 237, 246A, 253A, 273 and 280 had a Kᵢ within the range of from about 0.83 nM to about 16 nM.

### Example 33

### Human H₃ Receptor Binding Assay

The full-length human histamine H₃ receptor was cloned by PCR from a human thalamus cDNA library, with primers derived from a public database, and inserted into the CMV promoter-driven expression vector pcDNA-3.1 (Invitrogen). HEK-293 human embryonic kidney cells (ATCC) were transfected with H₃ receptor plasmid and stably expressing cells were selected with G-418. Cells were grown in Dulbecco's modified Eagle's medium/10% fetal calf serum containing high glucose, 25 mM Hepes, penicillin (100 U/ml), streptomycin (100 ug/ml), 2 mM glutamine, and 0.5 mg G-418/ml at 37° C. in a humidified atmosphere of 5% CO₂.

For membrane preparations, cells were harvested using aspirating media, replacing it with 5 mM EDTA/0.02% trypsin/Hank's balanced salt solution, followed by incubation at 37° C. for 5 to 10 minutes. Cells were decanted and centrifuged at 4° C. for 10 minutes at 1000×g, then resuspended in 50 mM Tris.HCl (ph 7.4) and disrupted for 30 seconds with a Polytron (PT10 tip at setting 6). Homogenates were then centrifuged for ten minutes at 1000×g and the supernatant was decanted and centrifuged for an additional ten minutes at 50,000×g. The pellets obtained were resuspended in Tris buffer and again centrifuged for ten minutes at 50,000×g. Membranes were stored at −80° C. as suspensions of 1 mg of protein/mL of Tris buffer.

For binding assays, membranes were dispersed by Polytron and incubated in 200 mL 50 mM Tris.HCl (pH 7.4) with 1 nM [3H]N-α-methylhistamine and a compound of the invention at concentrations, each in duplicate, equivalent to half orders of magnitude over a five order-of-magnitude range. Nonspecific binding was determined in the presence of 10-5 M thioperamide. After a 30 minute incubation at 30° C., assay mixtures were filtered through 0.3% polyethylenimine-soaked GF/B glass fiber filters, which were then rinsed thrice with buffer, dried, impregnated with Meltilex wax scintillant, and counted. IC₅₀ values were determined from curves fit to the data using a non-linear, least-squares, curve-fitting program and Ki values were determined using the method of Cheng and Prusoff.

Using this method, Compound 287 was determined to have a Ki value of 25±4 nM (n=4).

### Example 34

### In Vivo Effect of Compound 446 on Glucose Levels in Diabetic Mice

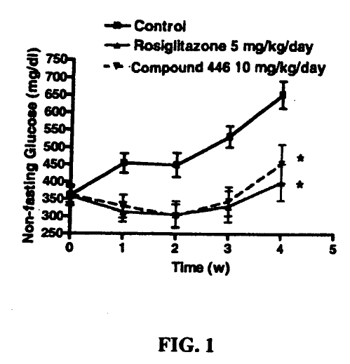

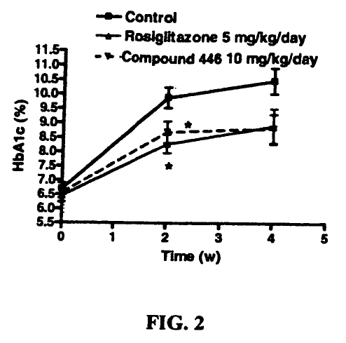

Five-week-old male ICR mice were purchased from Taconic Farm (Germantown, N.Y.) and placed on a “western diet” containing 45% (kcal) fat from lard and 0.12% (w/w) cholesterol. After 3 weeks of feeding, the mice were injected once with low dose streptozocin (STZ, ip 80 mg/kg) to induce partial insulin deficiency. Two weeks after receiving the STZ injection, the majority of the STZ-treated mice developed type 2 diabetes and displayed hyperglycemia, insulin resistance, and glucose intolerance. The diabetic mice were then placed in one of three groups: (1) a non-treated control group, (2) a group treated with rosiglitazone (5 mg/kg/day in diet); or (3) a group treated with Compound 446 (10/mg/kg/day in diet). All animals were treated for four weeks. As illustrated in FIGS. 1 and 2, mice treated with Compound 446 (10 mg/kg/day in diet) had significantly reduced non-fasting glucose and HbA1C levels relative to control mice and mice treated with rosiglitazone (5 mg/kg/day in diet).

Accordingly, Compound 446, an illustrative Compound of Formula (I), is effective for treating diabetes in a patient.

### Example 35

### In Vivo Effect of Compound 446 on Glucose Levels in Diabetic Rats

Adult, diabetic, Goto-Kakizaki rats (14 weeks old) were tested for non-fasting glucose levels using a glucometer. Rats with glucose levels between 130 and 370 mg/dl were randomized into treatment (N=10) and control (N=10) groups. Animals in the treatment group were administered Compound 446 in their food chow at a dose of 10 mg/kg/day. After one week of treatment, blood was collected via tail snip and the non-fasting glucose level was measured using a glucometer.

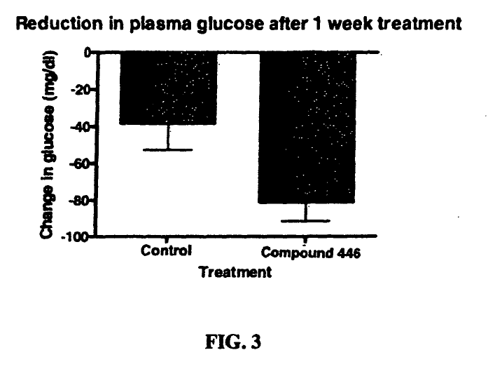

As illustrated in FIG. 3, rats treated with compound 446 had an average reduction in non-fasting glucose levels of 81 mg/dl, compared to an average a reduction in non-fasting glucose levels of 34 mg/dl for untreated rats.

Accordingly, Compound 446, an illustrative Compound of Formula (I), is effective for treating diabetes in a patient.

### Example 36

### In Vivo Effect of Compound 287 on Glucose Levels in Diabetic Rats

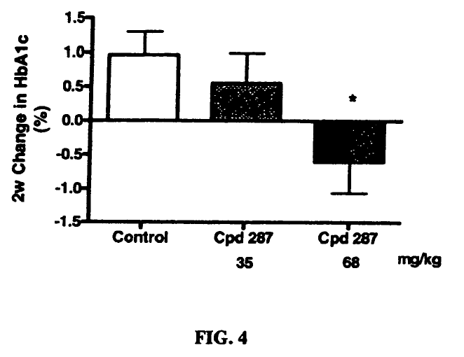

Seventy male DIO Sprague-Dawley rats were fed HFD (45% Kcal fat) for 3 months from weaning, and were given streptozotocin (STZ) intraperitoneally at 25 mg/kg to induce type 2 diabetes (T2DM). Forty four T2DM rats were chosen for the study two weeks after STZ injection (n=11 per group, with body weights between 632 and 838 g, non-fasting glucose between 226 and 426 mg/dl and HbA1c between 8.7% and 10.9%) and were given ad libitum access to pre-weighed 45% fat (kcal) HFD or Compound 287 (1.4, 2.9 mg/g in HFD) for two weeks. Body weight, non-fasting glucose and food intake were monitored daily. Body composition and HbA1c levels were monitored before and after the two-week study by the whole body magnetic resonance analyzer and Cholestech GDX analyzer (Hayward, Calif.), respectively. The STZ-DIO rats had elevated non-fasting glucose and HbA1c levels (non-fasting glucose were between 226 and 426 mg/dl; and HbA1c were between 8.7% and 10.9%) two weeks after STZ injection. The low dose of STZ caused a 48% reduction of plasma insulin levels, which was not sufficient to cause hyperglycemia in rats fed with chow diet. In contrast, this level of plasma insulin induced hyperglycemia in the face of insulin resistance induced by the HFD. As illustrated in FIG. 4, Compound 287 caused a dose-dependent reduction of HbA1c levels over the two week study period. The control STZ-DIO rats maintained non-fasting glucose levels above 350 mg/ml (+12 mg/dl), which led to a significant 0.96% increase in HbA1c over 14 days. STZ-DIO rats treated with Compound 287 (68 mg/kg/day, 2.9 mg/g in HFD) had significantly reduced non-fasting glucose (−43 mg/dl) which led to a 0.6% decrease in HbA1c level in two weeks.

Accordingly, Compound 287, an illustrative Compound of Formula (I) is effective for treating diabetes in a patient.

### Methods of Using the Compounds of Formula (I)

The Compounds of Formula (I) are useful for treating or preventing a Condition a patient.

### Methods for Treating or Preventing Pain

The Compounds of Formula (I) are useful for treating or preventing pain in a patient.

Accordingly, in one embodiment, the present invention provides a method for treating pain in a patient, comprising administering to the patient an effective amount of one or more Compounds of Formula (I).

Illustrative examples of pain treatable or preventable using the present methods, include, but are not limited to acute pain, chronic pain, neuropathic pain, nociceptive pain, cutaneous pain, somatic pain, visceral pain, phantom limb pain, diabetic pain, cancer pain (including breakthrough pain), pain caused by drug therapy (such as cancer chemotherapy), headache (including migraine, tension headache, cluster headache, pain caused by arithritis, pain caused by injury, toothache, or pain caused by a medical procedure (such as surgery, physical therapy or radiation therapy).

In one embodiment, the pain is neuropathic pain.

In another embodiment, the pain is cancer pain.

In another embodiment, the pain is headache.

In still another embodiment, the pain is chronic pain.

In a further embodiment, the pain is diabetic pain.

### Methods for Treating or Preventing Diabetes

The Compounds of Formula (I) are useful for treating or preventing diabetes in a patient. Accordingly, in one embodiment, the present invention provides a method for treating diabetes in a patient, comprising administering to the patient an effective amount of one or more Compounds of Formula (I).

Examples of diabetes treatable or preventable using the Compounds of Formula (I) include, but are not limited to, type I diabetes (insulin-dependent diabetes mellitus), type II diabetes (non-insulin dependent diabetes mellitus), gestational diabetes, diabetes caused by administration of anti-psychotic agents, diabetes caused by administration of anti-depressant agents, diabetes caused by administration of steroid drugs, autoimmune diabetes, insulinopathies, diabetes due to pancreatic disease, diabetes associated with other endocrine diseases (such as Cushing's Syndrome, acromegaly, pheochromocytoma, glucagonoma, primary aldosteronism or somatostatinoma), type A insulin resistance syndrome, type B insulin resistance syndrome, lipatrophic diabetes, diabetes induced by β-cell toxins, and diabetes induced by drug therapy (such as diabetes induced by antipsychotic agents).

In one embodiment, the diabetes is type I diabetes.

In another embodiment, the diabetes is type II diabetes.

In another embodiment, the diabetes is gestational diabetes.

### Methods for Treating or Preventing a Diabetic Complication

The Compounds of Formula (I) are useful for treating or preventing a diabetic complication in a patient. Accordingly, in one embodiment, the present invention provides a method for treating a diabetic complication in a patient, comprising administering to the patient an effective amount of one or more Compounds of Formula (I).

Examples of diabetic complications treatable or preventable using the Compounds of Formula (I) include, but are not limited to, diabetic cataract, glaucoma, retinopathy, aneuropathy (such as diabetic neuropathy, polyneuropathy, mononeuropathy, autonomic neuropathy, microaluminuria and progressive diabetic neuropathyl), nephropathy, diabetic pain, gangrene of the feet, immune-complex vasculitis, systemic lupsus erythematosus (SLE), atherosclerotic coronary arterial disease, peripheral arterial disease, nonketotic hyperglycemic-hyperosmolar coma, foot ulcers, joint problems, a skin or mucous membrane complication (such as an infection, a shin spot, a candidal infection or necrobiosis lipoidica diabeticorumobesity), hyperlipidemia, hypertension, syndrome of insulin resistance, coronary artery disease, a fungal infection, a bacterial infection, and cardiomyopathy.

In one embodiment, the diabetic complication is neuropathy.

In another embodiment, the diabetic complication is retinopathy.

In another embodiment, the diabetic complication is nephropathy.

### Methods for Treating or Preventing Impaired Glucose Tolerance

The Compounds of Formula (I) are useful for treating or preventing impaired glucose tolerance in a patient.

Accordingly, in one embodiment, the present invention provides a method for treating impaired glucose tolerance in a patient, comprising administering to the patient an effective amount of one or more Compounds of Formula (I).

### Methods for Treating or Preventing Impaired Fasting Glucose

The Compounds of Formula (I) are useful for treating or preventing impaired fasting glucose in a patient.

Accordingly, in one embodiment, the present invention provides a method for treating impaired fasting glucose in a patient, comprising administering to the patient an effective amount of one or more Compounds of Formula (I).

### Combination Therapy

In one embodiment, the present invention provides methods for treating a Condition in a patient, the method comprising administering to the patient one or more Compounds of Formula (I), or a pharmaceutically acceptable salt, solvate, ester or prodrug thereof and at least one additional therapeutic agent that is not a Compound of Formula (I), wherein the amounts administered are together effective to treat or prevent a Condition.

When administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like. The amounts of the various actives in such combination therapy may be different amounts (different dosage amounts) or same amounts (same dosage amounts).

In one embodiment, the one or more Compounds of Formula (I) is administered during at time when the additional therapeutic agent(s) exert their prophylactic or therapeutic effect, or vice versa.

In another embodiment, the one or more Compounds of Formula (I) and the additional therapeutic agent(s) are administered in doses commonly employed when such agents are used as monotherapy for treating a Condition.

In another embodiment, the one or more Compounds of Formula (I) and the additional therapeutic agent(s) are administered in doses lower than the doses commonly employed when such agents are used as monotherapy for treating a Condition.

In still another embodiment, the one or more Compounds of Formula (I) and the additional therapeutic agent(s) act synergistically and are administered in doses lower than the doses commonly employed when such agents are used as monotherapy for treating a Condition.

In one embodiment, the one or more Compounds of Formula (I) and the additional therapeutic agent(s) are present in the same composition. In one embodiment, this composition is suitable for oral administration. In another embodiment, this composition is suitable for intravenous administration.

The one or more Compounds of Formula (I) and the additional therapeutic agent(s) can act additively or synergistically. A synergistic combination may allow the use of lower dosages of one or more agents and/or less frequent administration of one or more agents of a combination therapy. A lower dosage or less frequent administration of one or more agents may lower toxicity of the therapy without reducing the efficacy of the therapy.

In one embodiment, the administration of one or more Compounds of Formula (I) and the additional therapeutic agent(s) may inhibit the resistance of a Condition to these agents.

In one embodiment, when the patient is treated for diabetes, a diabetic complication, impaired glucose tolerance or impaired fasting glucose, the other therapeutic is an antidiabetic agent which is not a Compound of Formula (I). In another embodiment, when the patient is treated for pain, the other therapeutic agent is an analgesic agent which is not a Compound of Formula (I).

In another embodiment, the other therapeutic agent is an agent useful for reducing any potential side effect of a Compound of Formula (I). Such potential side effects include, but are not limited to, nausea, vomiting, headache, fever, lethargy, muscle aches, diarrhea, general pain, and pain at an injection site.

In one embodiment, the other therapeutic agent is used at its known therapeutically effective dose. In another embodiment, the other therapeutic agent is used at its normally prescribed dosage. In another embodiment, the other therapeutic agent is used at less than its normally prescribed dosage or its known therapeutically effective dose.

Examples of antidiabetic agents useful in the present methods for treating diabetes or a diabetic complication include a sulfonylurea; an insulin sensitizer; a glucosidase inhibitor; an insulin secretagogue; a hepatic glucose output lowering agent; an anti-obesity agent; an antihypertensive agent; a meglitinide; an agent that slows or blocks the breakdown of starches and sugars in vivo; an histamine H₃ receptor antagonist; an antihypertensive agent, a sodium glucose uptake transporter 2 (SGLT-2) inhibitor; a peptide that increases insulin production; and insulin or any insulin-containing composition.

In one embodiment, the antidiabetic agent is an insulin sensitizer.

Non-limiting examples of insulin sensitizers include PPAR activators, such as the glitazone and thiazoldinedione class of agents, which include rosiglitazone, rosiglitazone maleate (AVANDIA™ from GlaxoSmithKline), pioglitazone, pioglitazone hydrochloride (ACTOS™, from Takeda) ciglitazone and MCC-555 (Mitstubishi Chemical Co.), troglitazone and englitazone; biguanides, such as phenformin, metformin, metformin hydrochloride (such as GLUCOPHAGE® from Bristol-Myers Squibb), metformin hydrochloride with glyburide (such as GLUCOVANCE™ from Bristol-Myers Squibb) and buformin; DPP-IV inhibitors, such as sitagliptin, saxagliptin (Januvia™, Merck), denagliptin, vildagliptin (Galvus™, Novartis), alogliptin, alogliptin benzoate, ABT-279 and ABT-341 (Abbott), ALS-2-0426 (Alantos), ARI-2243 (Arisaph), BI-A and BI-B (Boehringer Ingelheim), SYR-322 (Takeda), MP-513 (Mitsubishi), DP-893 (Pfizer), RO-0730699 (Roche) or a combination of sitagliptin/metformin HCl (Janumet™, Merck); PTP-1B inhibitors; and α-glucokinase activators, such as miglitol, acarbose, and voglibose.

In one embodiment, the antidiabetic agent is a DPP-IV inhibitor.

In another embodiment, the antidiabetic agent is a sulfonylurea.

Non-limiting examples of sulfonylureas include glipizide, tolbutamide, glyburide, glimepiride, chlorpropamide, acetohexamide, gliamilide, gliclazide, glibenclamide and tolazamide.

In one embodiment, the antidiabetic agent is a SGLT-2 inhibitor.

Non-limiting examples of SGLT-2 inhibitors useful in the present methods include dapagliflozin and sergliflozin, AVE2268 (Sanofi-Aventis) and T-1095 (Tanabe Seiyaku).

In another embodiment, the antidiabetic agent is a hepatic glucose output lowering agent.

Non-limiting examples of hepatic glucose output lowering agents include Glucophage and Glucophage XR.

In another embodiment, the antidiabetic agent is a of histamine H₃ receptor antagonist.

Non-limiting examples of histamine H₃ receptor antagonist agents include the following compound:

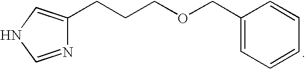

In one embodiment, the antidiabetic agent is an insulin secretagogue.

Non-limiting examples of insulin secretagogues include GLP-1, GLP-1 mimetics, exendin, GIP, secretin, glipizide, chlorpropamide, nateglinide, meglitinide, glibenclamide, repaglinide and glimepiride.

Non-limiting examples of GLP-1 mimetics useful in the present methods include Byetta-Exanatide, Liraglutinide, CJC-1131 (ConjuChem, Exanatide-LAR (Amylin), BIM-51077 (Ipsen/LaRoche), ZP-10 (Zealand Pharmaceuticals), and compounds disclosed in International Publication No. WO 00/07617.

In another embodiment, the antidiabetic agent is insulin or an insulin-containing preparation.

The term “insulin” as used herein, includes all formulations of insulin, including long acting and short acting forms of insulin.

Non-limiting examples of orally administrable insulin and insulin containing compositions include AL-401 from AutoImmune, and the compositions disclosed in U.S. Pat. Nos. 4,579,730; 4,849,405; 4,963,526; 5,642,868; 5,763,396; 5,824,638; 5,843,866; 6,153,632; 6,191,105; and International Publication No. WO 85/05029, each of which is incorporated herein by reference.

In one embodiment, the antidiabetic agent is anti-obesity agent.

Non-limiting examples of anti-obesity agents useful in the present methods for treating diabetes include a 5-HT2C agonist, such as lorcaserin; a neuropeptide Y antagonist; an MCR4 agonist; an MCH receptor antagonist; a protein hormone, such as leptin or adiponectin; an AMP kinase activator; and a lipase inhibitor, such as orlistat. Appetite suppressants are not considered to be within the scope of the anti-obesity agents useful in the present methods.

In another embodiment, the antidiabetic agent is an antihypertensive agent.

Non-limiting examples of antihypertensive agents useful in the present methods for treating diabetes include β-blockers and calcium channel blockers (for example diltiazem, verapamil, nifedipine, amlopidine, and mybefradil), ACE inhibitors (for example captopril, lisinopril, enalapril, spirapril, ceranopril, zefenopril, fosinopril, cilazopril, and quinapril), AT-1 receptor antagonists (for example losartan, irbesartan, and valsartan), renin inhibitors and endothelin receptor antagonists (for example sitaxsentan).

In another embodiment, the antidiabetic agent is a meglitinide.

Non-limiting examples of meglitinides useful in the present methods for treating diabetes include repaglinide and nateglinide.

In still another embodiment, the antidiabetic agent is an agent that slows or blocks the breakdown of starches and sugars in vivo.

Non-limiting examples of antidiabetic agents that slow or block the breakdown of starches and sugars in vivo and are suitable for use in the compositions and methods of the present invention include alpha-glucosidase inhibitors and certain peptides for increasing insulin production. Alpha-glucosidase inhibitors help the body to lower blood sugar by delaying the digestion of ingested carbohydrates, thereby resulting in a smaller rise in blood glucose concentration following meals. Non-limiting examples of suitable alpha-glucosidase inhibitors include acarbose; miglitol; camiglibose; certain polyamines as disclosed in WO 01/47528 (incorporated herein by reference); voglibose. Non-limiting examples of suitable peptides for increasing insulin production including amlintide (CAS Reg. No. 122384-88-7 from Amylin; pramlintide, exendin, certain compounds having Glucagon-like peptide-1 (GLP-1) agonistic activity as disclosed in WO 00/07617 (incorporated herein by reference).

Non-limiting examples of other analgesic agents useful in the present methods for treating pain include acetaminophen, an NSAID, an opiate or a tricyclic antidepressant.

In one embodiment, the other analgesic agent is acetaminophen or an NSAID.

In another embodiment, the other analgesic agent is an opiate.

In another embodiment, the other analgesic agent is a tricyclic antidepressant.

Non-limiting examples of NSAIDS useful in the present methods for treating pain include a salicylate, such as aspirin, amoxiprin, benorilate or diflunisal; an arylalkanoic acid, such as diclofenac, etodolac, indometacin, ketorolac, nabumetone, sulindac or tolmetin; a 2-arylpropionic acid (a “profen”), such as ibuprofen, carprofen, fenoprofen, flurbiprofen, loxoprofen, naproxen, tiaprofenic acid or suprofen; a fenamic acid, such as mefenamic acid or meclofenamic acid; a pyrazolidine derivative, such as phenylbutazone, azapropazone, metamizole or oxyphenbutazone; a coxib, such as celecoxib, etoricoxib, lumiracoxib or parecoxib; an oxicam, such as piroxicam, lornoxicam, meloxicam or tenoxicam; or a sulfonanilide, such as nimesulide.

Non-limiting examples of opiates useful in the present methods for treating pain include an anilidopiperidine, a phenylpiperidine, a diphenylpropylamine derivative, a benzomorphane derivative, an oripavine derivative and a morphinane derivative. Additional illustrative examples of opiates include morphine, diamorphine, heroin, buprenorphine, dipipanone, pethidine, dextromoramide, alfentanil, fentanyl, remifentanil, methadone, codeine, dihydrocodeine, tramadol, pentazocine, vicodin, oxycodone, hydrocodone, percocet, percodan, norco, dilaudid, darvocet or lorcet.

Non-limiting examples of tricyclic antidepressants useful in the present methods for treating pain include amitryptyline, carbamazepine, gabapentin or pregabalin.

The doses and dosage regimen of the other agents used in the combination therapies of the present invention for the treatment or prevention of a Condition can be determined by the attending clinician, taking into consideration the the approved doses and dosage regimen in the package insert; the age, sex and general health of the patient; and the type and severity of the viral infection or related disease or disorder. When administered in combination, the Compound(s) of Formula (I) and the other agent(s) for treating diseases or conditions listed above can be administered simultaneously or sequentially. This is particularly useful when the components of the combination are given on different dosing schedules, e.g., one component is administered once daily and another every six hours, or when the preferred pharmaceutical compositions are different, e.g. one is a tablet and one is a capsule. A kit comprising the separate dosage forms is therefore advantageous.

Generally, a total daily dosage of the one or more Compounds of Formula (I) and the additional therapeutic agent(s)can when administered as combination therapy, range from about 0.1 to about 2000 mg per day, although variations will necessarily occur depending on the target of the therapy, the patient and the route of administration. In one embodiment, the dosage is from about 0.2 to about 100 mg/day, administered in a single dose or in 2-4 divided doses. In another embodiment, the dosage is from about 1 to about 500 mg/day, administered in a single dose or in 2-4 divided doses. In another embodiment, the dosage is from about 1 to about 200 mg/day, administered in a single dose or in 2-4 divided doses. In still another embodiment, the dosage is from about 1 to about 100 mg/day, administered in a single dose or in 2-4 divided doses. In yet another embodiment, the dosage is from about 1 to about 50 mg/day, administered in a single dose or in 2-4 divided doses. In a further embodiment, the dosage is from about 1 to about 20 mg/day, administered in a single dose or in 2-4 divided doses.

### Compositions and Administration

In one embodiment, the invention provides compositions comprising an effective amount of one or more Compounds of Formula (I) or a pharmaceutically acceptable salt, solvate, ester or prodrug thereof, and a pharmaceutically acceptable carrier.

For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g. magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18th Edition, (1990), Mack Publishing Co., Easton, Pa.

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations which are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

In one embodiment, the Compound of Formula (I) is administered orally.

In another embodiment, the Compound of Formula (I) is administered parenterally.

In another embodiment, the Compound of Formula (I) is administered intravenously.

In one embodiment, the pharmaceutical preparation is in a unit dosage form. In such form, the preparation is subdivided into suitably sized unit doses containing appropriate quantities of the active component, e.g., an effective amount to achieve the desired purpose.

The quantity of active compound in a unit dose of preparation is from about 0.1 to about 2000 mg. Variations will necessarily occur depending on the target of the therapy, the patient and the route of administration. In one embodiment, the unit dose dosage is from about 0.2 to about 1000 mg. In another embodiment, the unit dose dosage is from about 1 to about 500 mg. In another embodiment, the unit dose dosage is from about 1 to about 100 mg/day. In still another embodiment, the unit dose dosage is from about 1 to about 50 mg. In yet another embodiment, the unit dose dosage is from about 1 to about 10 mg.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

The amount and frequency of administration of the compounds of the invention and/or the pharmaceutically acceptable salts thereof will be regulated according to the judgment of the attending clinician considering such factors as age, condition and size of the patient as well as severity of the symptoms being treated. A typical recommended daily dosage regimen for oral administration can range from about 1 mg/day to about 300 mg/day, preferably 1 mg/day to 75 mg/day, in two to four divided doses.

When the invention comprises a combination of at least one Compound of Formula (I) and an additional therapeutic agent, the two active components may be co-administered simultaneously or sequentially, or a single pharmaceutical composition comprising at least one Compound of Formula (I) and an additional therapeutic agent in a pharmaceutically acceptable carrier can be administered. The components of the combination can be administered individually or together in any conventional dosage form such as capsule, tablet, powder, cachet, suspension, solution, suppository, nasal spray, etc. The dosage of the additional therapeutic agent can be determined from published material, and may range from about 1 to about 1000 mg per dose. In one embodiment, when used in combination, the dosage levels of the individual components are lower than the recommended individual dosages because of the advantageous effect of the combination.

In one embodiment, the components of a combination therapy regime are to be administered simultaneously, they can be administered in a single composition with a pharmaceutically acceptable carrier.

In another embodiment, when the components of a combination therapy regime are to be administered separately or sequentially, they can be administered in separate compositions, each containing a pharmaceutically acceptable carrier.

The components of the combination therapy can be administered individually or together in any conventional dosage form such as capsule, tablet, powder, cachet, suspension, solution, suppository, nasal spray, etc.

### Kits

In one aspect, the present invention provides a kit comprising a effective amount of one or more Compounds of Formula (I), or a pharmaceutically acceptable salt or solvate of the compound and a pharmaceutically acceptable carrier, vehicle or diluent.

In another aspect the present invention provides a kit comprising an amount of one or more Compounds of Formula (I), or a pharmaceutically acceptable salt or solvate of the compound and an amount of at least one additional therapeutic agent listed above, wherein the combined amounts are effective for treating or preventing a Condition in a patient.

When the components of a combination therapy regime are to are to be administered in more than one composition, they can be provided in a kit comprising in a single package, one container comprising a Compound of Formula (I) in pharmaceutically acceptable carrier, and one or more separate containers, each comprising one or more additional therapeutic agents in a pharmaceutically acceptable carrier, with the active components of each composition being present in amounts such that the combination is therapeutically effective.

The present invention is not to be limited by the specific embodiments disclosed in the examples that are intended as illustrations of a few aspects of the invention and any embodiments that are functionally equivalent are within the scope of this invention. Indeed, various modifications of the invention in addition to those shown and described herein will become apparant to those skilled in the art and are intended to fall within the scope of the appended claims.

A number of references have been cited herein, the entire disclosures of which are incorporated herein by reference.

What is claimed is:

1. A method for treating a condition in a patient, comprising administering to the patient an effective amount of one or more compounds having the formula: 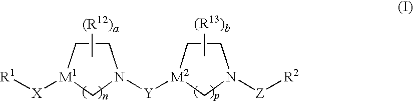
or a pharmaceutically acceptable salt, solvate, ester or prodrug thereof, wherein:
R¹ is aryl, heteroaryl, heterocycloalkyl, alkyl, cycloalkyl or alkylaryl, each of which can be optionally substituted with from 1 to 4 substituents, which are the same or different, and are independently selected from halo, —OH, —O-alkyl, haloalkyl, —OCF₃, —NR⁴R⁵, phenyl, —NO₂, —CO₂R⁴, —CON(R⁴)₂, —S(O)ₘN(R²⁰)₂ and —CN, or R¹ and X are taken together to form: 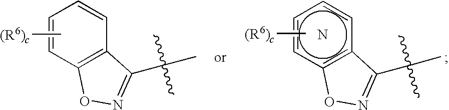
X is —C(O)—, —C(NOR³)—, —C(NNR⁴R⁵)—, 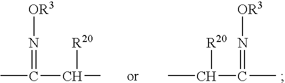
R² is a five or six-membered heteroaryl group, wherein a six-membered heteroaryl group contains 1 or 2 nitrogen ring atoms with the remaining ring atoms being carbon, and a five-membered heteroaryl group contains 1 or 2 hetero ring atoms selected from nitrogen, oxygen, and sulfur, with the remaining ring atoms being carbon; and wherein a five or six membered heteroaryl group can be optionally substituted with from 1 to 3 substituents, which are the same or different, and are independently selected from halo, —OH, alkyl, —O-alkyl, haloalkyl, —OCF₃, —NR⁴R⁵, phenyl, —NO₂, —CO₂R⁴, —CON(R⁴)₂, —CH₂NR⁴R⁵, —(N)C(NR⁴R⁵)₂, and —CN;
R³ is hydrogen, alkyl, aryl, heteroaryl, heterocycloalkyl, arylalkyl, haloalkyl, —CH₂CF_{3,} —(CH₂)ₑ—C(O)N(R⁴)₂, —(CH₂)ₑ—C(O)OR⁴ or —(CH₂)ₑ—C(O)R³⁰, wherein an aryl, heteroaryl or heterocycloalkyl group, or the aryl portion of an arylalkyl group can be optionally substituted with from 1 to 3 substituents, which are the same or different, and are independently selected from halo, —OH, —OCF₃, haloalkyl, —CN, —N(R⁴⁵)₂, —CO₂R⁴⁵ and —C(O)N(R⁴⁵)₂;
each occurrence of R⁴ is independently hydrogen, alkyl, aryl or alkylaryl, wherein an aryl group or the aryl moiety of an alkylaryl group can be optionally substituted with 1 to 3 substituents, which are the same or different, and are independently selected from halo, haloalkyl, —OCF₃, —OH, —N(R⁴⁵)₂, —CO₂R⁴⁵, —C(O)N(R⁴⁵)₂ and —CN;
R⁵ is hydrogen, alkyl, —C(O)R⁴, —C(O)₂R⁴ or —C(O)N(R⁴)₂, or R⁴ and R⁵ taken together with the nitrogen atom to which they are both attached, join to form a five- or six-membered heterocycloalkyl group;
R⁶ is alkyl, aryl, alkylaryl, halo, —OH, —O—(C₁-C₆ alkyl), haloalkyl, —OCF₃, —NR⁴R⁵, phenyl, —NO₂, —CO₂R⁴, —CON(R⁴)₂ or —CN;
R¹² is alkyl, —OH, —O-alkyl, or —F;
R¹³ is alkyl, —OH, —O-alkyl, or —F;
each occurrence of R²⁰ is independently —H or C₁-C₆ alkyl;
R³⁰ is heterocycloalkyl;
each occurrence of R⁴⁵ is independently H, alkyl, alkylaryl, or aryl, wherein an aryl group or the aryl moiety of an alkylaryl group can be optionally substituted with from 1 to 3 substituents which are the same or different, and are independently selected from haloalkyl, —OH, halo, alkyl, —NO₂, and —CN;
M¹ and M² are each independently CH, CF or N;
Y is —CH₂—, —C(O)—, —C(NOR²⁰)— or —C(S)—;
Z is alkylene;
a is 0, 1 or 2;
b is 0, 1 or 2;
c is 0, 1 or 2;
e is an integer ranging from 0 to 5;
m is 1 or 2;
n is 1, 2 or 3, such that when M¹ is nitrogen, n is 2 or 3; and
p is 1, 2 or 3, such that when M² is nitrogen, p is 2 or 3,
wherein the condition is diabetes, a diabetic complication, impaired glucose tolerance or impaired fasting glucose.

2. The method of claim 1, wherein the condition is diabetes.

3. The method of claim 2, wherein for the compound of formula (I), R¹ is aryl or heteroaryl, or R¹ is taken together with X to form: 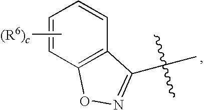
wherein an aryl or heteroaryl group can be optionally substituted with halo, alkyl or substituted alkyl.

4. The method of claim 3, wherein for the compound of formula (I), R¹ is phenyl, 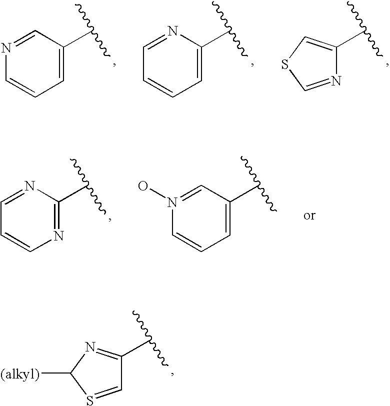
or R¹ is taken together with X to form: 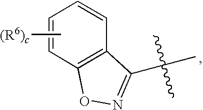
wherein c is 0 or 1, such that when c is 1 then R⁶ is —F, and wherein a phenyl group may be optionally and independently substituted with one or more of —Cl, —F or trifluoromethyl.

5. The method of claim 6, wherein R¹ is

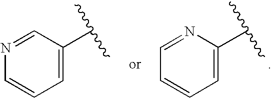

6. The method of claim 2, wherein for the compound of formula (I), X is —C(NOR³)—, and R³ is H or alkyl.

7. The method of claim 6, wherein R³ is H, methyl or ethyl.

8. The method of claim 7, wherein R³ is methyl.

9. The method of claim 2, wherein for the compound of formula (I), M¹ and M² are each CH.

10. The method of claim 2, wherein for the compound of formula (I), n is 2; a is 0 or 1; b is 0 or 1; c is 0 or 1, such that when c is 1 then R⁶ is halo; e is an integer ranging from 1 to 5; and p is 2.

11. The method of claim 2, wherein for the compound of formula (I), Y is —C(O)—.

12. The method claim 2, wherein for the compound of formula (I), Z is

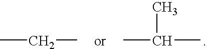

13. The method of claim 2, wherein for the compound of formula (I), R² is a six membered heteroaryl ring.

14. The method of claim 13 wherein R² is pyridyl or pyrimidinyl.

15. The method of claim 2 wherein R² is

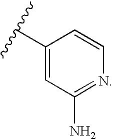

16. The method of claim 2 wherein R² is

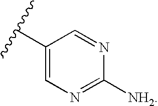

17. The method of claim 2, wherein for the compound of formula (I), R⁴ is H or lower alkyl; R⁵ is H, C₁-C₆alkyl, or —C(O)R⁴; R¹² is H, alkyl, —OH or —F; and R¹³ is H, alkyl, —OH or —F.

18. The method of claim 2, wherein the compound of formula (I) is a compound of formula (Ia): 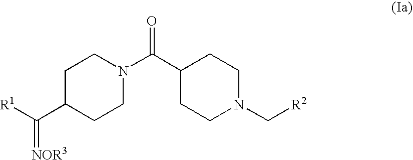
or a pharmaceutically acceptable salt, solvate, ester or prodrug thereof, wherein R¹, R² and R³ are defined in claim 1.

19. The method of claim 18, wherein R¹ is aryl or heteroaryl.

20. The method of claim 19, wherein R¹ is phenyl, pyridyl or

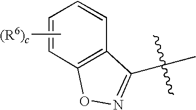

21. The method of claim 20, wherein R¹ is pyridyl.

22. The method of claim 18, wherein R² is a 6-membered heteroaryl.

23. The method of claim 22, wherein R² is pyridyl or pyrimidinyl.

24. The method of claim 18, wherein R² is:

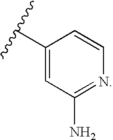

25. The method of claim 18, wherein R² is:

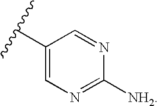

26. The method of claim 20, wherein R³ is alkyl.

27. The method of claim 26, wherein R³ is methyl.

28. The method of claim 19, wherein R² is six-membered heteroaryl and R³ is alkyl.

29. The method of claim 2, wherein the one or more compounds of formula (I) are selected from: 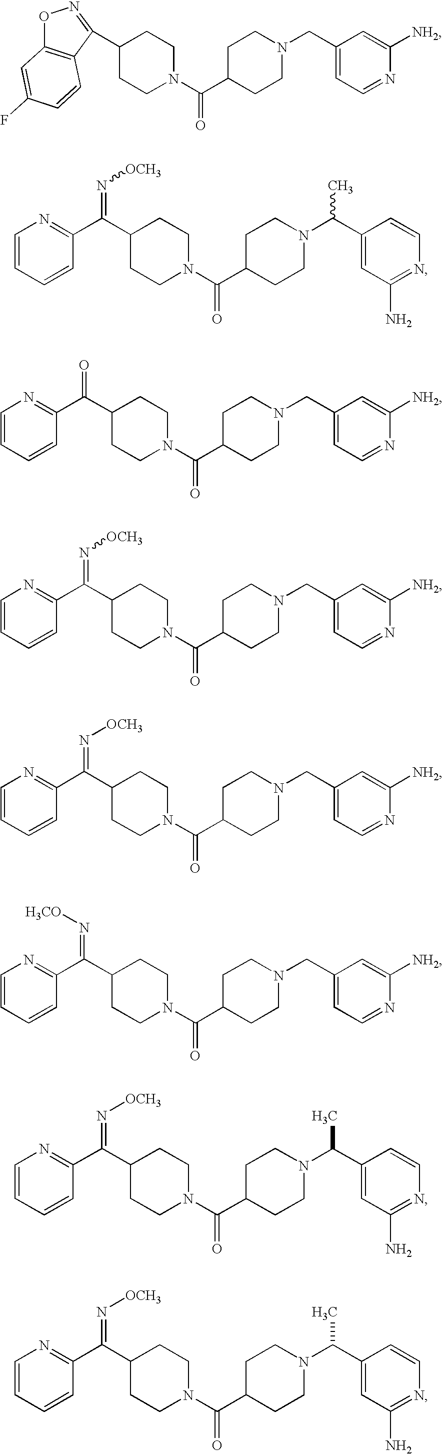 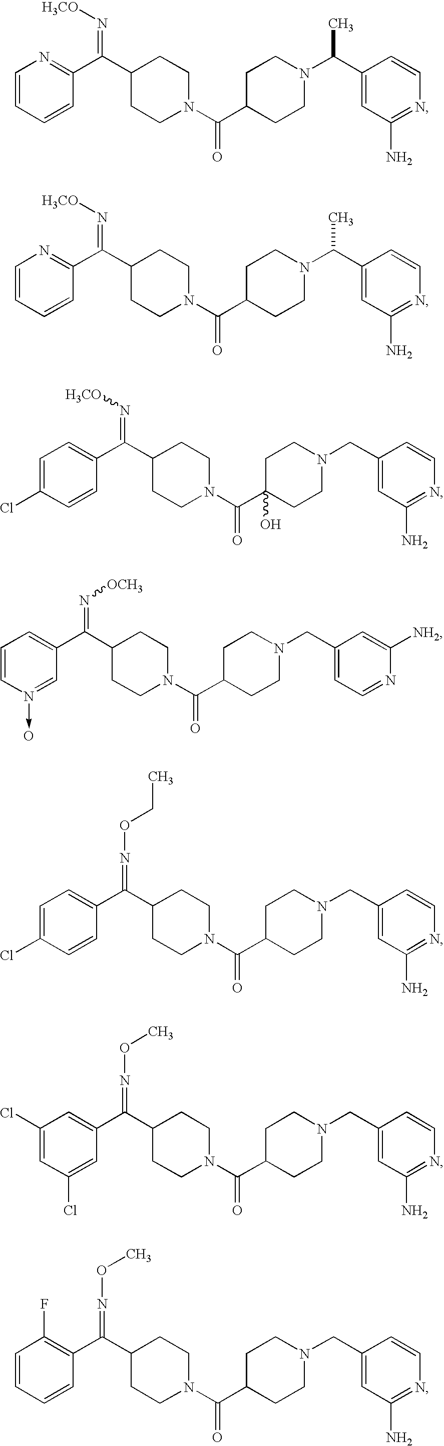 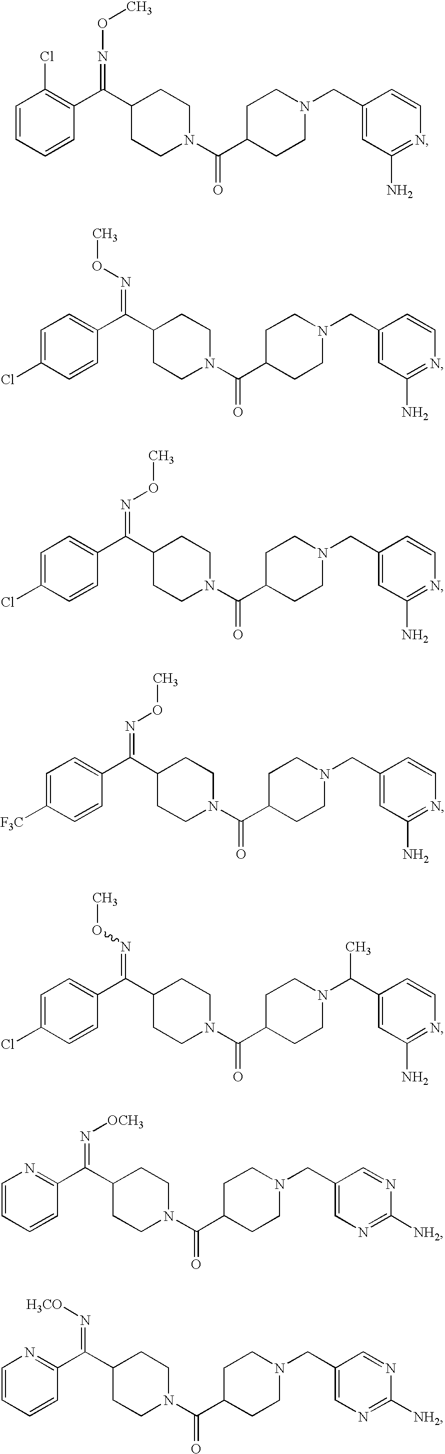 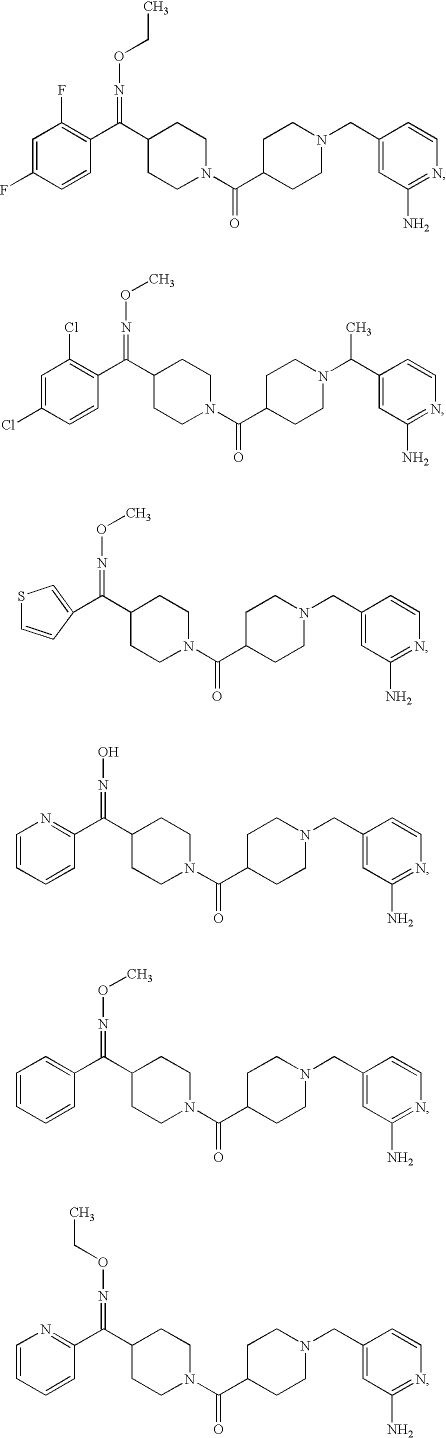 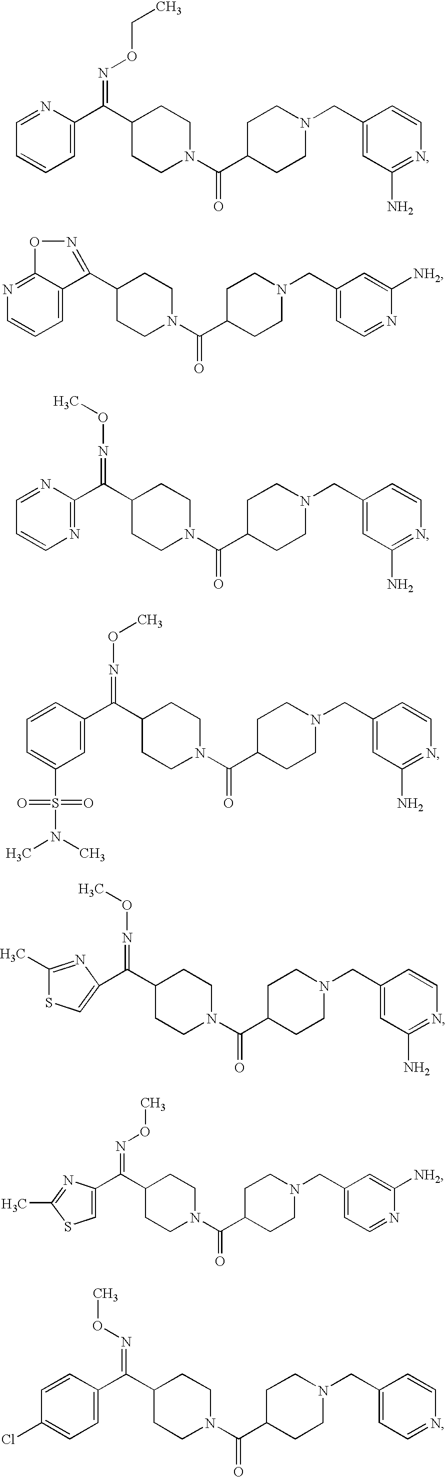 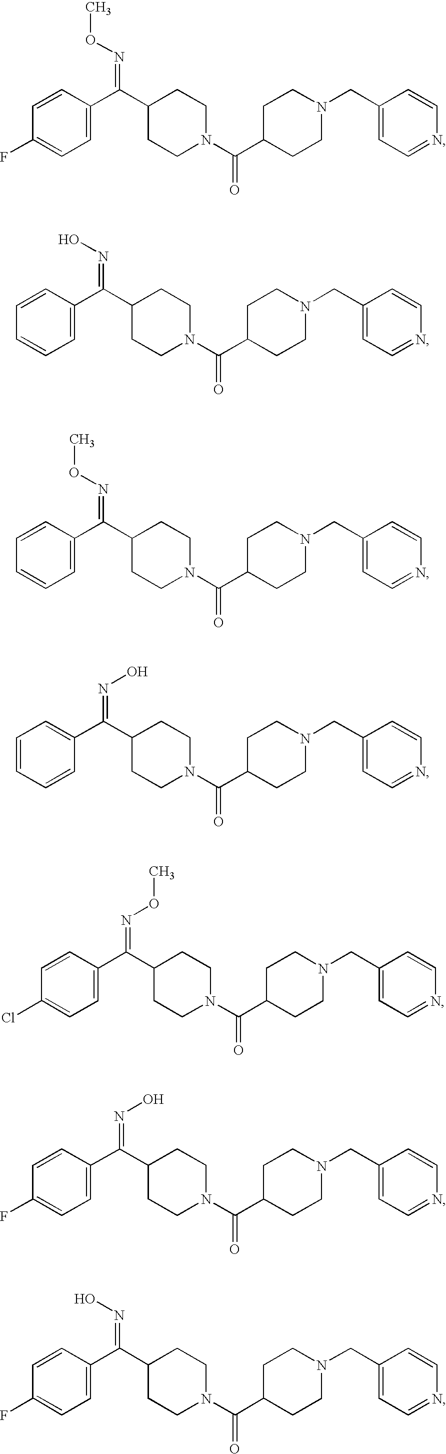 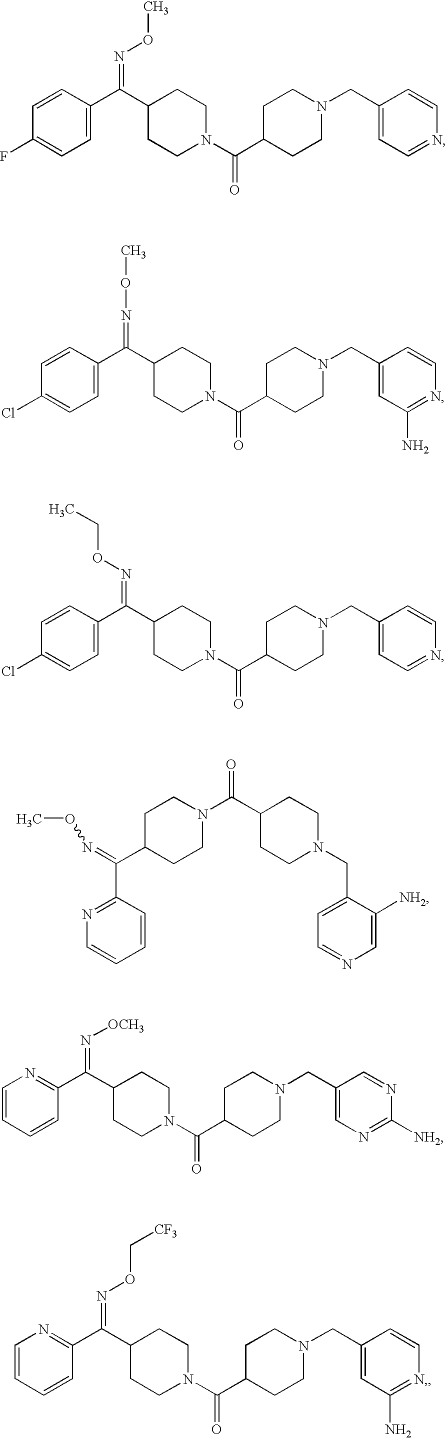 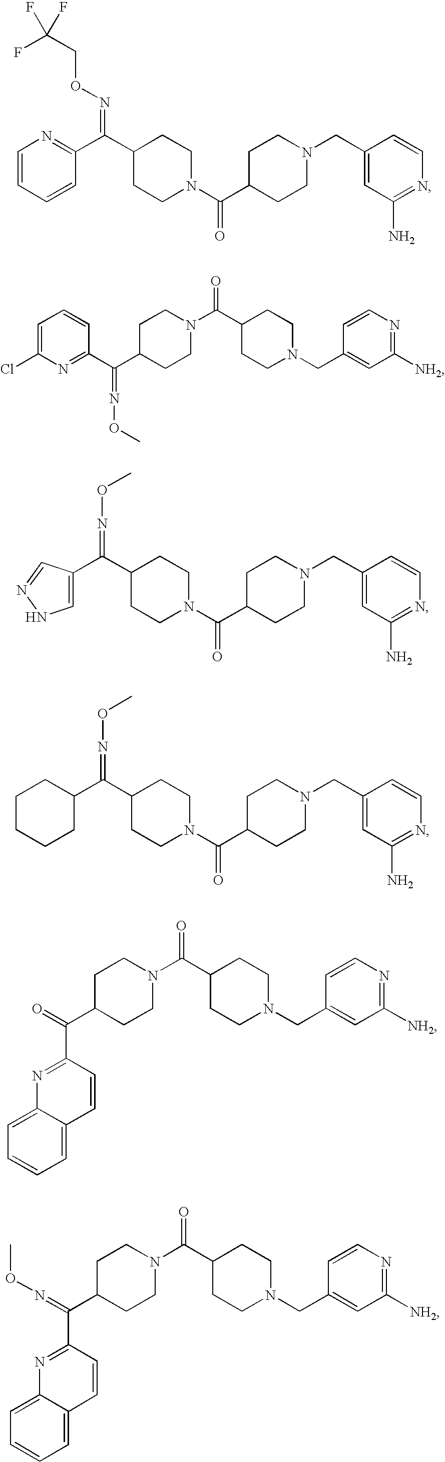 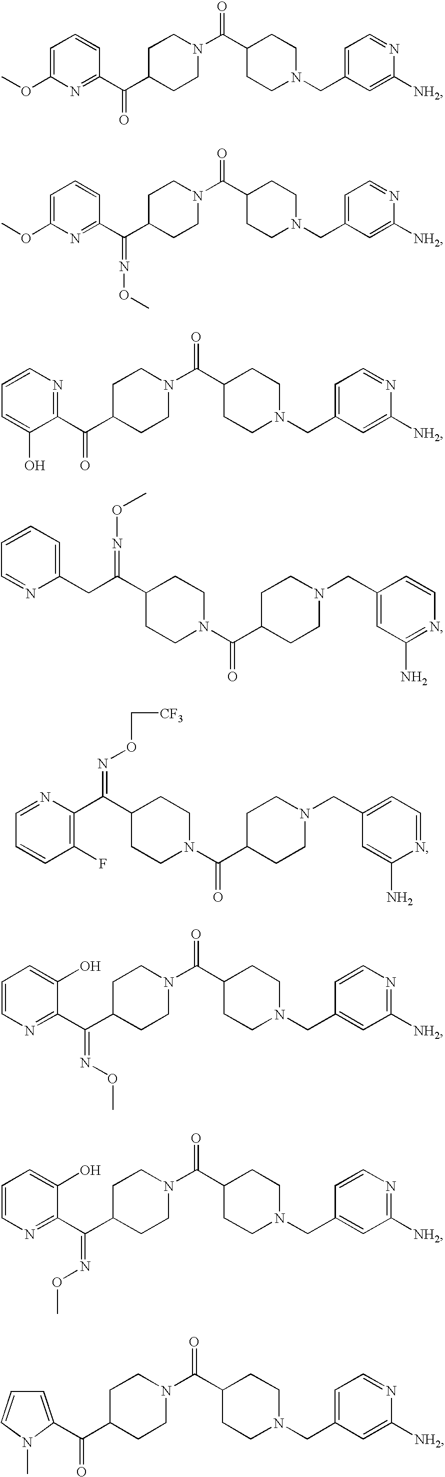 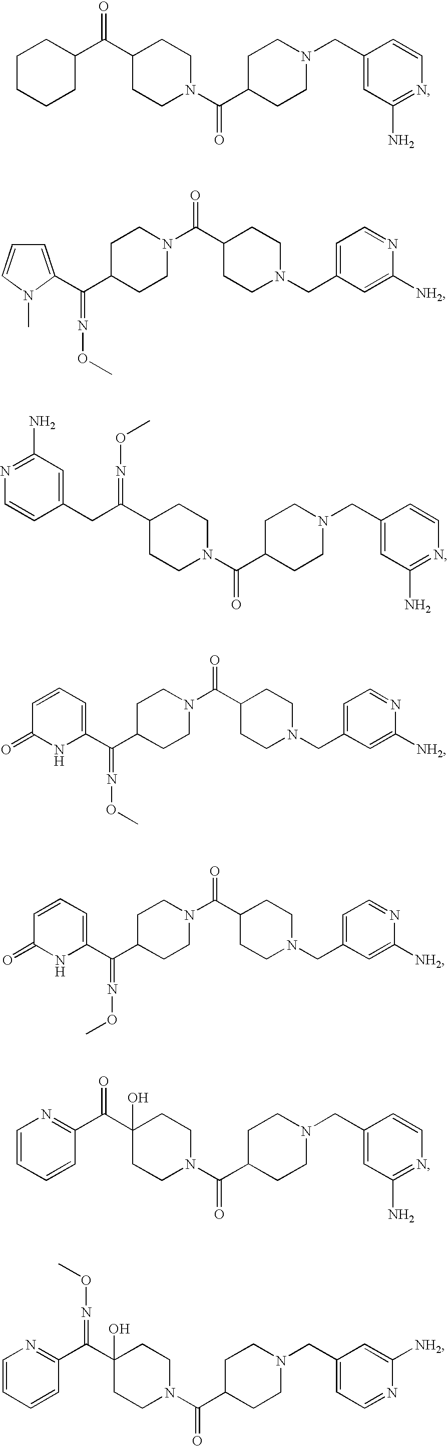 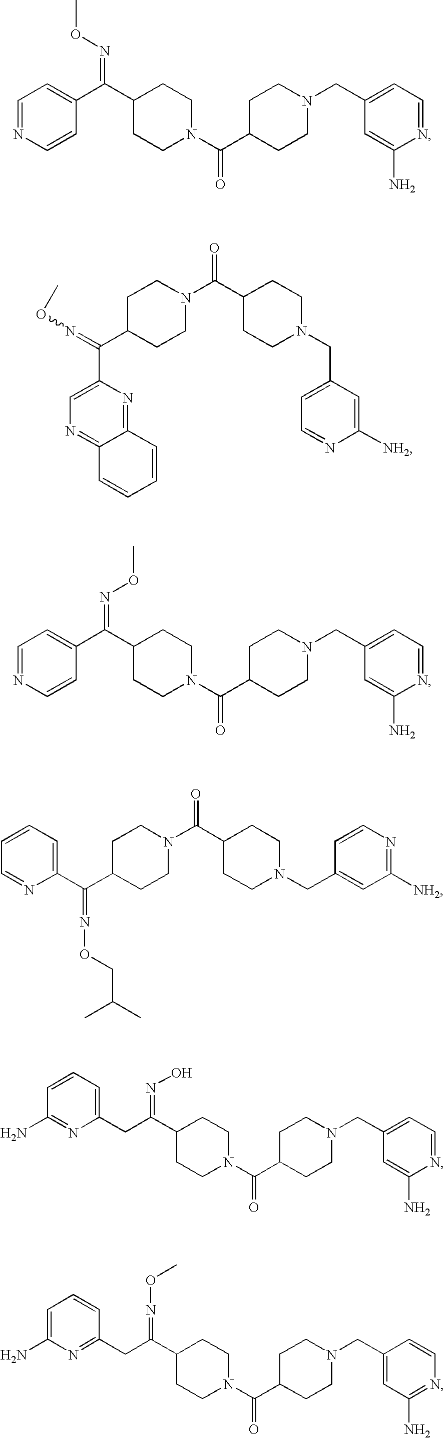 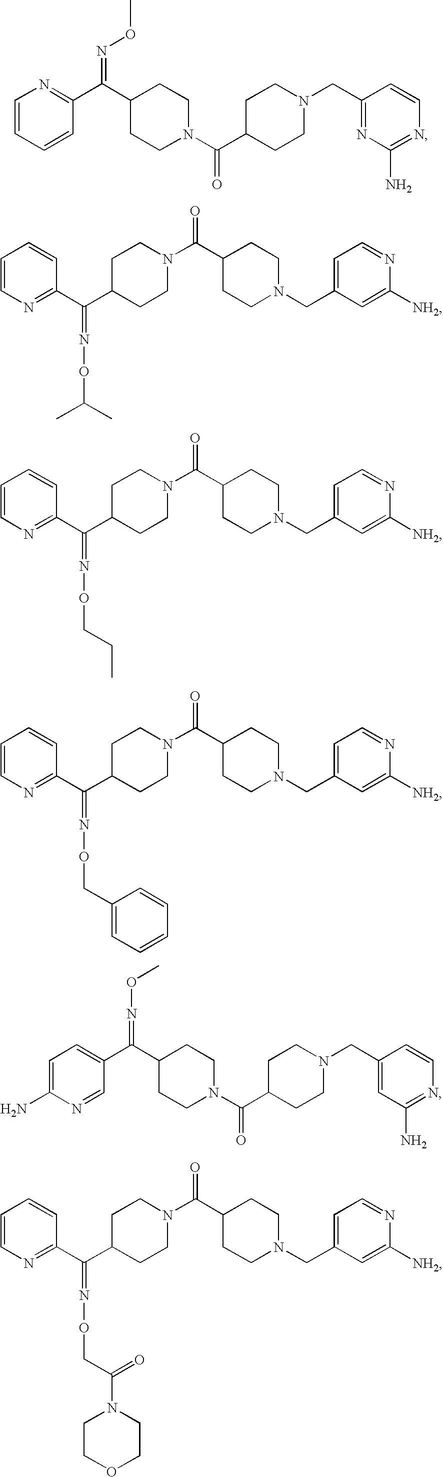 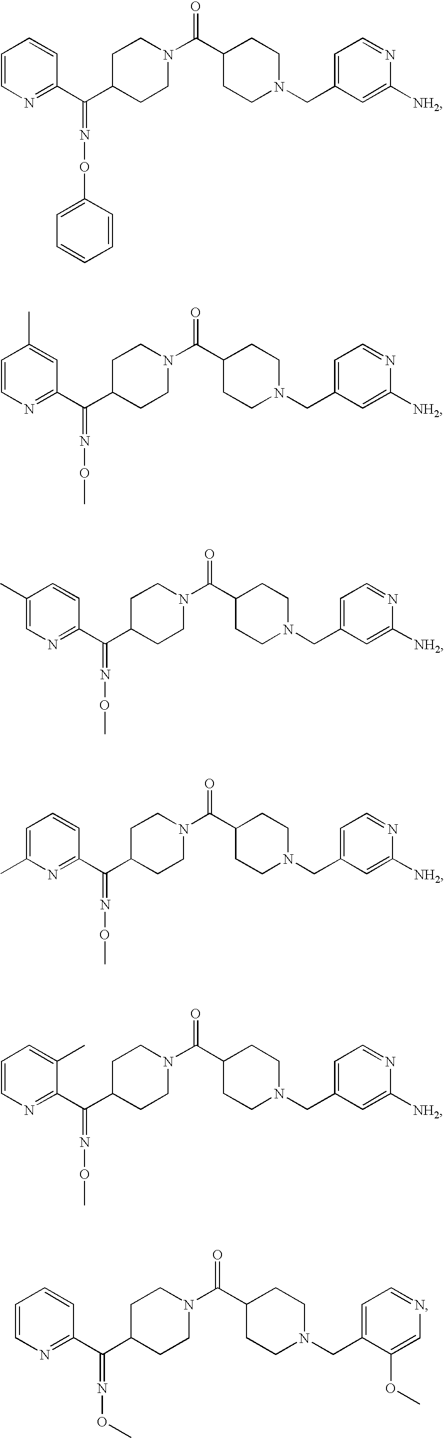 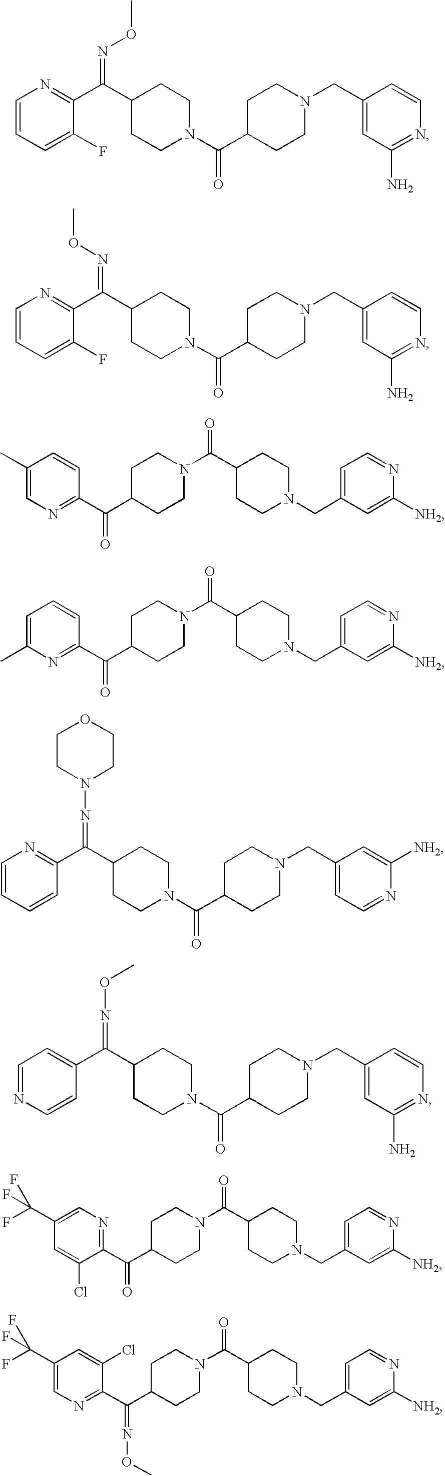 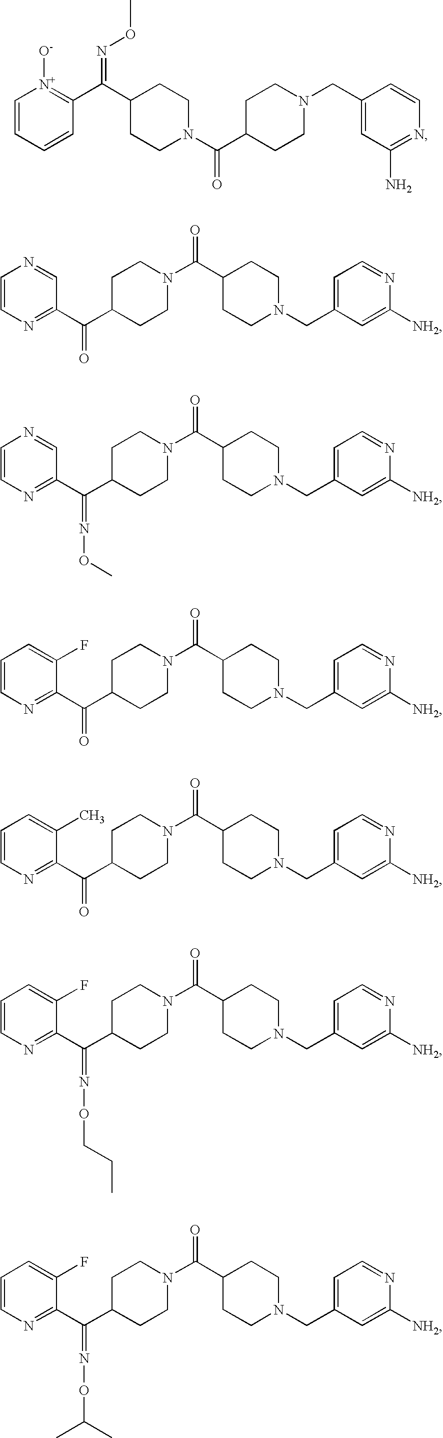 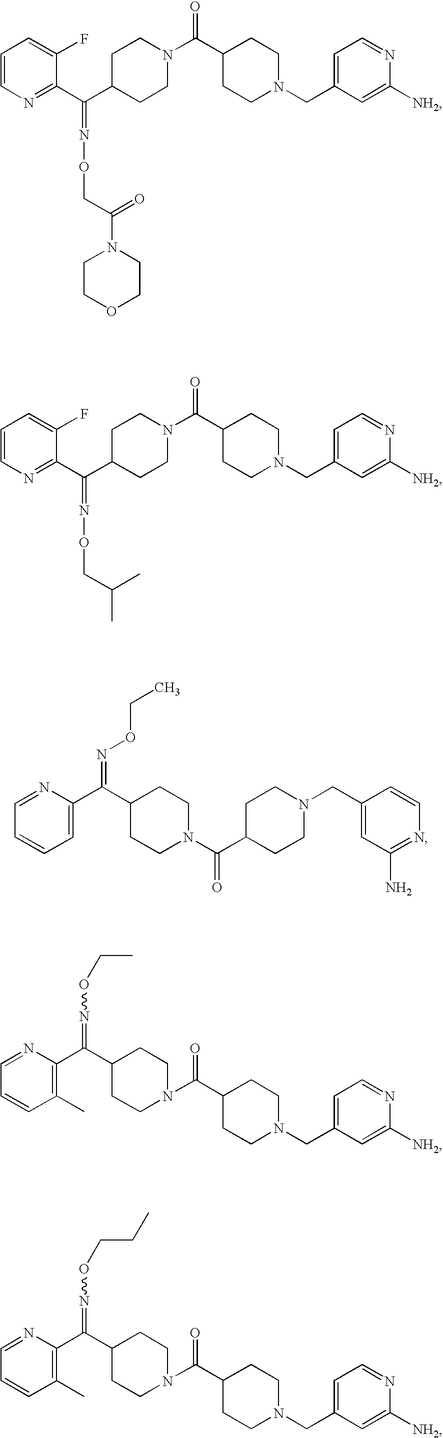 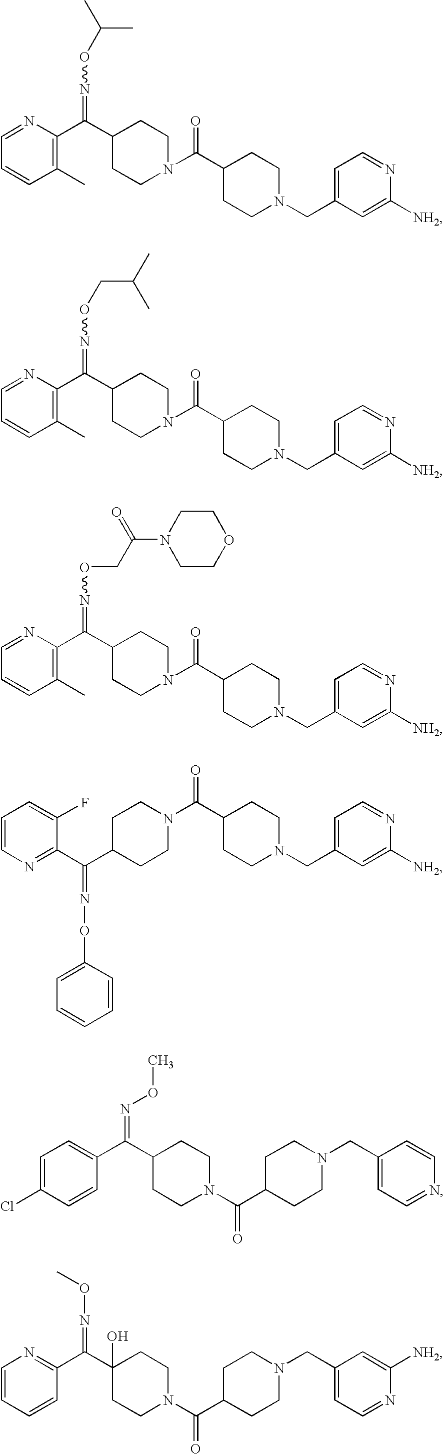 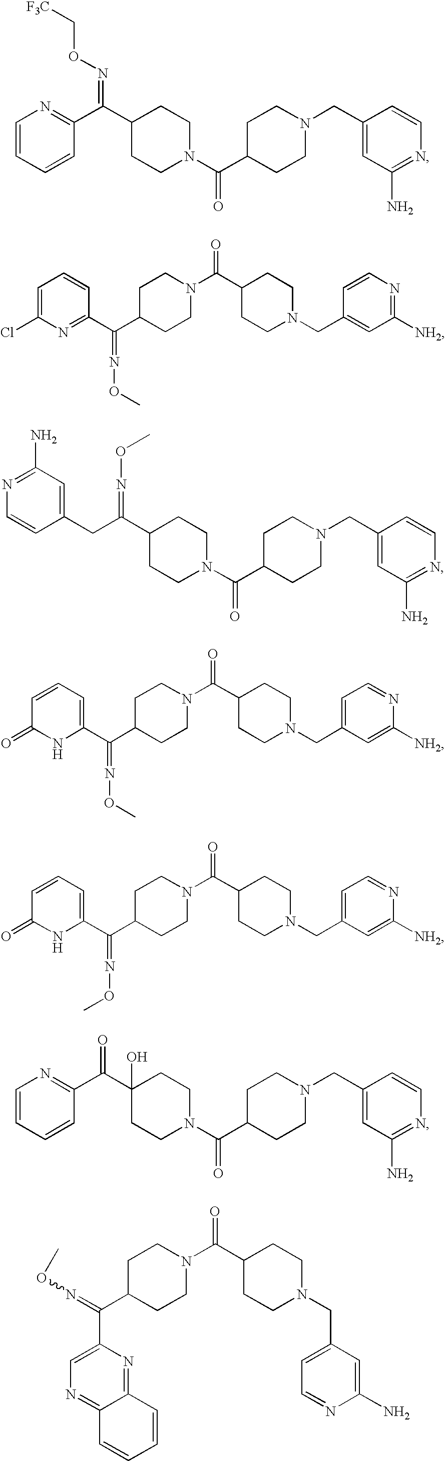 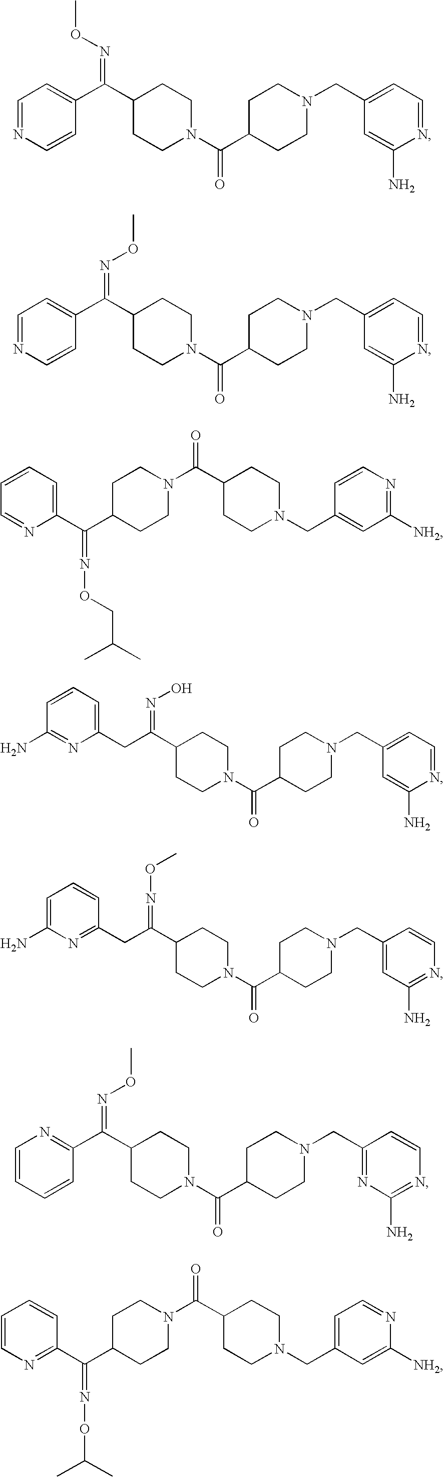 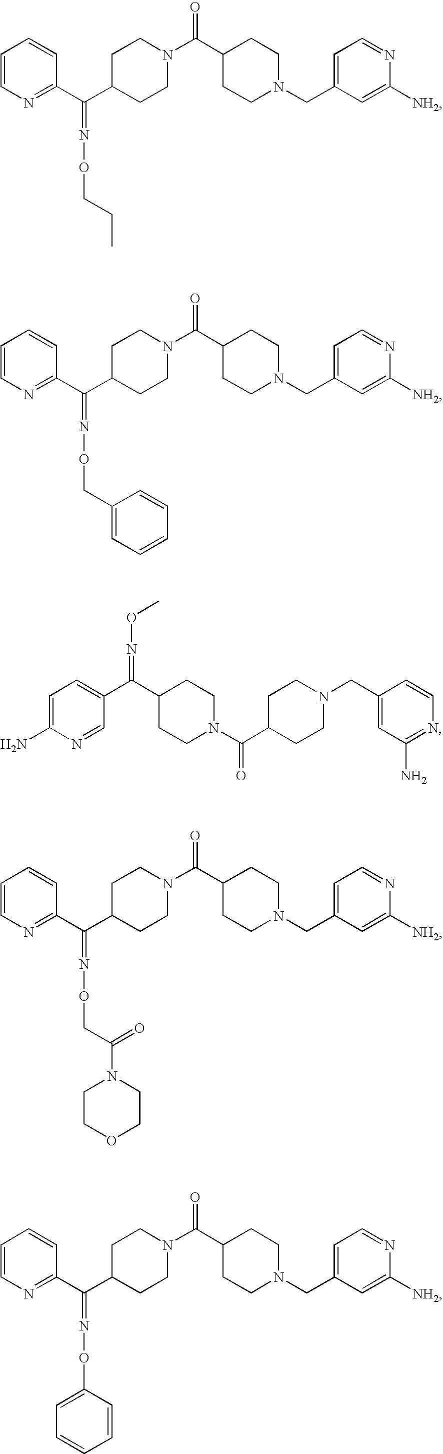 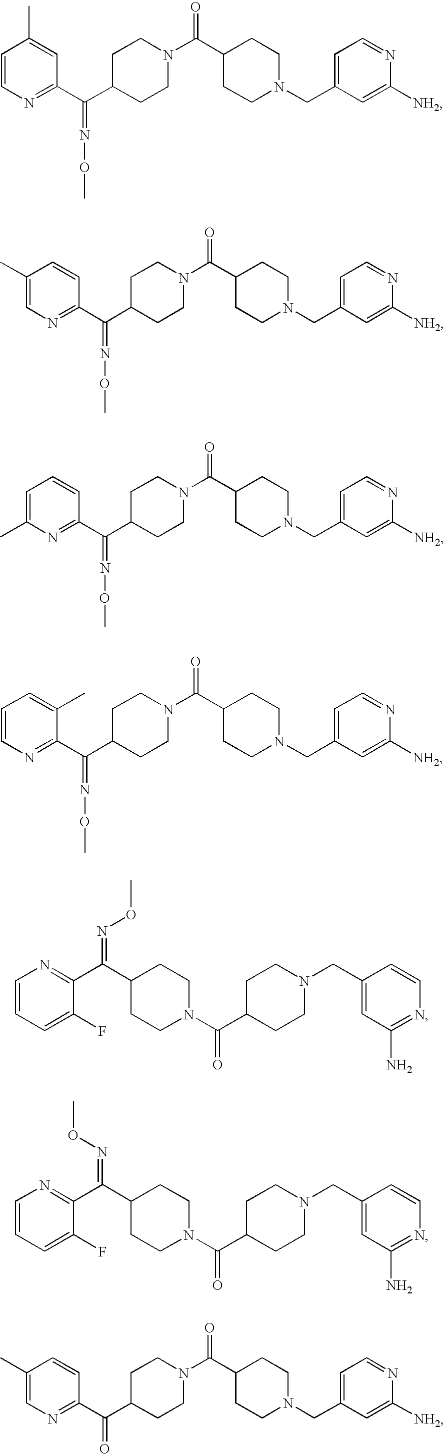 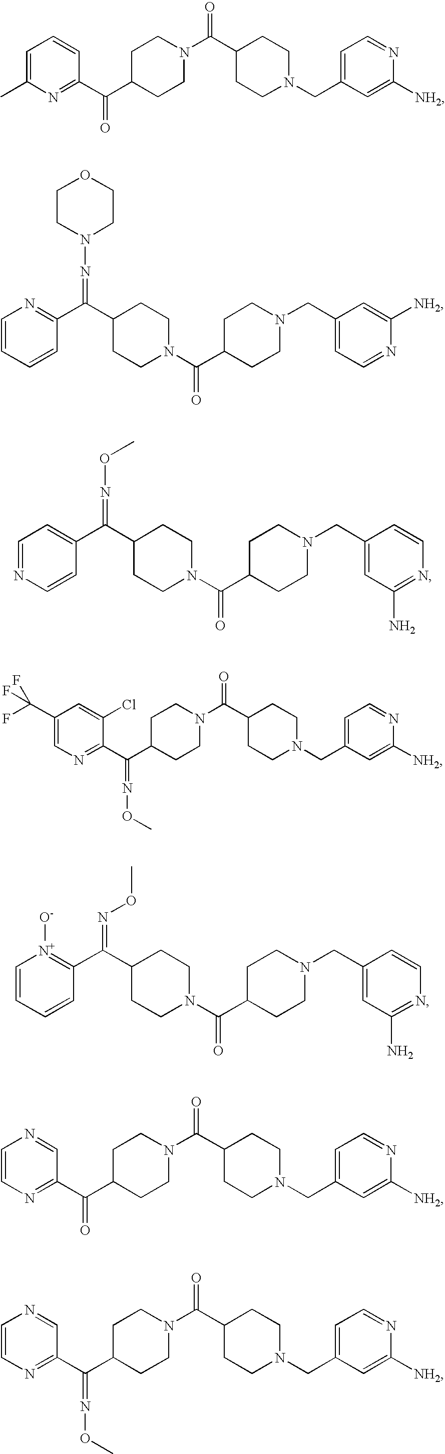 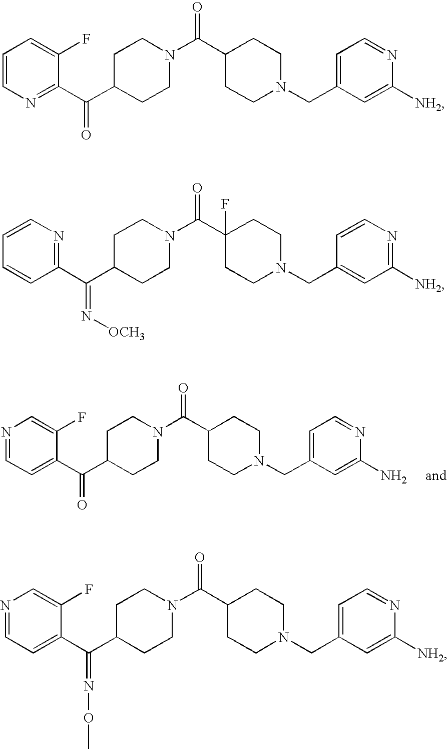
or a pharmaceutically acceptable salt, solvate, ester or prodrug thereof.

30. The method of claim 29, wherein the compound of formula (I) is 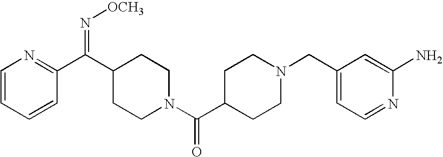
or a pharmaceutically acceptable salt, solvate, ester or prodrug thereof.

31. The method of claim 29, wherein the compound of formula (I) is 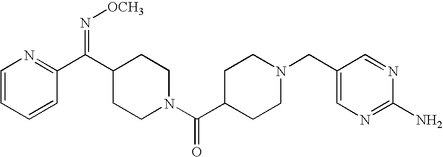
or a pharmaceutically acceptable salt, solvate, ester or prodrug thereof.

32. The method of claim 2, further comprising administering to the patient an additional antidiabetic agent that is not a compound of formula (I), wherein the amounts of the compound of Formula (I) and the additional antidiabetic agent are together effective to treat diabetes.

33. The method of claim 32, wherein the additional antidiabetic agent is selected from a sulfonylurea, an insulin sensitizer, an α-glucosidase inhibitor, an insulin secretagogue, an antiobesity agent, a meglitinide, insulin or an insulin-containing composition.

34. The method of claim 33, wherein the additional antidiabetic agent is an insulin sensitizer or a sulfonylurea.

35. The method of claim 34, wherein the insulin sensitizer is a PPAR activator or a DPP-IV inhibitor.

36. The method of claim 33, wherein the additional antidiabetic agent is an antiobesity agent.

37. The method of claim 36, wherein the antiobesity agent is selected from: a neuropeptide Y antagonist, an MCR4 agonist, an MCH receptor antagonist, a protein hormone, an AMP kinase activator, and a lipase inhibitor.

38. The method of claim 36, wherein antiobesity agent is orlistat, leptin, or adiponectin.

39. The method of claim 2, wherein the diabetes is type I diabetes.

40. The method of claim 2, wherein the diabetes is type II diabetes.

41. The method of claim 1, wherein the condition treated is a diabetic complication.

42. The method of claim 41, wherein the diabetic complication is diabetic cataract, glaucoma, retinopathy, neuropathy, nephropathy, gangrene of the feet, immune-complex vasculitis, systemic lupsus erythematosus, atherosclerotic coronary arterial disease, peripheral arterial disease, nonketotic hyperglycemic-hyperosmolar coma, foot ulcers or joint problems.

43. The method of claim 42, wherein the diabetic complication is neuropathy, retinopathy or nephropathy.

44. The method of claim 1, wherein the condition treated is impaired glucose tolerance.

45. The method of claim 1, wherein the condition treated is impaired fasting glucose.

46. A method for treating pain in a patient, comprising administering to the patient an effective amount of one or more compounds having the formula: 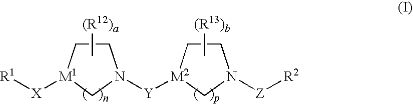
or a pharmaceutically acceptable salt, solvate, ester or prodrug thereof, wherein:
R¹ is aryl, heteroaryl, heterocycloalkyl, alkyl, cycloalkyl or alkylaryl, each of which can be optionally substituted with from 1 to 4 substituents, which are the same or different, and are independently selected from halo, —OH, —O-alkyl, haloalkyl, —OCF₃, —NR⁴R⁵, phenyl, —NO₂, —CO₂R⁴, —CON(R⁴)₂, —S(O)ₘN(R²⁰)₂ and —CN, or R¹ and X are taken together to form: 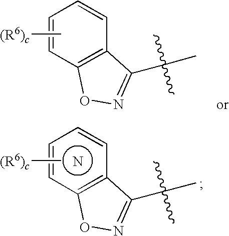
X is —C(O)—, —C(NOR³)—, —C(NNR⁴R⁵)—, 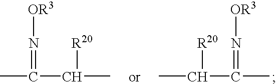
R² is a five or six-membered heteroaryl group, wherein a six-membered heteroaryl group contains 1 or 2 nitrogen ring atoms with the remaining ring atoms being carbon, and a five-membered heteroaryl group contains 1 or 2 hetero ring atoms selected from nitrogen, oxygen, and sulfur, with the remaining ring atoms being carbon; and wherein a five or six membered heteroaryl group can be optionally substituted with from 1 to 3 substituents, which are the same or different, and are independently selected from halo, —OH, alkyl, —O-alkyl, haloalkyl, —OCF₃, —NR⁴R⁵, phenyl, —NO₂, —CO₂R⁴, —CON(R⁴)₂, —CH₂NR⁴R⁵, —(N)C(NR⁴R⁵)₂, and —CN;
R³ is hydrogen, alkyl, aryl, heteroaryl, heterocycloalkyl, arylalkyl, haloalkyl, —CH₂CF₃, —(CH₂)ₑ—C(O)N(R⁴)₂, —(CH₂)ₑ—C(O)OR⁴ or —(CH₂)ₑ—C(O)R³⁰, wherein an aryl, heteroaryl or heterocycloalkyl group, or the aryl portion of an arylalkyl group can be optionally substituted with from 1 to 3 substituents, which are the same or different, and are independently selected from halo, —OH, —OCF₃, haloalkyl, —CN, —N(R⁴⁵)₂, —CO₂R⁴⁵ and —C(O)N(R⁴⁵)₂;
each occurrence of R⁴ is independently hydrogen, alkyl, aryl or alkylaryl, wherein an aryl group or the aryl moiety of an alkylaryl group can be optionally substituted with 1 to 3 substituents, which are the same or different, and are independently selected from halo, haloalkyl, —OCF₃, —OH, —N(R⁴⁵)₂, —CO₂R⁴⁵, —C(O)N(R⁴⁵)₂ and —CN;
R⁵ is hydrogen, alkyl, —C(O)R⁴, —C(O)₂R⁴ or —C(O)N(R⁴)₂, or R⁴ and R⁵ taken together with the nitrogen atom to which they are both attached, join to form a five- or six-membered heterocycloalkyl group;
R⁶ is alkyl, aryl, alkylaryl, halo, —OH, —O—(C₁-C₆ alkyl), haloalkyl, —OCF₃, —NR⁴R⁵, phenyl, —NO₂, —CO₂R⁴, —CON(R⁴)₂ or —CN;
R¹² is alkyl, —OH, —O-alkyl, or —F;
R¹³ is alkyl, —OH, —O-alkyl, or —F;
each occurrence of R²⁰ is independently —H or C₁-C₆ alkyl;
R³⁰ is heterocycloalkyl;
each occurrence of R⁴⁵ is independently H, alkyl, alkylaryl, or aryl, wherein an aryl group or the aryl moiety of an alkylaryl group can be optionally substituted with from 1 to 3 substituents which are the same or different, and are independently selected from haloalkyl, —OH, halo, alkyl, —NO₂, and —CN;
M¹ and M² are each independently CH, CF or N;
Y is —CH₂—, —C(O)—, —C(NOR²⁰)— or —C(S)—;
Z is alkylene;
a is 0, 1 or 2;
b is 0, 1 or 2;
c is 0, 1 or 2;
e is an integer ranging from 0 to 5;
m is 1 or 2;
n is 1, 2 or 3, such that when M¹ is nitrogen, n is 2 or 3; and
p is 1, 2 or 3, such that when M² is nitrogen, p is 2 or 3.

47. The method of claim 46, wherein the compound of formula (I) is a compound of claim 29 or a pharmaceutically acceptable salt, solvate, ester or prodrug thereof.

48. The method of claim 47, wherein the compound of formula (I) is 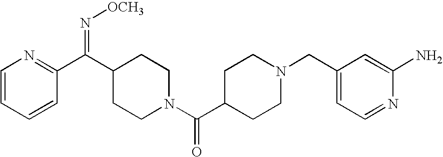
or a pharmaceutically acceptable salt, solvate, ester or prodrug thereof.

49. The method of claim 47, wherein the compound of formula (I) is 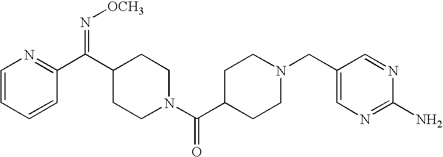
or a pharmaceutically acceptable salt, solvate, ester or prodrug thereof.

50. The method of claim 46, further comprising administering to the patient an additional analgesic agent that is not a compound of formula (I), wherein the amounts of the one or more compounds of Formula (I) and the additional analgesic agent are together effective to treat diabetes.

51. The method of claim 50, wherein the additional analgesic agent is acetaminophen, an NSAID, an opiate or a tricyclic antidepressant.

52. The method of claim 51, wherein the NSAID is aspirin, ibuprofen, naproxen, celecoxib, etoricoxib, lumiracoxib or parecoxib.

53. The method of claim 51, wherein the opiate is an anilidopiperidine, a phenylpiperidine, a diphenylpropylamine derivative, a benzomorphane derivative, an oripavine derivative or a morphinane derivative.

54. The method of claim 53, wherein the opiate or is morphine, codeine, oxycodone, hydrocodone, diamorphine, pethidine, vicodin, percocet, percodan, norco, dilaudid, darvocet, lorcet, pentazocine, tramadol or fentanyl.

55. A composition comprising a compound of claim 1, an additional antidiabetic agent that is not a compound of formula (I), and a pharmaceutically acceptable carrier.

56. The composition of claim 55, wherein the additional antidiabetic agent is selected from a sulfonylurea, an insulin sensitizer, an α-glucosidase inhibitor, an insulin secretagogue, an anti-obesity agent, a meglitinide, insulin or an insulin-containing composition.

57. The composition of claim 56, wherein the additional antidiabetic agent is an insulin sensitizer or a sulfonylurea.

58. The composition of claim 57, wherein the insulin sensitizer is a PPAR activator or a DPP-IV inhibitor.

59. The composition of claim 55, wherein the additional antidiabetic agent is an antiobesity agent.

60. The composition of claim 59, wherein the antiobesity agent is selected from: a neuropeptide Y antagonist, an MCR4 agonist, an MCH receptor antagonist, a protein hormone, an AMP kinase activator, and a lipase inhibitor.

61. The composition of claim 60, wherein antiobesity agent is orlistat, leptin, or adiponectin.

62. A composition comprising a compound of claim 1, an additional analgesic agent that is not a compound of formula (I), and a pharmaceutically acceptable carrier.

63. The composition of claim 62, wherein the additional analgesic agent is acetaminophen, an NSAID, an opiate or a tricyclic antidepressant.

64. The composition of claim 63, wherein the NSAID is a salicylate, an arylalkanoic acid, a profen, a fenamic acid, a pyrazolidine derivative, a coxib, an oxicam or a sulfonanilide.

65. The composition of claim 64, wherein the NSAID is aspirin, ibuprofen, naproxen, celecoxib, etoricoxib, lumiracoxib or parecoxib.

66. The composition of claim 63, wherein the additional analgesic agent is an opiate.

67. The composition of claim 66, wherein the opiate is an anilidopiperidine, a phenylpiperidine, a diphenylpropylamine derivative, a benzomorphane derivative, an oripavine derivative or a morphinane derivative.

68. The composition of claim 67, wherein the opiate is morphine, codeine, oxycodone, hydrocodone, diamorphine, pethidine, vicodin, percocet, percodan, norco, dilaudid, darvocet, lorcet, pentazocine, tramadol or fentanyl.
